SEQUENCE LISTING (3 sequences)

SEQ ID NO: 1 - protein, 4 aa, Mus sp.
  MOD_RES (2)..(2): D-phenylalanine
HFRW

SEQ ID NO: 2 - protein, 4 aa, Mus sp.
  MOD_RES (2)..(2): D-2-naphthyl-alanine
HARW

SEQ ID NO: 3 - protein, 12 aa, Artificial Sequence
  Description of Artificial Sequence Synthetic peptide
PGPGPGPGPGPG

(19) United States
(12) Patent Application Publication
(10) Pub. No.: US 2018/0360972 A1
(43) Pub. Date: Dec. 20, 2018

(54) THERAPEUTIC COMPOUNDS

(71) Applicant: REGENTS OF THE UNIVERSITY OF MINNESOTA, Minneapolis, MN (US)

(72) Inventors: Carrie Haskell-Luevano, Minneapolis, MN (US); Cody James Lensing, Minneapolis, MN (US)

(21) Appl. No.: 15/969,670

(22) Filed: May 2, 2018

Related U.S. Application Data
(60) Provisional application No. 62/500,445, filed on May 2, 2017.

(51) Int. Cl. A61K 47/54; A61P 3/04; A61K 38/07; C07K 5/11; A23L 33/18

(52) U.S. Cl. CPC A61K 47/54; A61P 3/04; A61K 38/07; A23V 2002/00; A23L 33/18; C07K 2319/00; C07K 5/1019

(57) ABSTRACT

The invention provides compounds having the general formula I:

Y—X—Z I

and salts thereof, wherein the variables X, Y, and Z have the meaning as described herein, and compositions containing such compounds and methods for using such compounds and compositions.

### RELATED APPLICATION

This application claims the benefit of priority of U.S. Provisional Application Ser. No. 62/500,445 filed on May 2, 2017, which application is incorporated by reference herein.

### GOVERMENT FUNDING

This invention was made with government support under R01 DK091906 and R01 DK108893 awarded by the National Institutes of Health. The government has certain rights in the invention.

### BACKGROUND OF THE INVENTION

G protein-coupled receptors (GPCRs) are highly sought after drug targets in the pharmaceutical industry with approximately 30-40% of drugs targeting them (Rask-Andersen, M. et al. Nature Reviews Drug Discovery 2011, 10, 579-590 and Santos, R. et al. Nature Reviews Drug Discovery 2017, 16, 19-34). Classically, medicinal chemists targeted GPCRs as monomeric units; however increasing evidence has shown GPCRs form dimers with themselves (homodimers) and with other GPCRs (heterodimers) (Ferre, S. et al. Pharmacol. Rev. 2014, 66, 413-434 and Ferre, S. et al. Trends Pharmacol. Sci. 2015, 36, 145-152). Targeting GPCR homodimers' and heterodimers' distinct and exploitable functions may yield a revolution in GPCR targeting therapeutics. Although ligands targeting heterodimers have shown much promise in both in vitro and in vivo preclinical studies (Daniels, D. J. et al. Proc. Natl. Acad. Sci. U. S. A. 2005, 102, 19208-19213; Smeester, B. A. et al. Eur. I Pharmacol. 2014, 743, 48-52; Le Naour, M. et al. J. Med. Chem. 2013, 56, 5505-5513; Akgün, E. et al. J. Med. Chem. 2015, 58, 8647-8657 and Portoghese, P. S. et. al. ACS Chem. Neurosci. 2017, 8, 426-428) there has been limited development of ligands targeting the allosterism that can occur within homodimers.

Pharmacologically targeting homodimers possess a unique conundrum: How to target and detect a homodimer when the two receptors comprising it are structurally similar, usually respond to the same ligands, and appear to have the same propensity to signal in standard cell culture assays? Various groups have devised clever strategies around these problems to demonstrate the functional consequences of asymmetric homodimers (Han, Y. et al. Nat. Chem. Biol. 2009, 5, 688-695; Pellissier, L. P. et al. J. Biol. Chem. 2011, 286, 9985-9997; Teitler, M. et al. Pharmacol. Ther. 2012, 133, 205-217; Comps-Agrar, L. et al. EMBO J. 2011, 30, 2336-2349; Pin, J. P. et al. Febs J. 1 2005, 272, 2947-2955; Hlavackova, V. et al. EMBO J. 2005, 24, 499-509; Prezeau, L. et al. Neuropharmacology 2005, 49, 267-267; Kniazeff, J. et al. Nat. Struct. Mol. Biol. 2004, 11, 706-713; Kniazeff, J. et al. J. Neurosci. 2004, 24, 370-377; Zylbergold, P. et al. Nat. Chem. Biol. 2009, 5, 608-609; Szalai, B. et al. Biochem. Pharmacol. 2012, 84, 477-485; Damian, M. et al. EMBO J. 2006, 25, 5693-5702; Brock, C. et al. J. Biol. Chem. 2007, 282, 33000-33008; Sartania, N et al. Cell. Signal. 2007, 19, 1928-1938; Gracia, E. et al. Neuropharmacology 2013, 71, 56-69; Chapman, K. L. et al. Biochim. Biophys. Acta. 2013, 1828, 535-542; Orcel, H. et al. Mol. Pharmacol. 2009, 75, 637-647 and Iglesias, A. et al. Eur. J. Pharmacol. 2017, 800, 63-69). Some of these groups focus on demonstrating subtle changes in pharmacology utilizing strategically designed in vitro experiments and other groups exploited receptor mutation strategies in order to differentiate between the two protomers making up the dimer (Han, Y. et al. Nat. Chem. Biol. 2009, 5, 688-695; Pellissier, L. P. et al. J. Biol. Chem. 2011, 286, 9985-9997; Teitler, M. et al. Pharmacol. Ther. 2012, 133, 205-217; Comps-Agrar, L. et al. EMBO J. 2011, 30, 2336-2349; Pin, J. P. et al. Febs J. 2005, 272, 2947-2955; Hlavackova, V. et al. EMBO J. 2005, 24, 499-509; Prezeau, L. et al. Neuropharmacology 2005, 49, 267-267; Kniazeff, J. et al. Nat. Struct. Mol. Biol. 2004, 11, 706-713; Kniazeff, J. et al. J. Neurosci. 2004, 24, 370-377; Zylbergold, P. et al. Nat. Chem. Biol. 2009, 5, 608-609; Szalai, B. et al. Biochem. Pharmacol. 2012, 84, 477-485; Damian, M. et al. EMBO J. 2006, 25, 5693-5702; Brock, C. et al. J. Biol. Chem. 2007, 282, 33000-33008; Sartania, N et al. Cell. Signal. 2007, 19, 1928-1938; Gracia, E. et al. Neuropharmacology 2013, 71, 56-69; Chapman, K. L. et al. Biochim. Biophys. Acta. 2013, 1828, 535-542 and Orcel, H. et al. Mol. Pharmacol. 2009, 75, 637-647). For example, Han and coworkers in 2009 combined different receptor-G protein fusions and various mutant receptors to demonstrate allosteric modulation within a dopamine homodimer (Han, Y. et al. Nat. Chem. Biol. 2009, 5, 688-695). They reported that the D2 dopamine receptor homodimers are maximally activated upon a single agonist binding a single protomer in the dimer pair. When a second agonist binds the second protomer, it blunts the signal. If an inverse agonist binds the second protomer, it enhances the signal beyond agonist alone (Han, Y. et al. Nat. Chem. Biol. 2009, 5, 688-695).

In a different strategy, Teitler and coworkers developed pseudo-irreversible inactivators and reactivators that can be used to block only one of the protomers within the dimer pair in order to demonstrate the crosstalk within wild type serotonin homodimers (Teitler, M. et al. Pharmacol. Ther. 2012, 133, 205-217). This approach can and has been used to demonstrate the allosteric regulation within homodimers in native tissue samples. Application of this technique in vivo would be difficult given the multiple dosing regimen necessary and, therefore, would have very limited therapeutic applications (Teitler, M. et al. Pharmacol. Ther. 2012, 133, 205-217). Although these reports provide critical proof of the relevancy and functional significance of asymmetric signaling homodimers, the techniques employed are limited by their use of receptor mutations or subtle pharmacological differences that make adaption of the approaches to in vivo applications difficult and therapeutic applications inexecutable. Ideally, a pharmacological approach is needed to target and exploit allosteric communication between homodimers with a single chemical entity that could be used to examine the in vivo effects of asymmetric GPCR homodimers to study their potential as therapeutic targets.

One approach to pharmacologically targeting GPCR dimers is utilizing bivalent ligands. This approach was pioneered Portoghese and coworkers targeting the opioid receptors (Portoghese, P. S. et al. Life Sci. 1982, 31, 1283-1286 and Erez, M. et al. J. Med. Chem. 1982, 25, 847-849). Heterobivalent ligands featuring pharmacophores for two different receptor types have been utilized to exploit allosteric interactions within heterodimers to develop ligands with novel pharmacological profiles, tissue selectivity, and different functional effects (Daniels, D. J. et al. Proc. Natl. Acad. Sci. U. S. A. 2005, 102, 19208-19213; Smeester, B. A. et al. Eur. J. Pharmacol. 2014, 743, 48-52; Le Naour, M. et al. J. Med. Chem. 2013, 56, 5505-5513; Akgün, E. et al. J. Med. Chem. 2015, 58, 8647-8657, Le Naour, M. et al. J. Med. Chem. 2014, 57, 6383-6392 and Hiller, C. et al. J. Med. Chem. 2013, 56, 6542-6559). However, no one has exploited the allosteric communication that may occur between homodimers with bivalent ligands to produce novel pharmacologies. Unmatched bivalent ligands (UmBLs) have an agonist pharmacophore on one side of the bivalent ligand connected to an antagonist pharmacophore through an inert linker. The term UmBLs is used to separate this class of ligands from heterobivalent ligands that also have different pharmacophores on each side of the bivalent ligand, but are usually used to target different receptor types. This UmBL design has been proposed and reported previously, however, it has not been used to successfully exploit asymmetric signaling of GPCR homodimers (Kuhhorn, J. et al. J. Med. Chem. 2011, 54, 7911-7919; Fernandes, S. M. et al. Bioorg. Med. Chem. 2014, 22, 6360-6365 and Smith, N. J. et al. Pharmacol. Rev. 2010, 62, 701-725).

Both agonist and antagonist homobivalent ligands targeting the melanocortin receptor system have been previously reported (Lensing, C. J. et al. J. Med. Chem. 2016, 59, 3112-3128 and Lensing, C. J. et al. ACS Chem. Neurosci. 2017, 8, 1262-1278). Ligands targeting the melanocortin system have been implicated as potential therapeutics or used as pharmacological probes for a wide range of diseases states including cancer (Xu, L. P. et al. Proc. Natl. Acad. Sci. U. S. A. 2012, 109, 21295-21300; Josan, J. S. et al. Bioconjugate Chem. 2011, 22, 1270-1278; Barkey, N. M. et al. J. Med. Chem. 2011, 54, 8078-8084; Brabez, N. et al. ACS Med. Chem. Lett. 2013, 4, 98-102 and Brabez, N. et al. J. Med. Chem. 2011, 54, 7375-7384), skin pigmentation disorders (Langendonk, J. G. et al. N. Engl. J. Med. 2015, 373, 48-59), social disorders (Penagarikano, O. et al. Sci. Transl. Med. 2015, 7, 271 and Barrett, C. E. et al. Neuropharmacology 2014, 85, 357-366), sexual function disorders (Uckert, S. et al. Expert Opin. Invest. Drugs 2014, 23, 1477-1483; Clayton, A. H. et al. Women's Health 2016, 12, 325-337 and Kingsberg, S. et al. J. Sex. Med. 2015, 12, 389-389), Alzheimer's disease (Giuliani, D. et al. Mol. Cell. Neurosci. 2015, 67, 13-21 and Giuliani, D. et al. Neurobiol. Aging 2014, 35, 537-547), cachexia (Joppa, M. A. et al. Peptides 2005, 26, 2294-2301; Deboer, M. D. et al. Trends Endocrinol. Metab. 2006, 17, 199-204; Doering, S. R. et al. ACS Med. Chem. Lett. 2015, 6, 123-127 and Ericson, M. D. et al. J. Med. Chem. 2015, 58, 4638-4647), and obesity (Lensing, C. J. et al. J. Med. Chem. 2016, 59, 3112-3128; Irani, B. G. et al. Eur. J. Pharmacol. 2011, 660, 80-87; Marsh, D. J. et al. Nat. Genet. 1999, 21, 119-122 and Fan, W. et al. Nature 1997, 385, 165-168). All five melanocortin receptor subtypes (MC1-5R) signal through the Gₐₛ protein signaling pathway. In this pathway, agonist binding to the GPCR activates cAMP signal transduction pathways and also results in the recruitment of β-arrestin (Shinyama, H. et al. Endocrinology 2003, 144, 1301-1314). The melanocortin-3 receptor (MC3R) and melanocortin-4 receptor (MC4R) in particular have been elucidated to play a roles in energy homeostasis (Irani, B. G. et al. Eur. J. Pharmacol. 2011, 660, 80-87; Marsh, D. J. et al. Nat. Genet. 1999, 21, 119-122; Fan, W. et al. Nature 1997, 385, 165-168; Huszar, D. et al. Cell 1997, 88, 131-141 and Chen, A. S. et al. Nat. Genet. 2000, 26, 97-102). Ligands for the MC4R were under intense clinical development to treat obesity and related metabolic disorders; however these ligands were reported to have undesirable effects such as increasing blood pressure (Greenfield, J. R. et al. N. Engl. J. Med. 2009, 360, 44-52) or inducing male erections (Van der Ploeg, L. H. et al. Proc. Natl. Acad. Sci. U. S. A. 2002, 99, 11381-11386). It is hypothesized that ligands that target melanocortin homodimers may have unique effects from the current monovalent approaches, and may, therefore circumvent some side effects.

It is previously shown that an agonist homobivalent ligand produces a distinct in vivo pharmacological profile compared to monovalent counterpart suggesting that targeting putative melanocortin dimers may have physiological relevancy (Lensing, C. J. et al. ACS Chem. Neurosci. 2017, 8, 1262-1278). Furthermore, biased ligands would be valuable pharmacological probes to elucidate which signaling pathway is responsible for the various melanocortin dependent effects (i.e. lowered food intake vs increased blood pressure).

Currently, there is a need for new ligands that can asymmetrically signal the melanocortin receptor homodimers.

### SUMMARY OF THE INVENTION

This invention provides new ligands that are capable of signaling a melanocortin receptor homodimer. Accordingly, the invention provides a compound of formula I:

Y—X—Z I

or a salt thereof, wherein:

X is a linking group; and

Y is a melanocortin receptor agonist and Z is a melanocortin receptor antagonist; or Y is a melanocortin receptor antagonist and Z is a melanocortin agonist.

The invention also provides a pharmaceutical composition comprising a compound of formula I or a pharmaceutically acceptable salt thereof, and a pharmaceutically acceptable carrier.

The invention also provides a method for treating obesity or a disease associated with obesity in an animal (e.g., a mammal, such as a human) comprising administering a compound of formula I or a pharmaceutically acceptable salt thereof to the animal.

The invention also provides a compound of formula I or a pharmaceutically acceptable salt thereof for use in medical therapy.

The invention also provides a compound of formula I or a pharmaceutically acceptable salt thereof for the prophylactic or therapeutic treatment of obesity or a disease associated with obesity.

The invention also provides the use of a compound of formula I or a pharmaceutically acceptable salt thereof to prepare a medicament for treating obesity or a disease associated with obesity.

The invention also provides processes and intermediates disclosed herein that are useful for preparing a compound of formula I or a salt thereof.

### BRIEF DESCRIPTION OF THE FIGURES

FIGS. 1A-1C show hypothesized interaction of ligands with asymmetrically signaling melanocortin homodimers. FIG. 1A) Monovalent agonist ligands could occupy both receptors and result in both cAMP signaling and β-arrestin recruitment. FIG. 1B) Agonist homobivalent ligands could result in similar functional cAMP assays as monomeric ligands in spite of increased binding affinities due to asymmetric signaling. FIG. 1C) The working paradigm herein in which biased unmatched bivalent ligands (BUmBLs) containing an agonist pharmacophore and antagonist pharmacophore are postulated to result in biased signaling by agonizing one signaling pathway while antagonizing the other pathway when bound to the asymmetrically signaling homodimer.

FIGS. 2A-2D illustrate the in vitro functional pharmacology of MUmBLs at the MC4R. FIG. 2A) The cAMP signaling potency at the hMC4R was determined by AlphaScreen® assays. FIG. 2B) and FIG. 2C) The β-arrestin recruitment potency at the hMC4R was determined by PRESTO-Tango assays (Kroeze, W. K. et al. Nat. Struct. Mol. Biol. 2015, 22, 362-369). Functional cAMP data was normalized as discussed in experimental section to show tradition dose response curve with increasing response at increasing agonist concentration. FIG. 2D) Ligand induced response on bioluminescence resonance energy transfer (BRET) signal using the mMC4R-NanoLuc and mMC4R-HaloTag homodimer. Maximal BRET signal (100%) was defined as the signal measured when assay buffer (represented as A) was added. Each ligand was dosed at 10⁻⁵, 10⁻⁷ and 10⁻⁹M. Significance was determined using a one-way ANOVA to determine overall significance upon treatment followed by a Bonferroni post-hoc test to compare each ligand concentration to assay buffer control (A). * p<0.05, ** p=0.01, *** p<0.001. Data shown as the mean±standard error of the mean (SEM) determined from three independent experiments.

FIG. 3 illustrates a previously reported model for allosteric interactions in GPCR dimers. (Durroux 2005, Casdao 2007) (Durroux, T. Trends Pharmacol. Sci. 2005, 26, 376-384 and Casado, V. et al. Pharmacol. Ther. 2007, 116, 343-354). In this model, GPCRs oscillate through different conformational states. Different conformations have different propensity to signal through cAMP or through β-arrestin. Signaling is represented by arrows (states B, C, E, F, H, I, L). Conformational changes are represented based on receptor highlighting (states B, C, D, E, F, H, I, L). The binding of an agonist pharmacophore to one receptor that signals through cAMP stabilizes the second receptor's conformation to increases its propensity to signal through the β-arrestin recruitment pathway (State E). Therefore, the second agonist binding event results in β-arrestin recruitment (State F). The BUmBL design strategy can be used to block the β-arrestin recruitment by increasing the likelihood of an antagonist pharmacophore binding the second receptor in the homodimer (States G-I). Even if the opposite binding order occurs, the antagonist blocks β-arrestin recruitment since it is already bound to the receptor after the agonist induces a conformational change (States J-L). This models assumes that the receptors are dimeric in nature, but they are likely in an equilibrium as monomers and higher order oligomers (Durroux, T. Trends Pharmacol. Sci. 2005, 26, 376-384; Casado, V. et al. Pharmacol. Ther. 2007, 116, 343-354 and Tabor, A. et al. Sci. Rep. 2016, 6, 33233). This models also assumes that the bivalent synergistic binding mode is favored with MUmBLs due to the decreased entropic cost of binding of the second pharmacophore (Lensing, C. J. et al. J. Med. Chem. 2016, 59, 3112-3128).

FIGS. 4A-4B show the dose response effect of CJL-5-58 administered ICV on cumulative food intake (FIG. 4A) and change in body weight (FIG. 4B) in male wild type mice utilizing a fasting refeeding paradigm. The littermate age matched mice were fasted for 22 hours. Data is shown as mean±SEM. Data was analyzed using the SPSS (v23, IBM) using a multivariate general linear model followed by a Bonferroni's post hoc test. *p<0.05, ** p=0.01, *** p<0.001 for CJL-5-58 compared to saline.

FIGS. 5A-5D show the dose response effect of CJL-5-58 administered ICV on cumulative food intake in male and female wild type mice utilizing a fasting refeeding paradigm. The littermate age matched mice were fasted for 22 hours prior to treatment and the reintroduction of food. Data is shown as mean±SEM. Data was analyzed using the SPSS (v23, IBM) using a multivariate general linear model followed by a Bonferroni's post hoc test. *p<0.05, ** p=0.01, *** p<0.001 for CJL-5-58 compared to saline. FIG. 5A Male cumulative fasting food intake; FIG. 5B Female cumulative fasting food intake; FIG. 5C Male cumulative fasting food intake; and FIG. 5D Female cumulative fasting food intake. For body weight information see FIG. 6.

FIGS. 6A-6B show the dose response effect of CJL-5-58 administered ICV on change in body weight in male and female wild type mice utilizing a fasting refeeding paradigm. Data is shown as mean±SEM. Data was analyzed using the SPSS (v23, IBM). *p<0.05, ** p=0.01, *** p<0.001 for CJL-5-58 compared to saline. FIG. 6A Male fasting change in weight; and FIG. 6B Female fasting change in weight. For food intake information see FIG. 5.

FIGS. 7A-7D show the effect of CJL-5-58 administered ICV on cumulative food intake in male and female wild type mice utilizing nocturnal feeding paradigm. Satiated mice were treated 2 hours prior to lights out. Data is shown as mean±SEM. Data was analyzed using the SPSS (v23, IBM) using a multivariate general linear model followed by a Bonferroni's post hoc test.. *p<0.05, ** p=0.01, *** p<0.001 for CJL-5-58 compared to saline. FIG. 7A Male cumulative nocturnal food intake; FIG. 7B Female cumulative nocturnal food intake; FIG. 7C Male cumulative nocturnal food intake; and FIG. 7D Female cumulative nocturnal food intake.

FIGS. 8A-8B shows the effect of CJL-5-58 administered ICV on change in body weight (g) in male and female wild type mice utilizing nocturnal feeding paradigm. Data is shown as mean±SEM. Data was analyzed using the SPSS (v23, IBM). *p<0.05, ** p=0.01, *** p<0.001 for CJL-5-58 compared to saline. FIG. 8A Male nocturnal mouse weight; and FIG. 8B Female nocturnal mouse weight.

FIGS. 9A-9D show the TSE metabolic cage parameters after ICV administration of 5 nmols of CJL-5-58, or a combination of 5 nmols CJL-1-14 and 5 nmols CJL-1-80 (10 nmols total combined peptide) to male wild type mice in a fasting-refeeding paradigm. The littermate age matched mice were fasted for 22 hours. Data is shown as mean±SEM. Data was analyzed using the SPSS (v23, IBM) using a multivariate general linear model followed by a Bonferroni's post hoc test. *p<0.05, ** p=0.01, *** p<0.001 for CJL-5-58 compared to saline. %p<0.05, %% p=0.01, %%% p<0.001 for saline compared to co-administration of CJL-1-14 and CJL-1-80. +p<0.05, ++ p=0.01, +++ p<0.001 for CJL-5-58 compared to co-administration of CJL-1-14 and CJL-1-80. FIG. 9A Cumulative fasting food intake; FIG. 9B Average hourly fasting RER; FIG. 9C Cumulative fasting water intake; and FIG. 9D Fasting energy expenditure. For all parameters from −18 to 24 hours, see FIG. 10.

FIGS. 10A-10E show the TSE metabolic cage parameters after ICV administration of 5 nmols of CJL-5-58, or a combination of CJL-1-14 and CJL-1-80 (10 nmols total peptide) to male wild type mice in a fasting refeeding paradigm. The littermate age matched mice were fasted for 22 hours. Data is shown as mean±SEM. Data was analyzed using the SPSS (v23, IBM). *p<0.05, ** p=0.01, *** p<0.001 for CJL-5-58 compared to saline. %p<0.05, %% p=0.01, %%% p<0.001 for saline compared to co-administration of CJL-1-14 and CJL-1-80. +p<0.05, ++ p=0.01, +++ p<0.001 for CJL-5-58 compared to co-administration of CJL-1-14 and CJL-1-80. FIG. 10A Cumulative fasting food intake; FIG. 10B Average hourly fasting RER; FIG. 10C Cumulative fasting water intake; FIG. 10D Fasting energy expenditure; and FIG. 10E Average hourly fasting activity.

FIGS. 11A-11D show the TSE metabolic cage parameters after ICV administration of 5 nmols of CJL-5-58, or a combination of CJL-1-14 and CJL-1-80 (10 nmols total peptide) to male wild type mice in a nocturnal feeding paradigm (no fasting). Satiated mice were treated 2 hours prior to lights out. Data is shown as mean±SEM. Data was analyzed using the SPSS (v23, IBM) using a multivariate general linear model followed by a Bonferroni's post hoc test. *p<0.05, ** p=0.01, *** p<0.001 for CJL-5-58 compared to saline. %p<0.05, %% p=0.01, *** p<0.001 for saline compared to co-administration of CJL-1-14 and CJL-1-80. FIG. 11A Cumulative nocturnal food intake; FIG. 11B Average hourly nocturnal RER; FIG. 11C Cumulative nocturnal water intake; and FIG. 11D Nocturnal energy expenditure. For all parameters from 0 to 24 hours, see FIG. 12.

FIGS. 12A-12E show the TSE metabolic cage parameters after ICV administration of 5 nmols of CJL-5-58, or a combination of CJL-1-14 and CJL-1-80 (10 nmols total peptide) to male wild type mice in a nocturnal feeding paradigm. Data is shown as mean±SEM. Data was analyzed using the SPSS (v23, IBM). *p<0.05, ** p=0.01, *** p<0.001 for CJL-5-58 compared to saline. %p<0.05, %% p=0.01, %%% p<0.001 for saline compared to co-administration of CJL-1-14 and CJL-1-80. + p<0.05, ++ p=0.01, +++p<0.001 for CJL-5-58 compared to co-administration of CJL-1-14 and CJL-1-80. FIG. 12A Cumulative nocturnal food intake; FIG. 12B Average hourly nocturnal RER; FIG. 12C Cumulative nocturnal water intake; FIG. 12D Nocturnal energy expenditure; and FIG. 12E Average hourly nocturnal activity.

FIG. 13 shows the TSE metabolic cage parameters after ICV administration of 5.0, 2.5, or 1.0 nmols of CJL-5-58 to male MC3RKO mice in a nocturnal feeding paradigm. Data is shown as mean±SEM. Data was analyzed using the SPSS (v23, IBM). *p<0.05, ** p=0.01, *** p<0.001 for saline compared to 5 nmols CJL-5-58. +p<0.05, ++ p=0.01, +++ p<0.001 for saline compared 2.5 nmol CJL-5-58. Note: Some toxicity was observed.

FIG. 14 shows the TSE metabolic cage parameters after ICV administration of 1.0 or 0.5 nmols of CJL-5-58 to male MC3RKO mice in a fasting-refeeding paradigm. Data is shown as mean±SEM. Data was analyzed using the SPSS (v23, IBM). *p<0.05, ** p=0.01, *** p<0.001 for saline compared to 1.0 nmols CJL-5-58. +p<0.05, ++ p=0.01, +++ p<0.001 for saline compared 0.5 nmol CJL-5-58. Note: Some toxicity was observed.

FIG. 15 shows the effect of CJL-5-58 (5 nmols) administered ICV on cumulative food intake and body weight in male MC4RKO mice utilizing nocturnal feeding and fasting-refeeding paradigm in conventional cages. Data is shown as mean±SEM. Data was analyzed using the SPSS (v23, IBM). *p<0.05, ** p=0.01, *** p<0.001 for 5 nmol CJL-5-58 compared to saline. Note: Some toxicity was observed.

FIG. 16 shows the TSE metabolic cage parameters after ICV administration of 5.0 or 2.5 nmols of CJL-1-124 to male wild type mice in a nocturnal feeding paradigm. Data is shown as mean±SEM. Data was analyzed using the SPSS (v23, IBM). *p<0.05, ** p=0.01, *** p<0.001 for saline compared to 5 nmols CJL-5-58. %p<0.05, %% p=0.01, %%% p<0.001 for saline compared 2.5 nmol CJL-5-58. Note: Some toxicity was observed.

FIG. 17 shows the TSE metabolic cage parameters after ICV administration of 5.0 or 2.5 nmols of CJL-1-124 to male MC3RKO mice in a nocturnal feeding paradigm. Data is shown as mean±SEM. Data was analyzed using the SPSS (v23, IBM). *p<0.05, ** p=0.01, *** p<0.001 for saline compared to 5 nmols CJL-5-58. %p<0.05, %% p=0.01, %%% p<0.001 for saline compared 2.5 nmol CJL-5-58. Note: Some toxicity was observed.

FIG. 18 shows the effect of CJL-1-124 (5.0 and 2.5 nmols) administered ICV on cumulative food intake and body weight in male MC4RKO mice utilizing nocturnal feeding paradigm in conventional cages. Data is shown as mean±SEM. Data was analyzed using the SPSS (v23, IBM). %p<0.05 for 2.5 nmol CJL-5-58 compared to saline. Note: Some toxicity was observed.

FIG. 19 shows Table 5: adverse reactions observed in the current experiments with CJL-5-58 in wild-type mice, MC3RKO mice, and MC4RKO mice. Adverse means that adverse reactions were observed in the experiments as described in the text. Sig. Effect means that a significant effect on food intake was observed. N.D. means not determined indicating that the experiment was not performed.

FIG. 20 shows Table 1: functional data at the hMC4R. The cAMP signaling potency was determined by AlphaScreen™ assays. The β-arrestin recruitment potency was determined by PRESTO-Tango assays (Kroeze, W. K. et al. Nat. Struct. Mol. Biol. 2015, 22, 362-369). The reported errors are the standard error of the mean (SEM) determined from at least three independent experiments. Changes less than 3-fold were considered to be within the inherent experimental assay error. The % symbol represents amount of maximal signal observed at 10 μM compared to control NDP-MSH maximal signal.

FIG. 21 shows Table 2: analytical data for peptides synthesized. HPLC k′=(peptide retention time−solvent retention time)/solvent retention time. System 1 is a 10% to 90% gradient of acetonitrile in water containing 0.1% trifluoroacetic acid over 35 minutes at a flow rate of 1.5 mL/min, and system 2 is the same but with methanol replacing acetonitrile. Product purity was determined by HPLC purity in the solvent system which showed the least purity and integrating the area under the curves of the chromatograms collected at 214 nm. Mass observed was calculated from the M+1 or (M+2)/2 peak.

FIG. 22 shows Table 3: analytical data for additional ligands

FIG. 23 shows Table 4: functional data at the mMC1R, mMC3R, mMC4R, and mMC5R. The cAMP signaling potency was determined by AlphaScreen® assays. The reported errors are the standard error of the mean (SEM) determined from at least three independent experiments. Changes less than 3-fold were considered to be within the inherent experimental assay error. NS means compound was not soluble in bioassay compatible solvent. PA means partial agonism was observed.

FIG. 24 shows Table 5: summary of competitive binding experiments with 1251-NDP-MSH at the mMC1R, mMC3R, and mMC4R. IC₅₀ values were determined by competitive binding in which experimental compounds were used to displace ¹²⁵I-NDP-MSH in a dose-response manner. In competitive experiments, % represent the amount of ¹²⁵I-NDP-MSH signal reduction at 100 μM. The reported errors are the standard error of the mean (SEM). Changes less than 3-fold were considered to be within the inherent experimental assay error. NA means no activity observed up to 100 μM. NA means no displacement was observed at 100 μM. NS means compound was not soluble in bioassay compatible solvent.

FIG. 25 shows chemical structures of selected scaffolds and linkers used.

### DETAILED DESCRIPTION

The term “alkyl”, by itself or as part of another substituent, means, unless otherwise stated, a straight or branched chain hydrocarbon radical, having the number of carbon atoms designated (i.e., C₁₋₄ means one to four carbons). Non limiting examples of “alkyl” include methyl, ethyl, propyl, isopropyl, butyl, iso-butyl, sec-butyl.

The term “halo” means fluoro, chloro, bromo, or iodo.

The term “haloalkyl” means an alkyl that is optionally substituted with one or more (e.g., 1, 2, 3, 4, or 5) halo. Non limiting examples of “haloalkyl” include iodomethyl, bromomethyl, chloromethyl, fluoromethyl, trifluoromethyl, 2-chloroethyl, 2-fluoroethyl, 2,2,2-trifluoroethyl 2,2-difluoroethyl and pentafluoroethyl.

The term “alkoxy” refers to an alkyl groups attached to the remainder of the molecule via an oxygen atom (“oxy”).

The term “alkylthio” refers to an alkyl groups attached to the remainder of the molecule via a thio group.

The term “cycloalkyl” refers to a saturated all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “alkoxycarbonyl” as used herein refers to a group (alkyl)-O—C(═O)—, wherein the term alkyl has the meaning defined herein.

The term “alkanoyloxy” as used herein refers to a group (alkyl)-C(═O)—O—, wherein the term alkyl has the meaning defined herein.

As used herein, the term “heteroatom” is meant to include oxygen (O), nitrogen (N), sulfur (S) and silicon (Si).

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; and (C₂-C₆)alkanoyloxy can be acetoxy, propanoyloxy, butanoyloxy, isobutanoyloxy, pentanoyloxy, or hexanoyloxy.

As used herein a wavy line “ [symbol] ” that intersects a bond in a chemical structure indicates the point of attachment of the bond that the wavy bond intersects in the chemical structure to the remainder of a molecule.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. Dap, PyrAla, ThiAla, (pCl)Phe, (pNO₂)Phe, ε-Aminocaproic acid, Met[O₂], dehydPro, (31)Tyr, norleucine (Nle), para-I-phenylalanine ((pI)Phe), 2-napthylalanine (2-Nal), β-cyclohexylalanine (Cha), β-alanine β-Ala), phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid (Tic), penicillamine, ornithine, citruline, a-methyl-alanine, para-b enzoylphenylalanine, phenylglycine, propargylglycine, sarcosine, and tert-butylglycine) in D or L form. The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an a-methylbenzyl amide). Other suitable amino and carboxy protecting groups are known to those skilled in the art (See for example, T. W. Greene, Protecting Groups In Organic Synthesis; Wiley: New York, 1981, and references cited therein). An amino acid can be linked to the remainder of a compound of formula I through the carboxy terminus, the amino terminus, or through any other convenient point of attachment, such as, for example, through the sulfur of cysteine.

As used herein, the term “residue of an amino acid” means a portion of an amino acid. Non-limiting examples include a residue of L-histidine, D-histidine, L-phenylalanine, D-phenylalanine, L-arginine, D-arginine, L-tryptophan, D-tryptophan, L-2-naphthyl-alanine, and D-2-naphthyl-alanine, wherein certain atoms (e.g., H or OH) may have been removed to link the amino acids via a peptide bond.

It is understood that Y and Z can be linked to X at any synthetically feasible position on Y or Z.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound of formula I herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95% the absolute stereoisomer depicted. In another embodiment, the compound may be at least 99% the absolute stereoisomer depicted.

### Linker

As described herein, the one portion of a compound can be bonded (connected) to the remainder of the compound through an optional linker. In one embodiment the linker is absent. The linker can vary in length and atom composition and for example can be branched or non-branched or cyclic or a combination thereof. The linker may also modulate the properties of the compound such as but not limited to solubility, stability and aggregation.

In one embodiment the linker comprises about 3-100 atoms. In one embodiment the linker comprises about 3-50 atoms. In one embodiment the linker comprises about 3-25 atoms.

In one embodiment the linker comprises atoms selected from H, C, N, S and O.

In one embodiment the linker comprises atoms selected from H, C, N, S, P and O.

In one embodiment the linker comprises a branched or unbranched, saturated or unsaturated, hydrocarbon chain, having from about 1 to 100 (or 1-50, 1-25, 1-10, 1-5, 5-100, 5-50, 5-25, 5-10 or 2-5 carbon atoms) wherein one or more of the carbon atoms is optionally replaced independently by —O—, —S, —N(R^{a})—, 3-7 membered heterocycle, 5-6-membered heteroaryl or carbocycle and wherein each chain, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy, wherein each R^{a} is independently H or (C₁-C₆)alkyl. In one embodiment the linker comprises a branched or unbranched, saturated or unsaturated, hydrocarbon chain, having from about 1 to 100 (or 1-50, 1-25, 1-10, 1-5, 5-100, 5-50, 5-25, 5-10 or 2-5 carbon atoms) wherein one or more of the carbon atoms is optionally replaced independently by —O—, —S, —N(R^{a})—, wherein each R^{a} is independently H or (C₁-C₆)alkyl.

In one embodiment the linker comprises a polyethylene glycol. In one embodiment the linker comprises a polyethylene glycol linked to the remainder of the targeted conjugate by a carbonyl group. In one embodiment the polyethylene glycol comprises about 1 to about 10 (e.g., —CH₂CH₂O—) units (Greenwald, R. B., et al., Poly (ethylene glycol) Prodrugs: Altered Pharmacokinetics and Pharmacodynamics, Chapter, 2.3.1., 283-338; Filpula, D., et al., Releasable PEGylation of proteins with customized linkers, Advanced Drug Delivery, 60, 2008, 29-49; Zhao, H., et al., Drug Conjugates with Poly(Ethylene Glycol), Drug Delivery in Oncology, 2012, 627-656).

In one embodiment the linker is —NH(CH₂CH₂O)₄CH₂CH₂C(═O)—. In one embodiment the linker is —NH(CH₂CH₂O)ₙCH₂CH₂C(═O)— wherein n is 1-10, 1-5, 2-10, 2-5, 3-10, 3-5, 4-10, 4-5. In one embodiment the linker is —(CH₂CH₂O)₄CH₂CH₂C(═O)—.

The terms “treat” and “treatment” refer to both therapeutic treatment and prophylactic or preventative measures, wherein the object is to prevent or decrease an undesired physiological change or disorder, such as a metabolic disorder (e.g., obesity) or a disease associated with the metabolic disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

In one embodiment, the melanocortin receptor agonist comprises an amino acid sequence of His-DPhe-Arg-Trp (SEQ ID NO:1).

In one embodiment, the melanocortin receptor antagonist comprises an amino acid sequence of His-DNal(2′)-Arg-Trp (SEQ ID NO:2).

In one embodiment, the compound of invention has the following formula II:

CH₃C(═O)-A-X—B—NH₂ II

or a salt thereof, wherein:

A is -His-DPhe-Arg-Trp-, -His-DNal(2′)-Arg-Trp-, -DTrp-DArg-Phe-DHis-, or -DTrp-DArg-Nal(2′)-DHis-;

B is -His-DPhe-Arg-Trp-, -His-DNal(2′)-Arg-Trp-, -DTrp-DArg-Phe-DHis-, or -DTrp-DArg-Nal(2′)-DHis-;

X is a linking group;

His is a residue of L-histidine;

DHis is a residue of D-histidine;

Phe is a residue of L-phenylalanine, wherein the phenyl ring is optionally substituted with one or more groups selected from halo, (C₁-C₄)alkyl, —O(C₁-C₄)alkyl, (C₁-C₄)haloalkyl, or —O(C₁-C₄)haloalkyl;

DPhe is a residue of D-phenylalanine, wherein the phenyl ring is optionally substituted with one or more groups selected from halo, (C₁-C₄)alkyl, —O(C₁-C₄)alkyl, (C₁-C₄)haloalkyl, or —O(C₁-C₄)haloalkyl;

Arg is a residue of L-arginine;

DArg is a residue of D-arginine;

Trp is a residue of L-tryptophan, wherein the indolyl ring is optionally substituted with one or more groups selected from halo, (C₁-C₄)alkyl, —O(C₁-C₄)alkyl, (C₁-C₄)haloalkyl, or —O(C₁-C₄)haloalkyl;

DTrp is a residue of D-tryptophan, wherein the indolyl ring is optionally substituted with one or more groups selected from halo, (C₁-C₄)alkyl, —O(C₁-C₄)alkyl, (C₁-C₄)haloalkyl, or —O(C₁-C₄)haloalkyl;

Nal(2′) is a residue of L-2-naphthyl-alanine, wherein the phenyl ring is optionally substituted with one or more groups selected from halo, (C₁-C₄)alkyl, —O(C₁-C₄)alkyl, (C₁-C₄)haloalkyl, or —O(C₁-C₄)haloalkyl; and

DNal(2′) is a residue of D-2-naphthyl-alanine, wherein the phenyl ring is optionally substituted with one or more groups selected from halo, (C₁-C₄)alkyl, —O(C₁-C₄)alkyl, (C₁-C₄)haloalkyl, or —O(C₁-C₄)haloalkyl;

provided if A is -His-DPhe-Arg-Trp-, B is not -His-DPhe-Arg-Trp-, wherein the phenyl ring and the indolyl ring are not substituted;

and provided if A is -His-DNal(2′)-Arg-Trp-, B is not -His-DNal(2′)-Arg-Trp-, wherein the naphthyl ring and the indolyl ring are not substituted.

In one embodiment, A is -His-DPhe-Arg-Trp- or -His-DNal(2′)-Arg-Trp-.

In one embodiment, A is:

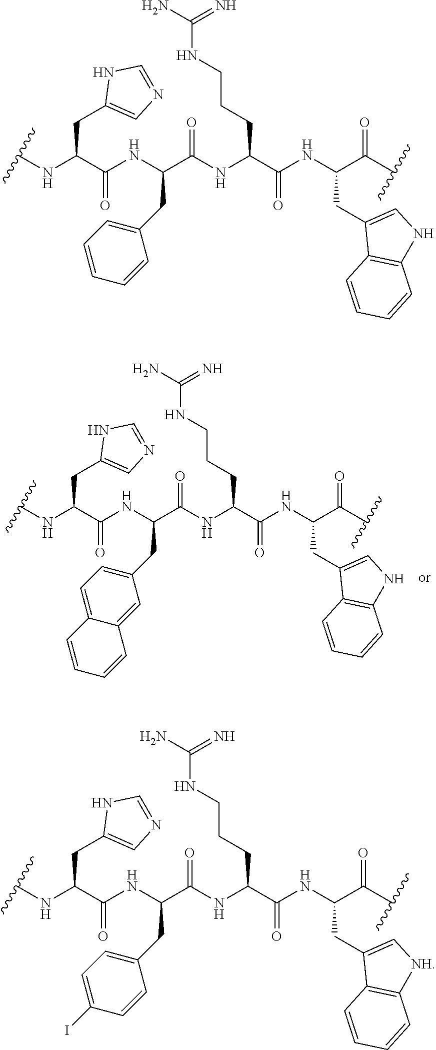 [SMILES: *N[C@@H](Cc1cnc[nH]1)C(=O)N[C@H](Cc1ccccc1)C(=O)N[C@@H](CCCNC(=N)N)C(=O)N[C@@H](Cc1c[nH]c2ccccc12)C(*)=O ; *N[C@@H](Cc1cnc[nH]1)C(=O)N[C@H](Cc1ccc2ccccc2c1)C(=O)N[C@@H](CCCNC(=N)N)C(=O)N[C@@H](Cc1c[nH]c2ccccc12)C(*)=O ; "or" ; *N[C@@H](Cc1cnc[nH]1)C(=O)N[C@H](Cc1ccc(I)cc1)C(=O)N[C@@H](CCCNC(=N)N)C(=O)N[C@@H](Cc1c[nH]c2ccccc12)C(*)=O]

In one embodiment, B is -His-DPhe-Arg-Trp- or -His-DNal(2′)-Arg-Trp-.

In one embodiment, B is:

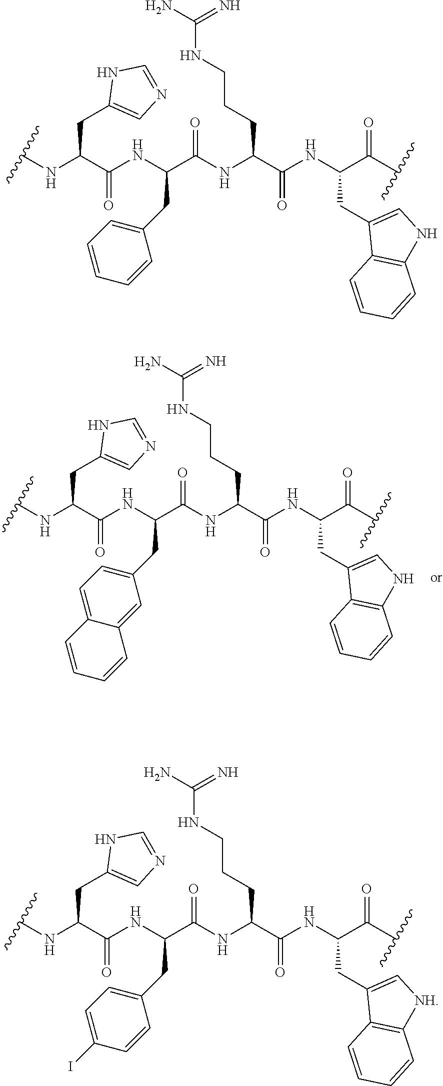 [SMILES: *N[C@@H](Cc1cnc[nH]1)C(=O)N[C@H](Cc1ccccc1)C(=O)N[C@@H](CCCNC(=N)N)C(=O)N[C@@H](Cc1c[nH]c2ccccc12)C(*)=O ; *N[C@@H](Cc1cnc[nH]1)C(=O)N[C@H](Cc1ccc2ccccc2c1)C(=O)N[C@@H](CCCNC(=N)N)C(=O)N[C@@H](Cc1c[nH]c2ccccc12)C(*)=O ; "or" ; *N[C@@H](Cc1cnc[nH]1)C(=O)N[C@H](Cc1ccc(I)cc1)C(=O)N[C@@H](CCCNC(=N)N)C(=O)N[C@@H](Cc1c[nH]c2ccccc12)C(*)=O]

In one embodiment, the compound of invention is a compound of formula Ia:

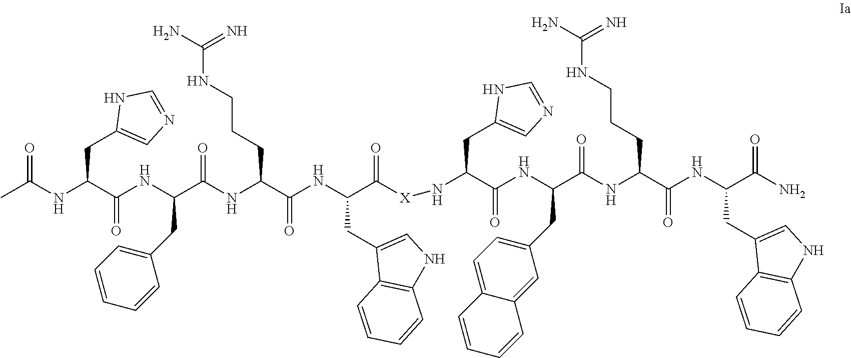

or a salt thereof.

In one embodiment, the compound of invention is a compound of formula Ib:

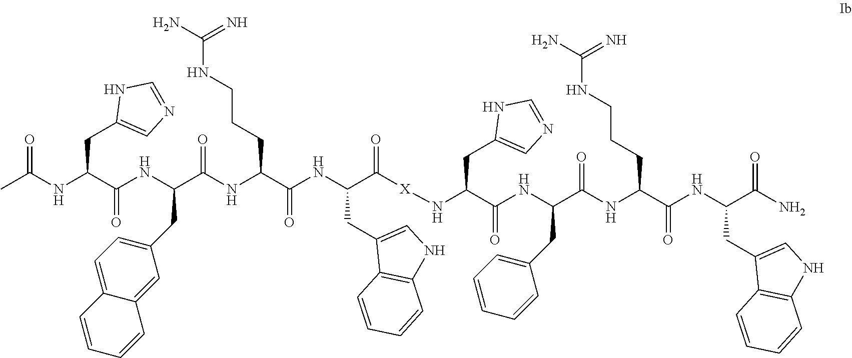

or a salt thereof.

In one embodiment, X is a branched or unbranched, saturated or unsaturated, hydrocarbon chain, having from about 10-100 carbon atoms, wherein one or more of the carbon atoms is optionally replaced independently by —O—, —S, —N(R^{a})—, or 3-7 membered heterocycle, wherein the hydrocarbon chain is optionally substituted with one or more substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), or carboxy, wherein each R^{a} is independently H or (C₁-C₆)alkyl.

In one embodiment, X is a branched or unbranched, saturated or unsaturated, hydrocarbon chain, having from about 10-50 carbon atoms, wherein one or more of the carbon atoms is optionally replaced independently by —O—, —S, —N(R^{a})—, or 3-7 membered heterocycle, wherein the hydrocarbon chain is optionally substituted with one or more substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), or carboxy, wherein each R^{a} is independently H or (C₁-C₆)alkyl.

In one embodiment, X comprises at least one unit of —CH₂CH₂O—.

In one embodiment, X comprises about 3 to 10 units of —CH₂CH₂O—.

In one embodiment, X is —NH(CH₂CH₂O)ₙCH₂CH₂C(═O)—, wherein n is 1, 2, 3, 4, 5, 6, 7, 8, 9, or 10.

In one embodiment, X is —NH(CH₂CH₂O)ₙCH₂CH₂C(═O)—, wherein n is 3, 4, 5, or 6.

In one embodiment, X is:

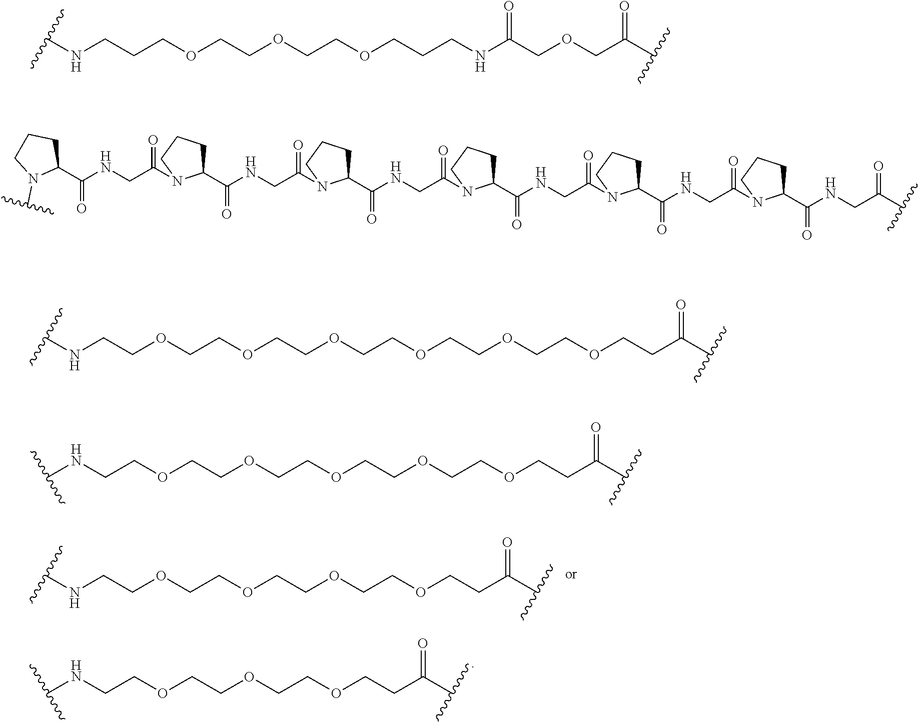

In one embodiment, X is:

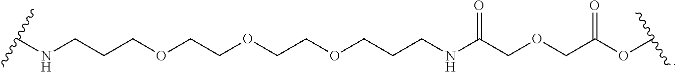 [SMILES: *NCCCOCCOCCOCCCNC(=O)COCC(=O)O*]

In one embodiment, the compound of invention is selected from the group consisting of:

Ac-His-DPhe(p-I)-Arg-Trp-(PEDG20)-His-DPhe(p-I)-Arg-Trp-NH₂;

Ac-His-DNal(2′)-Arg-Trp-(Pro-Gly)₆-His-DPhe-Arg-Trp-NH₂;

Ac-His-DPhe-Arg-Trp-(PEDG20)-His-DNal(2′)-Arg-Trp-NH₂;

Ac-His-DNal(2′)-Arg-Trp-(PEDG20)-His-DPhe-Arg-Trp-NH₂;

Ac-His-DNal(2′)-Arg-Trp-(PEDG20)-(PEDG20)-His-DPhe-Arg-Trp-NH₂;

Ac-His-DPhe(p-I)-Arg-Trp-(Pro-Gly)₆-His-DPhe-Arg-Trp-NH₂;

Ac-His-DPhe(p-I)-Arg-Trp-(PEDG20)-His-DPhe-Arg-Trp-NH₂;

Ac-His-DPhe-Arg-Trp-(PEDG20)-His-DPhe(p-I)-Arg-Trp-NH₂;

Ac-His-DNal(2′)-Arg-Trp-(PEDG20)-His-DPhe(p-I)-Arg-Trp-NH₂;

Ac-His-DPhe(p-I)-Arg-Trp-(PEDG20)-His-DNal(2′)-Arg-Trp-NH₂;

Ac-His-DPhe-Arg-Trp-(PEG)₂(22atoms)-His-DNal(2′)-Arg-Trp-NH₂;

Ac-His-DPhe-Arg-Trp-(PEDG20)-His-DNal(2′)-Arg-Trp-NH₂;

Ac-His-DPhe-Arg-Trp-(PEG)(19atoms)-His-DNal(2′)-Arg-Trp-NH₂;

Ac-DTrp-DArg-Phe-DHis-(PEG)(22 atoms)-His-DPhe-Arg-Trp-NH₂;

Ac-DTrp-DArg-Phe-DHis-(PEDG20)-His-DPhe-Arg-Trp-NH₂;

Ac-DTrp-DArg-Phe-DHis-(PEG)(19 atoms)-His-DPhe-Arg-Trp-NH₂;

Ac-His-DPhe-Arg-Trp-(PEG)(22 atoms)-DTrp-DArg-Phe-DHis-NH₂;

Ac-His-DPhe-Arg-Trp-(PEDG20)-DTrp-DArg-Phe-DHis-NH₂;

Ac-His-DPhe-Arg-Trp-(PEG)(19 atoms)-DTrp-DArg-Phe-DHis-NH₂;

Ac-DTrp-DArg-Phe-DHis-(PEG)(22 atoms)-DTrp-DArg-Phe-DHis-NH₂;

Ac-DTrp-DArg-Phe-DHis-(PEDG20)-DTrp-DArg-Phe-DHis-NH₂; and

Ac-DTrp-DArg-Phe-DHis-(PEG)(19 atoms)-DTrp-DArg-Phe-DHis-NH₂;

and salts thereof, wherein:

Ac is CH₃C(═O)—;

His is a residue of L-histidine;

DHis is a residue of D-histidine;

Phe is a residue of L-phenylalanine;

DPhe is a residue of D-phenylalanine;

Arg is a residue of L-arginine;

DArg is a residue of D-arginine;

Trp is a residue of L-tryptophan;

DTrp is a residue of D-tryptophan;

DNal(2′) is a residue of D-2-naphthyl-alanine;

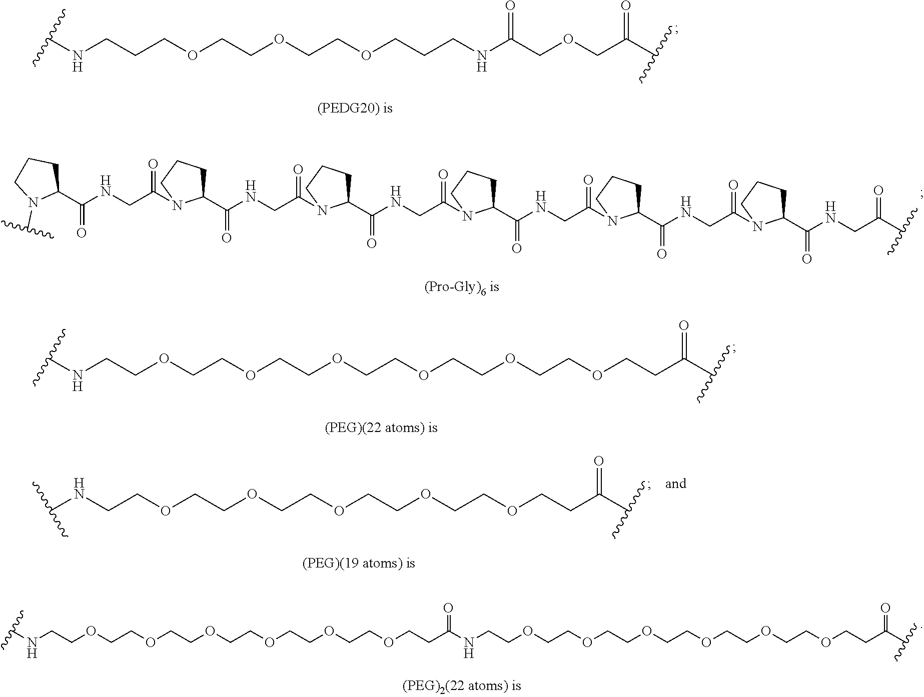 [SMILES: *NCCCOCCOCCOCCCNC(=O)COCC(*)=O ; *C(=O)CNC(=O)[C@@H]1CCCN1C(=O)CNC(=O)[C@@H]1CCCN1C(=O)CNC(=O)[C@@H]1CCCN1C(=O)CNC(=O)[C@@H]1CCCN1C(=O)CNC(=O)[C@@H]1CCCN1C(=O)CNC(=O)[C@@H]1CCCN1* ; *NCCOCCOCCOCCOCCOCCOCCC(*)=O ; *NCCOCCOCCOCCOCCOCCC(*)=O ; *NCCOCCOCCOCCOCCOCCOCCC(=O)NCCOCCOCCOCCOCCOCCOCCC(*)=O]

In one embodiment, the compound of invention is:

Ac-His-DNal(2′)-Arg-Trp-(Pro-Gly)₆-His-DPhe-Arg-Trp-NH₂;

Ac-His-DPhe-Arg-Trp-(PEDG20)-His-DNal(2′)-Arg-Trp-NH₂;

Ac-His-DNal(2′)-Arg-Trp-(PEDG20)-His-DPhe-Arg-Trp-NH₂; or

Ac-His-DNal(2′)-Arg-Trp-(PEDG20)-(PEDG20)-His-DPhe-Arg-Trp-NH₂;

or a salt thereof, wherein:

Ac is CH₃C(═)—;

His is a residue of L-histidine;

DPhe is a residue of D-phenylalanine;

Arg is a residue of L-arginine;

Trp is a residue of L-tryptophan;

DNal(2′) is a residue of D-2-naphthyl-alanine;

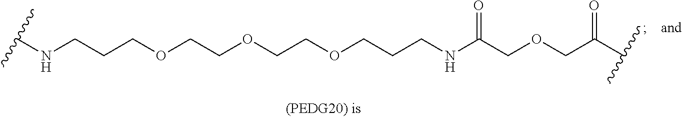 [SMILES: *NCCCOCCOCCOCCCNC(=O)COCC(*)=O]

In one embodiment the comnound of invention is:

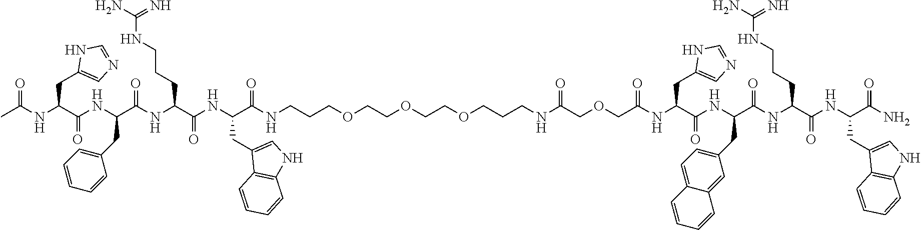 [SMILES: CC(=O)N[C@@H](Cc1cnc[nH]1)C(=O)N[C@H](Cc1ccccc1)C(=O)N[C@@H](CCCNC(=N)N)C(=O)N[C@@H](Cc1c[nH]c2ccccc12)C(=O)NCCCOCCOCCOCCCNC(=O)COCC(=O)N[C@@H](Cc1cnc[nH]1)C(=O)N[C@H](Cc1ccc2ccccc2c1)C(=O)N[C@@H](CCCNC(=N)N)C(=O)N[C@@H](Cc1c[nH]c2ccccc12)C(N)=O]

or a salt thereof.

In one embodiment, the compound of invention comprises first amino acid sequence having at least 80% sequence identity to His-DPhe-Arg-Trp (SEQ ID NO:1), and second amino acid sequence at least 80% identity to His-DNal(2′)-Arg-Trp (SEQ ID NO:2), or a salt thereof.

In one embodiment, the compound of invention comprises first amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:1, and second amino acid sequence at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:2, or a salt thereof.

In one embodiment, the compound of invention comprises first amino acid sequence having at least 90% sequence identity to SEQ ID NO:1, and second amino acid sequence at least 90% identity to SEQ ID NO:2, or a salt thereof.

In one embodiment, the compound of invention comprises first amino acid sequence having at least 99% sequence identity to SEQ ID NO:1, and second amino acid sequence at least 99% identity to SEQ ID NO:2, or a salt thereof.

In one embodiment, the compound of invention comprises first amino acid sequence of SEQ ID NO:1, and second amino acid sequence of SEQ ID NO:2, or a salt thereof.

In one embodiment, the compound of invention is an agonist for MC1R, MC3R, MC4R or MC5R.

In one embodiment, the compound of invention is a selective agonist for MC1R, MC3R, MC4R or MC5R.

One embodiment of the invention provides a dietary supplement comprising a compound of formula I, or a salt thereof.

Another embodiment of the invention provides a prodrug of a compound of formula I or a salt thereof. As used herein the term “prodrug” refers to a biologically inactive compound that can be metabolized in the body to produce a biologically active form of the compound.

In one embodiment, the disease associated with obesity is diabetes, cardiovascular disease or hypertension.

One embodiment of the invention provides a method of modulating (e.g., increasing or decreasing) the activity of a melanocortin receptor in vitro or in vivo comprising contacting the receptor with an effective amount of a compound of formula I, or a pharmaceutically acceptable salt thereof.

One embodiment of the invention provides a compound of formula I, or a pharmaceutically acceptable salt thereof for use in modulating (e.g., increasing or decreasing) the activity of a melanocortin receptor in vitro or in vivo.

One embodiment of the invention provides the use of a compound of formula I, or a pharmaceutically acceptable salt thereof for the manufacture of a medicament for modulating (e.g., increasing or decreasing) the activity of a melanocortin receptor in vitro or in vivo.

One embodiment of the invention provides a method of modulating (e.g. increasing or decreasing) the activity of a melanocortin receptor homodimer in vitro or in vivo comprising contacting the homodimer with an effective amount of a compound of formula I, or a pharmaceutically acceptable salt thereof.

One embodiment of the invention provides a compound of formula I, or a pharmaceutically acceptable salt thereof for use in modulating (e.g., increasing or decreasing) the activity of a melanocortin receptor homodimer in vitro or in vivo.

One embodiment of the invention provides the use of a compound of formula I, or a pharmaceutically acceptable salt thereof for the manufacture of a medicament for modulating (e.g., increasing or decreasing) the activity of a melanocortin receptor homodimer in vitro or in vivo.

One embodiment of the invention provides a method of activing cAMP signaling and simultaneously blocking β-arrestin recruitment in vitro or in vivo comprising contacting a melanocortin receptor homodimer with an effective amount of a compound of formula I, or a pharmaceutically acceptable salt thereof.

One embodiment of the invention provides a compound of formula I, or a pharmaceutically acceptable salt thereof, for use in activing cAMP signaling and simultaneously blocking β-arrestin recruitment in vitro or in vivo.

One embodiment of the invention provides the use of a compound of formula I, or a pharmaceutically acceptable salt thereof for manufacture of a medicament for activing cAMP signaling and simultaneously blocking β-arrestin recruitment in vitro or in vivo.

In one embodiment, the melanocortin receptor or the melanocortin receptor homodimer is MC1R, MC3R, MC4R or MC5R.

In one embodiment, the melanocortin receptor or the melanocortin receptor homodimer is MC3R.

Another embodiment of the invention provides a method of modulating (e.g., increasing or decreasing) metabolic activity in an animal in need thereof, comprising administering an effective amount of a compound of formula I, or a pharmaceutically acceptable salt thereof, to the animal.

Another embodiment of the invention provides a compound of formula I, or a pharmaceutically acceptable salt thereof for use in modulating (e.g., increasing or decreasing) metabolic activity.

Another embodiment of the invention provides the use of a compound of formula I, or a pharmaceutically acceptable salt thereof for the manufacture of a medicament for modulating (e.g., increasing or decreasing) metabolic activity in an animal in need thereof.

Another embodiment of the invention provides a method of modulating (e.g., increasing or decreasing) appetite in an animal in need thereof, comprising administering an effective amount of a compound of formula I, or a pharmaceutically acceptable salt thereof, to the animal.

Another embodiment of the invention provides a compound of formula I, or a pharmaceutically acceptable salt thereof for use in modulating (e.g., increasing or decreasing) appetite.

Another embodiment of the invention provides the use of a compound of formula I, or a pharmaceutically acceptable salt thereof for the manufacture of a medicament for modulating (e.g., increasing or decreasing) appetite in an animal in need thereof.

Another embodiment of the invention provides a method of decreasing food intake, reducing body fat percentage, and/or increasing fat consumption in an animal in need thereof, comprising administering an effective amount of compound of formula I, or a pharmaceutically acceptable salt thereof, to the animal.

Another embodiment of the invention provides a compound of formula I, or a pharmaceutically acceptable salt thereof, for use in decreasing food intake, reducing body fat percentage, and/or increasing fat consumption in an animal in need thereof.

Another embodiment of the invention provides the use of a compound of formula I, or a pharmaceutically acceptable salt thereof, for the manufacture of a medicament for decreasing food intake, reducing body fat percentage, and/or increasing fat consumption in an animal in need thereof.

Another embodiment of the invention provides a method of activating one downstream signaling event and simultaneously blocking a different downstream signaling event of a G protein-couple receptor (GPCR) homodimer comprising contacting the GPCR homodimer a ligand that comprises an agonist pharmacophore and an antagonist pharmacophore, wherein the agonist pharmacophore occupies and activates one receptor within the GPCR homodimer and the antagonist pharmacophore occupies and deactivates the other receptor within the GPCR homodimer.

In one embodiment, the GPCR homodimer is a melanocortin receptor homodimer.

In one embodiment, the agonist pharmacophore activates cAMP signaling and the antagonist pharmacophore deactivates β-arrestin recruitment.

In one embodiment, the agonist pharmacophore is linked to the antagonist pharmacophore through a linking group.

In one embodiment, the agonist pharmacophore comprises an amino acid sequence of SEQ ID NO:1.

In one embodiment, the antagonist pharmacophore comprises an amino acid sequence of SEQ ID NO:2.

Compounds of the invention can also be administered in combination with other therapeutic agents, for example, other agents that are useful for the obesity. Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to treat obesity.

In cases where compounds are sufficiently basic or acidic, a salt of a compound of formula (I) can be useful as an intermediate for isolating or purifying a compound of formula (I). Additionally, administration of a compound of formula (I) as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Compounds of formula (I) (including salts and prodrugs thereof) can be formulated as pharmaceutical compositions and administered to a mammalian host, such as a human patient in a variety of forms adapted to the chosen route of administration, i.e., orally or parenterally, by intravenous, intramuscular, topical, nasal, inhalation, suppository, sub dermal osmotic pump, or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula Ito the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compound of formula (I) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents. For example, compounds of formula (I), or salts thereof, may be administered with other agents that are useful for modulating appetite (i.e., increasing or decreasing), modulating metabolic activity, treating obesity or diseases associated with obesity (e.g., diabetes, cardiovascular disease or hypertension), inducing weight loss, increasing or decreasing weight gain. Accordingly, in one embodiment the invention also provides a composition comprising a compound of formula (I), or a pharmaceutically acceptable salt thereof, at least one other therapeutic agent, and a pharmaceutically acceptable diluent or carrier. The invention also provides a kit comprising compound of formula (I), or a pharmaceutically acceptable salt thereof, at least one other therapeutic agent, packaging material, and instructions for administering the compound of formula (I) or the pharmaceutically acceptable salt thereof and the other therapeutic agent or agents to an animal to modulate appetite, modulate metabolic activity, treat obesity or diseases associated with obesity (e.g., diabetes, cardiovascular disease or hypertension), induce weight loss, increase weight gain, or decrease weight gain.

The invention will now be illustrated by the following non-limiting Examples.

### EXAMPLE 1

### Design and Synthesis of Ligands

Homobivalent ligands targeting melanocortin receptors have previously resulted in increased binding affinity (˜14 to 25-fold) consistent with a synergistic binding mode resulting from receptor dimer binding (Fernandes, S. M. et al. Bioorg. Med. Chem. 2014, 22, 6360-6365; Lensing, C. J. et al. J. Med. Chem. 2016, 59, 3112-3128; Lensing, C. J. et al. ACS Chem. Neurosci. 2017, 8, 1262-1278; Brabez, N. et al. J. Med. Chem. 2011, 54, 7375-7384; Carrithers, M. D. et al. Chem. Biol. 1996, 3, 537-542; Elshan, N. G. R. D. et al. Org. Biomol. Chem. 2015, 13, 1778-1791; Handl, H. L. et al. Bioconjugate Chem. 2007, 18, 1101-1109; Vagner, J. et al. Bioorg. Med. Chem. Lett. 2004, 14, 211-215; Bowen, M. E. et al. J. Org. Chem. 2007, 72, 1675-1680; Jagadish, B. et al. Bioorg. Med. Chem. Lett. 2007, 17, 3310-3313; Dehigaspitiya, D. C. et al. Tetrahedron Lett. 2015, 56, 3060-3065 and Dehigaspitiya, D. C. et al. Org. Biomol. Chem. 2015, 13, 11507-11517). In spite of an increased binding affinity, much smaller fold increases in cAMP based functional activity have been observed (3 to 5-fold) (Lensing, C. J. et al. J. Med. Chem. 2016, 59, 3112-3128). Hruby and coworkers noted similar effects with melanocortin bivalent ligands in which cAMP accumulation was not as dramatically increased with synergistic multivalent binding (Brabez, N. et al. J. Med. Chem. 2011, 54, 7375-7384). One possibility for the incongruity between binding affinity increases and functional signaling increases with bivalent ligands may be due to allosterism between the melanocortin receptors within homodimers (Lensing, C. J. et al. J. Med. Chem. 2016, 59, 3112-3128). Such asymmetric signaling with GPCR homodimers has previously been reported for a variety of systems including the vasopressin (Orcel, H. et al. Mol. Pharmacol. 2009, 75, 637-647), dopamine (Han, Y. et al. Nat. Chem. Biol. 2009, 5, 688-695), adenosine (Gracia, E. et al. Neuropharmacology 2013, 71, 56-69), metabotropic glutamate (Kniazeff, J. et al. Nat. Struct. Mol. Biol. 2004, 11, 706-713), and serotonin receptors (Pellissier, L. P. et al. J. Biol. Chem. 2011, 286, 9985-9997).

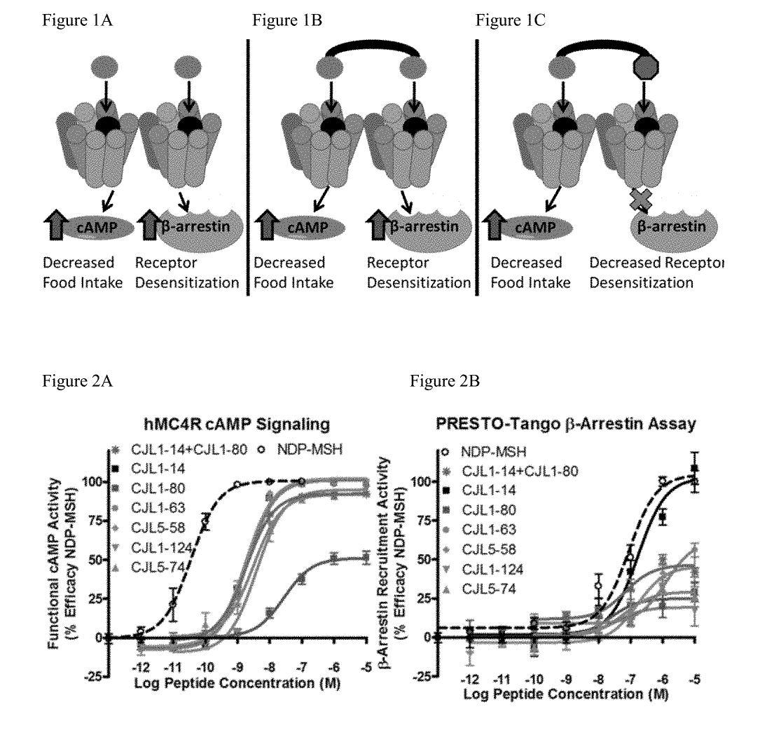

A new paradigm can be hypothesized in which one receptor within the melanocortin homodimer might be responsible for cAMP signaling and the other receptor might be responsible for signaling through a different cellular pathway (e.g. β-arrestin recruitment pathway) (FIGS. 1A-1B). It would then follow that the increased binding would not necessarily result in an increase in functional agonist activity observed in a cAMP assay, since the effect of the second binding event is not detected by this cellular assay paradigm. In order to the exploit this possibility of asymmetric homodimers, synthesized MUmBLs (melanocortin unmatched bivalent ligands) that contained the known agonist melanocortin moiety His-DPhe-Arg-Trp on one side of the molecule (Haskell-Luevano, C. et al. J. Med. Chem. 1997, 40, 2133-2139 and Haskell-Luevano, C. et al. J. Med. Chem. 2001, 44, 2247-2252), and the known MC3R and MC4R antagonist moiety His-DNa!(2′)-Arg-Trp (Holder, J. R. et al. J. Med. Chem. 2002, 45, 3073-3081 and Chen, M. et al. Peptides 2006, 27, 2836-2845) connected by three different previously validated linker systems (Table 1), were designed (Fernandes, S. M. et al. Bioorg. Med. Chem. 2014, 22, 6360-6365; Lensing, C. J. et al. J. Med. Chem. 2016, 59, 3112-3128; Handl, H. L. et al. Bioconjugate Chem. 2007, 18, 1101-1109 and Josan, J. S. et al. Int. J. Pept. Res. Ther. 2008, 14, 293-300).

Because bivalent ligands presumably occupy both sites in a receptor dimer due to synergistic binding, a MUmBL is postulated to occupy one receptor within a dimer pair with an agonist pharmacophore and the other receptor within the same dimer with an antagonist pharmacophore (FIG. 1C). This assumes approximately equal binding affinities of the pharmacophores, and low enough concentrations of ligand so that intermolecular competition does not occur. The MUmBLs should favor a bivalent binding mode over a monovalent binding mode (due to increased binding affinity to dimers supported previously in competitive binding experiments) (Lensing, C. J. et al. J. Med. Chem. 2016, 59, 3112-3128). This should shift the equilibrium towards occupation of one receptor with agonist scaffold and the other receptor with antagonist scaffold in the homodimer, but other binding states probably exist in equilibrium.

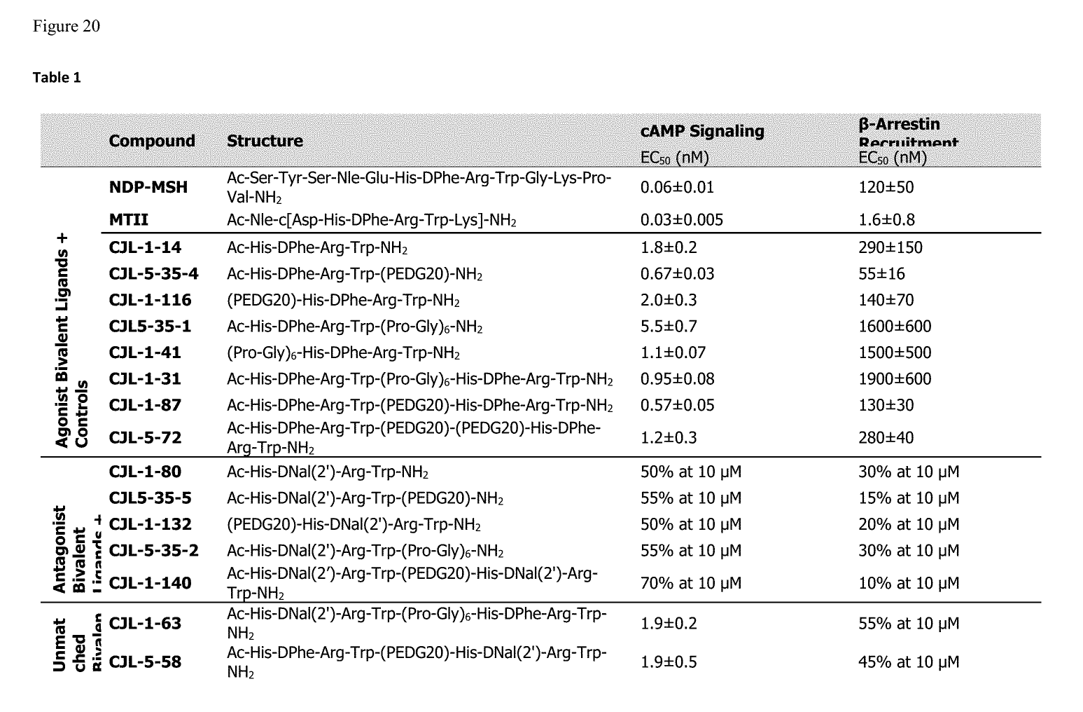

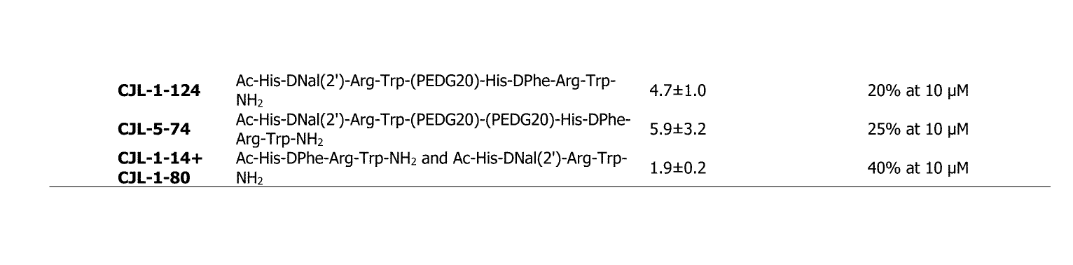

Ligands CJL-1-124, CJL-5-74, and CJL-1-63 feature the His-DPhe-Arg-Trp scaffold on the C-terminus and the His-DNa!(2′)-Arg-Trp scaffold on the N-terminus (FIG. 20, Table 1). Since the molecules are not symmetric, the opposite composition of CJL-1-124 was designed and synthesized with the His-DNa!(2′)-Arg-Trp scaffold on the C-terminus and His-DPhe-Arg-Trp scaffold on the N-terminus in compound CJL-5-58. In particular, the construction of CJL-5-58 with the PEDG20 linker system was selected because this linker system was previously shown to be optimal in homobivalent ligands compared to the PEDG20-PEDG20 or Pro-Gly linker systems at the mMC4R (Fernandes, S. M. et al. Bioorg. Med. Chem. 2014, 22, 6360-6365 and Lensing, C. J. et al. J. Med. Chem. 2016, 59, 3112-3128).

### Chemical Synthesis

Peptides were synthesized utilizing standard solid phase peptide synthesis and fluorenyl-9-methoxycarbonyl (Fmoc) methodologies to protect the elongating peptide chain (Stewart, J. M. et al. Solid Phase Peptide Synthesis. 2^{nd} edition (Pierce Chemical Co., 1984) and Carpino, L. A. et al. J. Org. Chem. 1972, 37, 3404-3409). A CEM Discover SPS microwave peptide synthesizer was used to expedite couplings and deprotections. A split resin technique was used to synthesize common sequences as previously described (Lensing, C. J. et al. J. Med. Chem. 2016, 59, 3112-3128). The O-(N-Fmoc-3-aminopropyl)-O′-(N-diglycolyl-3-aminopropyl)-diethyleneglycol [Fmoc-NH-(PEG)₂-COOH (20atoms) or Fmoc-NH₂-PEDG20-COOH] was purchased from Novobiochem® EMD Millipore Corp (Billerica, Mass., USA). The N,N-diisopropylethylamine (DIEA), triisopropylsilane (TIS), 1,2-ethanedithiol (EDT), piperidine, pyridine, and trifluoroacetic acid (TFA) were purchased from Sigma-Aldrich (St. Louis, Mo.). The 4-(2′,4′-dimethoxyphenyl-Fmoc-aminomethyl)phenoxyacetyl-MBHA resin [Rink-amide-MBHA (200-400 mesh), 0.35-0.37 meq/g substitution], 2-(1H-benzotriazol-1-yl)-1,1,3,3-tetramethyluronium hexafluorophosphate (HBTU), and Fmoc-protected amino acids [Fmoc-Pro, Fmoc-Gly, Fmoc-His(Trt), Fmoc-DPhe, Fmoc-Arg(Pbf), Fmoc-Trp(Boc), and Fmoc-DNal(2′)] were purchased from Peptides International (Louisville, Ky., USA). Acetonitrile (MeCN), N,N-dimethylformamide (DMF), acetic anhydride, dichloromethane (DCM), and methanol (MeOH) were purchased from FischerScientific. All reagents were ACS grade or better and were used without further purification.

Peptides were assembled in a fritted polypropylene reaction vessel (25 mL CEM reaction vessel) on the Rink-amide-MBHA resin. A repeated two-step cycle of deprotection with 20% piperidine in DMF, then amide coupling with the Fmoc-amino acid, HBTU, and DIEA was employed until the final peptide was synthesized on resin. Excess reagents were removed between all deprotection or coupling by 3-5 washes of DMF between steps. A Kaiser/ninhydrin test was used after each deprotection or coupling step (except with Pro residues) to indicated the presence or lack of a free primary amine (Kaiser, E. et al. Anal. Biochem. 1970, 34, 595-598). For Pro residues, the presence or lack of a free secondary amine was indicated by a chloranil test (Stewart, J. M. et al. Solid Phase Peptide Synthesis. 2^{nd} edn, (Pierce Chemical Co., 1984) and Christensen, T. Acta Chem. Scand. 1979, 33, 763-766). Removal of the Fmoc group was achieved in a twostep process. First an initial two minute deprotection was performed outside of the microwave. Then a second aliquot of 20% piperidine was added and further deprotection was assisted by microwave heating (75° C., 30 W, 4 min).

Amide coupling was achieved by addition of 3.1-fold excess Fmoc-protected amino acids (5.1-fold for Arg) and 3-fold excess of HBTU (5-fold for Arg) in DMF added to the free amine on the elongating peptide on the resin. After which the 5-fold excess of DIEA (7-fold for Arg) was added, and the reaction was heated in the microwave synthesizer (75° C., 30 W or 50° C., 30 W for His) for five minutes (10 min for Arg). The Fmoc-NH-(PEDG20)-COOH was incorporated into the peptide using the same protocol except it was allowed to cool for at least one hour after microwave heating to ensure the reaction went to completion.

Acetylation was achieved on resin after the final Fmoc deprotection by addition of 3:1 mixture of acetic anhydride to pyridine and were mixed at room temperature with bubbling nitrogen for 30 minutes. Before cleavage, all peptides were washed with DCM at least 3 times and dried in a desiccator. Side chain deprotection and resin cleavage was simultaneously accomplished via addition of 8 mL of a cleavage cocktail (91% TFA, 3% EDT, 3% TIS, 3% water) for 1.5-3 hours. Peptides were precipitated from cleavage solution using cold (4° C.) anhydrous diethyl ether. The cloudy mixture of peptides was vortexed and centrifuged at 4° C. and 4000 RPMs for 4 minutes (Sorval Super T21 high-speed centrifuge swinging bucket rotor). The supernatant was discarded. The crude peptide pellet was then washed with cold (4° C.) diethyl ether and centrifuged. This process was repeated until no thiol aroma was present (usually 3 times) and the peptides were dried overnight in a desiccator.

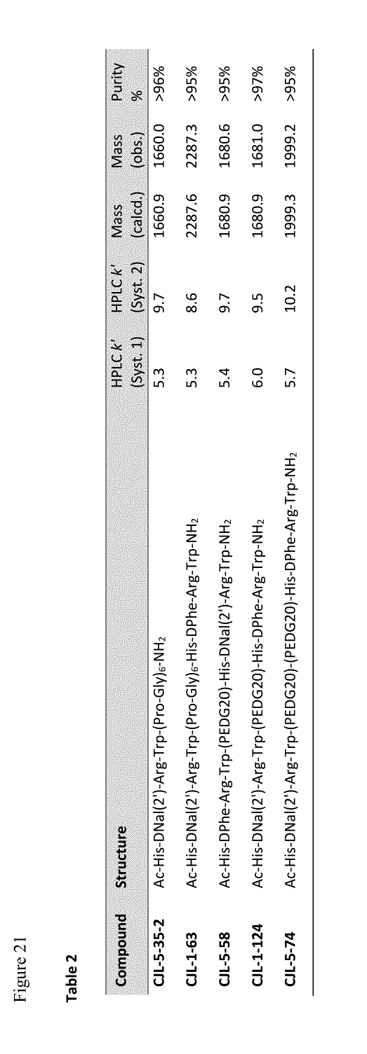

A Shimadzu chromatography system with a photodiode array detector and a semipreparative RP-HPLC C₁₈ bonded silica column (Vydac 218TP1010, 1 cm×25 cm) were used to purify 5-20 mg sample of crude peptide by RP-HPLC. The solvent system for purification was either MeCN or MeOH in 0.1% aqueous TFA. Purified fractions were collected and peptides were concentrated in vacuo and lyophilized. A purity of 95% or greater was confirmed by RP-HPLC in two diverse solvent systems (10% MeCN in 0.1% TFA/water and a gradient to 90% MeCN over 35 min; and 10% MeOH in 0.1% TFA/water and a gradient to 90% MeOH over 35 minutes). ESI-MS was used to confirm the correct molecular mass (University of Minnesota Department of Chemistry Mass Spectrometry Laboratory) (FIG. 21, Table 2).

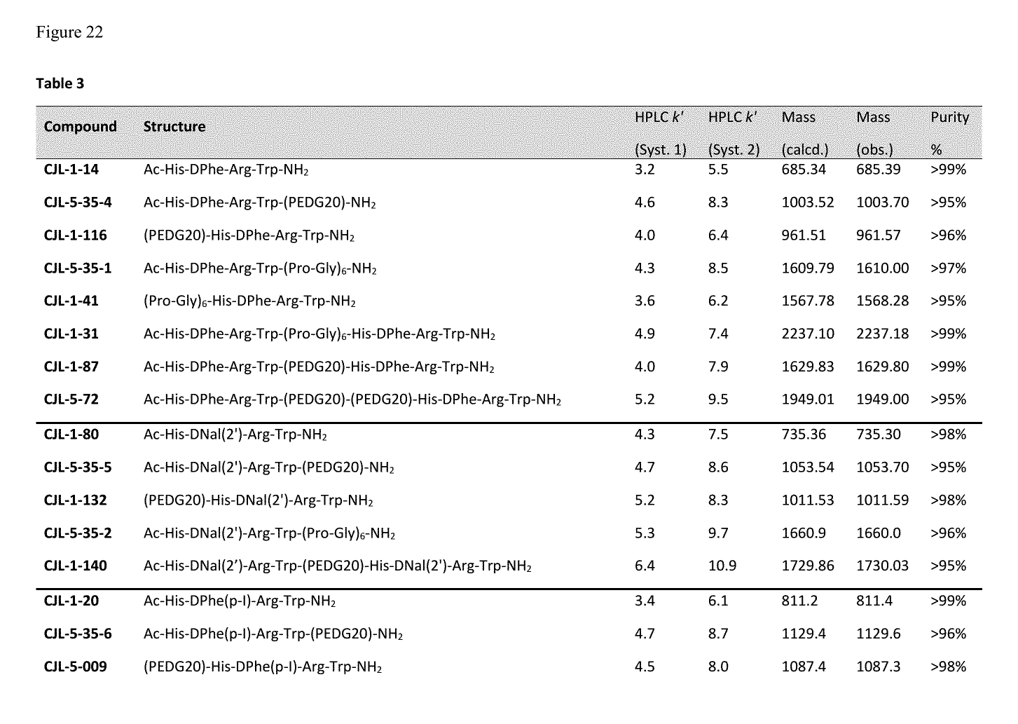

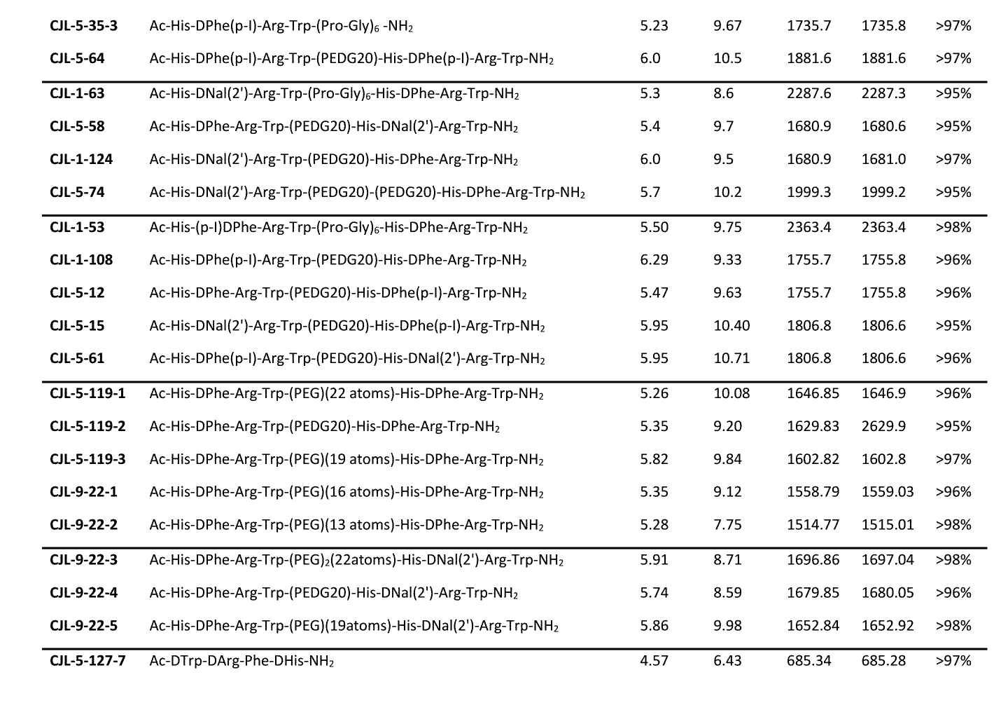

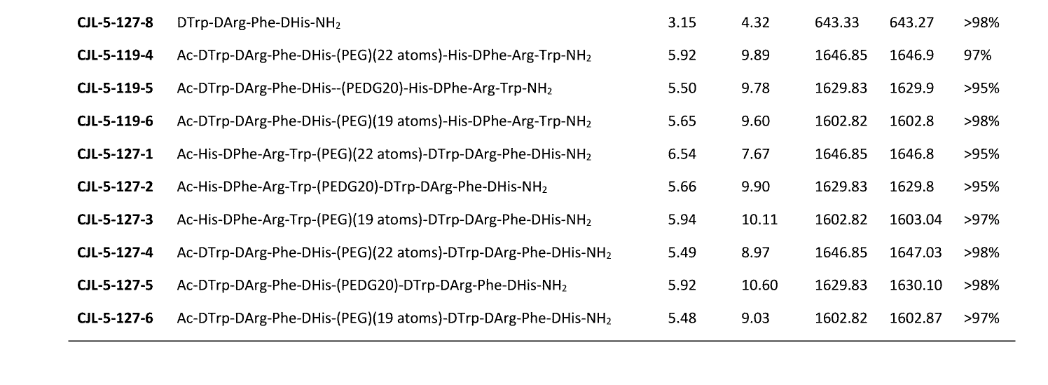

Additional ligands were synthesized and purified in an analogous fashion (FIG. 22, Table 3).

### EXAMPLE 2

### Biased Signaling at the hMC4R

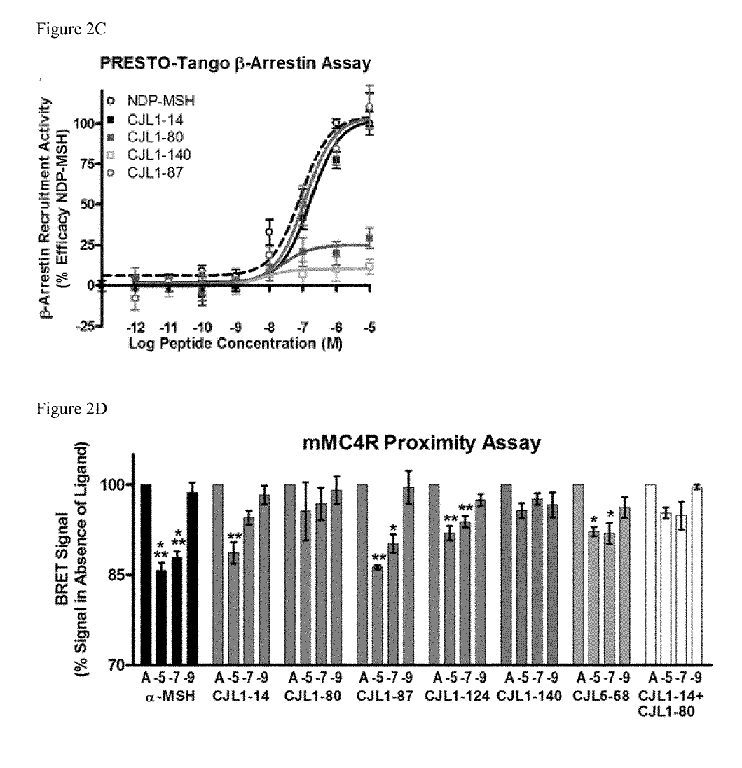

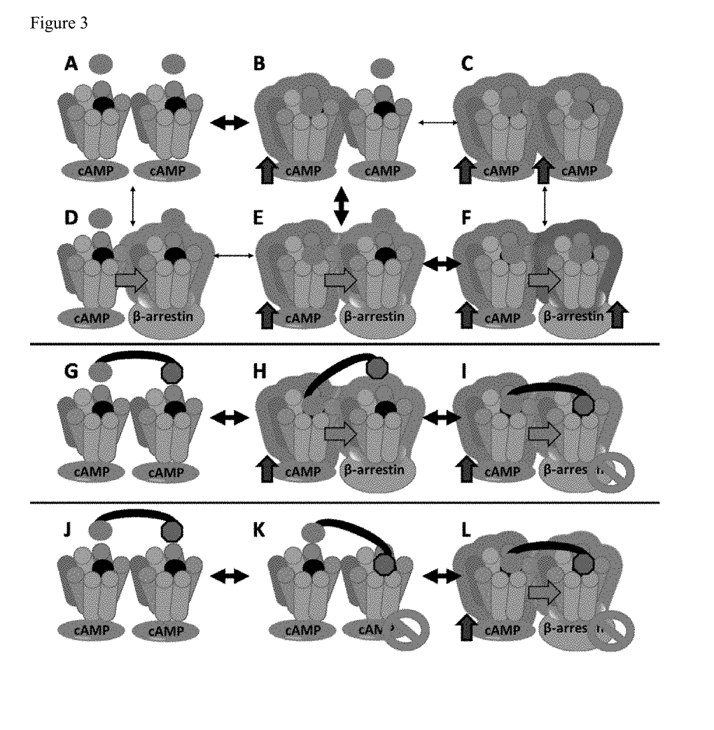

Upon agonist stimulation, melanocortin receptors are known to signal through a Gαₛ-protein mediated signaling pathway that results in intracellular cAMP accumulation. Agonist stimulation of the melanocortin receptors also results in β-arrestin recruitment and receptor desensitization (Shinyama, H. et al. Endocrinology 2003, 144, 1301-1314; Cai, M. Y. et al. Chem. Biol. Drug Des. 2006, 68, 183-193 and Gao, Z. H. et al. J. Pharmacol. Exp. Ther. 2003, 307, 870-877). In order to evaluate the ligands efficacy and potency to stimulate cAMP signaling, ALPHAScreen™ cAMP Assay Technology was utilized to assess live HEK293 cells stably expressing human (h)MC4R (Xiang, Z. et al. Biochemistry 2010, 49, 4583-4600 and Xiang, Z. et al. Biochemistry 2006, 45, 7277-7288). All ligands that contained the His-DPhe-Arg-Trp pharmacophore, including the MUmBLs, were single-digit or sub-nanomolar agonists in the cAMP assay (FIG. 20, Table 1 and FIG. 2A). The most potent ligand (besides control ligand NDP-MSH) was the bivalent ligand CJL-1-87 that had an EC₅₀ of 570 pM and was 3-fold more potent than its monovalent counterpart CJL-1-14. This result was similar to that previously observed with CJL-1-87 at the mouse (m)MC4R (Lensing, C. J. et al. J. Med. Chem. 2016, 59, 3112-3128). The ligands that only contained the His-DNal(2′)-Arg-Trp antagonist scaffold were not able to elicit a full response when tested up to 10 μM. Homobivalent ligand CJL-1-140 with two His-DNal(2′)-Arg-Trp resulted in 70% cAMP accumulation of that seen with NDP-MSH at 10 μM which is also consistent with previous reports at the mouse receptors (Lensing, C. J. et al. J. Med. Chem. 2016, 59, 3112-3128). These results suggest that there is minimal species variation within the monovalent and homobivalent ligands currently tested.

The MUmBLs (i.e. CJL-1-63, CJL-5-58, CJL-1-124, and CJL-5-74) were all single digit nanomolar agonists at the hMC4R. For comparison with the MUmBLs and as a control, an equal mixture of tetrapeptides CJL-1-14+CJL-1-80 was assayed. In order to give the best comparison to the MUmBLs, 1 nM of the tetrapeptide mixture contained 1 nM CJL-1-14 and 1 nM CJL-1-80 (for a final concentration of 2 nM total peptide) was tested. This would be directly comparable to 1 nM of a MUmBL when looking at final pharmacophore concertation. This tetrapeptide mixture resulted in an agonist dose response curve with an EC₅₀ of 1.9±0.2 nM. From this data, it appears that antagonist scaffold His-DNal(2′)-Arg-Trp is not capable of effecting the cAMP agonist pharmacology of His-DPhe-Arg-Trp agonist scaffold when mixed in equal portions.

Theoretically, if both the agonist scaffold and antagonist scaffold compete equally for binding, then at 100% receptor occupancy 50% of the receptors would be occupied by agonist tetrapeptide scaffold and 50% would be occupied by the antagonist tetrapeptide scaffold. This likelihood of 50:50 binding should be amplified by the synergistic bivalent binding mode (Lensing, C. J. et al. J. Med. Chem. 2016, 59, 3112-3128). Based on this assumption of 50:50 binding, the MUmBLs full cAMP agonist pharmacology would be achieved by only 50% receptor occupancy by the agonist scaffold at the receptors, since the antagonist scaffold would be occupying approximately 50% of the receptors. This is consistent with both the spare receptor theory (Stephenson, R. P. Br. J. Pharmacol. Chemother. 1956, 11, 379-393 and Takeyasu, K. et al. Life Sci. 1979, 25, 1761-1771), and the hypothesis presented above for asymmetric signaling homodimers in which ˜50% of the receptors are responsible for β-arrestin recruitment and 50% are responsible for cAMP signaling (FIGS. 1A-1C). In practice, the MUmBLs may be binding to the melanocortin receptor monomers, dimers, and/or higher-order oligomers and may not be binding in exactly equal amounts of agonist scaffold and antagonist scaffold due to intermolecular competition. However, the synergistic binding previously achieved would only be observed if bivalent ligands are binding at a ratio of one MUmBL per dimer (two receptors) and the equilibrium should be favor this bivalent binding mode.

It was, therefore, hypothesized that the second binding event within the GPCR dimer may be responsible for a different functional response not detected in the cAMP functional assays. It has previously been observed that β-arrestin recruitment of one protomer within the AT₁ angiotensin receptor homodimer can be allosterically regulated by selective stimulation of the other protomer (Szalai, B. et al. Biochem. Pharmacol. 2012, 84, 477-485). In order to examine if β-arrestin recruitment to the hMC4R was regulated differently by MUmBLs versus agonist or antagonist homobivalent ligands, we utilized the PRESTO-Tango assay developed by Roth and colleagues (Kroeze, W. K. et al. Nat. Struct. Mol. Biol. 2015, 22, 362-369 and Barnea, G. et al. Proc. Natl. Acad. Sci. U. S. A. 2008, 105, 64-69). The PRESTO-Tango technology is an open-source resource that has been utilized to identify ligands for orphan receptors based on β-arrestin recruitment. This assay has previously been validated at the hMC4R that agonist stimulation results in β-arrestin recruitment (Kroeze, W. K. et al. Nat. Struct. Mol. Biol. 2015, 22, 362-369). In agreement with these results, classic monovalent agonist ligands result in the recruitment of β-arrestin and high signal (FIG. 20, Table 1 and FIGS. 2B-2C). The classical melanocortin control agonists NDP-MSH, MTII and the tetrapeptide Ac-His-DPhe-Arg-Trp-NH₂ all resulted in maximal β-arrestin recruitment with MTII being the most potent ligand. The linker control and homobivalent ligands that featured only the His-DPhe-Arg-Trp pharmacophore all resulted in maximal β-arrestin recruitment relative to NDP-MSH control. Among the linker controls, compound CJL-5-35-4 with the PEDG20 linker on the C-terminus resulted in a 5-fold increase in β-arrestin recruitment compared to the tetrapeptide CJL-1-14. This ligand also resulted in a 3-fold increase in the cAMP signaling assay. The other PEDG20 linker compound CJL-1-116 resulted in less than a 3-fold increase in β-arrestin recruitment compared to CJL-1-14. The His-DPhe-Arg-Trp that utilized the Pro-Gly linker system did result in a decrease in the potency for β-arrestin recruitment in spite them retaining their full cAMP pathway functional activity.

The ligands containing only the antagonist His-DNal(2′)-Arg-Trp pharmacophore resulted in minimal β-arrestin recruitment consistent with a classical antagonist pharmacology. The tetrapeptide Ac-His-DNal(2′)-Arg-Trp-NH₂ resulted in 30% response at 10 μM compared to the maximal efficacy of NDP-MSH. The other linker control ligands and the bivalent ligand CJL-1-140 resulted in equal or lower β-arrestin recruitment. This result was not surprising given the antagonist nature of these compounds and that antagonist have previously been reported to result in minimal β-arrestin recruitment and receptor internalization (Shinyama, H. et al. Endocrinology 2003, 144, 1301-1314; Cai, M. Y. et al. Chem. Biol. Drug Des. 2006, 68, 183-193 and Gao, Z. H. et al. J. Pharmacol. Exp. Ther. 2003, 307, 870-877).

The MUmBLs resulted in minimal β-arrestin recruitment. The most potent MUmBL was CJL-1-63 that resulted in 55% maximal efficacy at 10 μM compared to NDP-MSH. All other MUmBLs resulted in less β-arrestin recruitment. Because these ligands still potently stimulate cAMP signaling but result in minimal β-arrestin recruitment, it supports the current hypothesis that one pharmacophore is responsible for the activation of the cAMP pathway, but the other pharmacophore is responsible for the β-arrestin recruitment. When a bivalent ligand is comprised of an agonist scaffold and an antagonist scaffold, it should favor a binding mode in which equal portions of agonist scaffold and antagonist scaffold bind to a GPCR dimer as discussed above (i.e. one MUmBL to two receptors or one dimer). The agonist pharmacophore would then signal effectively through the cAMP pathway, but the antagonist pharmacophore would block the β-arrestin recruitment pathway (FIGS. 1A-1C). The current data observed with MUmBLs are not consistent with the current dogma in the field, however, these data may be explained by the asymmetric allosteric signaling within melanocortin homodimers.

An explanation of the biased agonism is through a model for allosterically interacting receptor dimers (FIG. 3) (Durroux, T. Trends Pharmacol. Sci. 2005, 26, 376-384 and Casado, V. et al. Pharmacol. Ther. 2007, 116, 343-354). In this model, one receptor within a dimer can allosterically stabilize the other receptor within the dimer to different conformations. These different conformations are thought to be dynamic in that the receptors oscillate between the different states even with no ligand present (Durroux, T. Trends Pharmacol. Sci. 2005, 26, 376-384). However, it is postulated that with no ligand bound both receptors are conformationally open to cAMP signaling upon agonist stimulation (FIG. 3, state A). After the first agonist binding event, a conformational change occurs which induces cAMP signaling pathway (FIG. 3, state B) and this conformational change allosterically modifies the second receptor to have a propensity to signal through the β-arrestin pathway (FIG. 3, state E). For this reason, monovalent agonist ligands, homobivalent agonist ligands, and the MUmBLs all produce full agonist cAMP induction, since the first agonist binding event is similar. After the first agonist binding event, the second receptor in the dimer is hypothesized to have structural bias for β-arrestin recruitment upon agonist binding. Therefore, the second agonist binding event results in β-arrestin recruitment (FIG. 3, state F). This is the same for monovalent and homobivalent agonist ligands since both result in a second agonist binding event and this is observed in full β-arrestin recruitment results (FIGS. 2B-2C). However, the MUmBLs result in an antagonist binding the second receptor instead of another agonist. The MUmBL's antagonist tetrapeptide scaffold prevents β-arrestin recruitment that results in minimal signal in the PRESTO-Tango assay (FIG. 3, states C-I).

There is an assumption above that the agonist tetrapeptide scaffold of the MUmBLs binds first before the antagonist tetrapeptide scaffold, but in practice the order of binding is not determined (FIG. 3, states J-L). However, antagonist scaffolds restrict the GPCR from accessing conformational states that result in GPCR signaling (i.e. G-protein or β-arrestin). Therefore, even if the antagonist does bind first to the receptor dimer pair (FIG. 3, state K), it is would not induce G-protein signaling. Instead it would still require the first agonist binding event to the second receptor in the dimer pair to occur that would result in cAMP signaling (FIG. 2A), and allosteric modulation to the β-arrestin ready state. However, the antagonist scaffold would already be bond to the dimer, and would block β-arrestin recruitment resulting in minimal PRESTO-Tango signal (FIG. 2B, FIG. 3, state L).

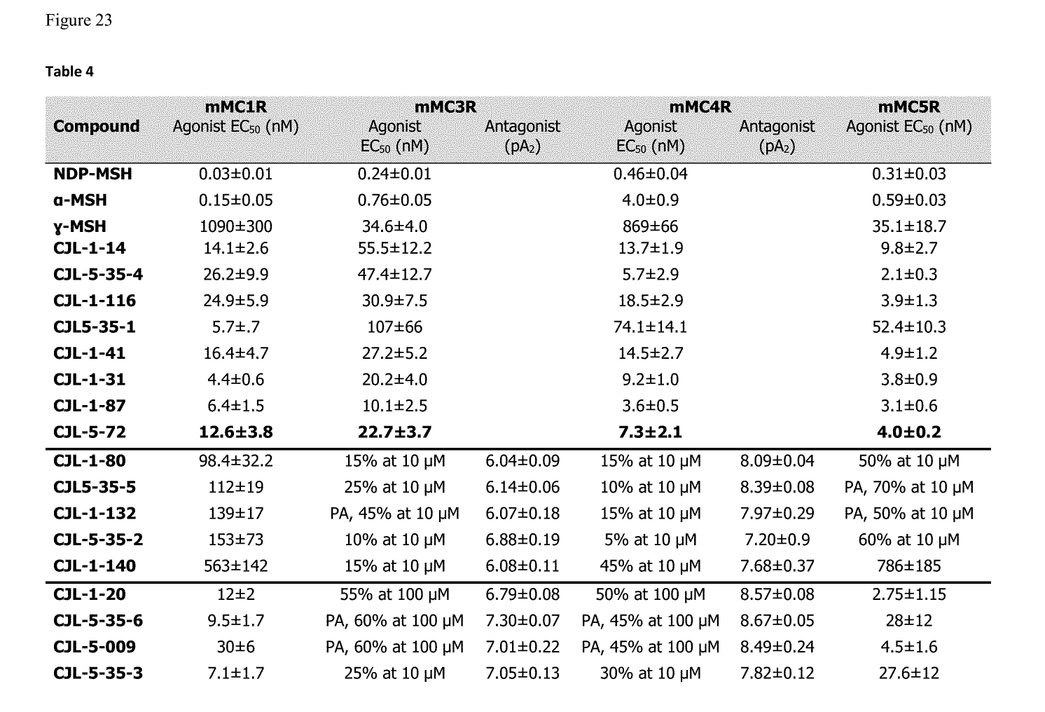

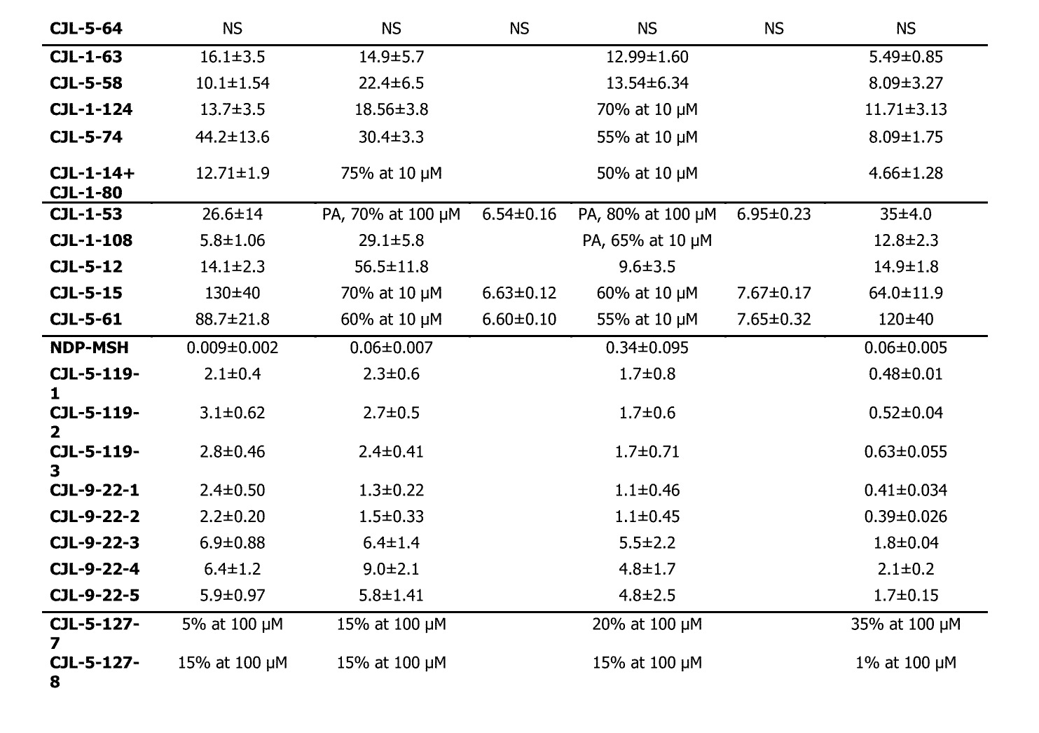

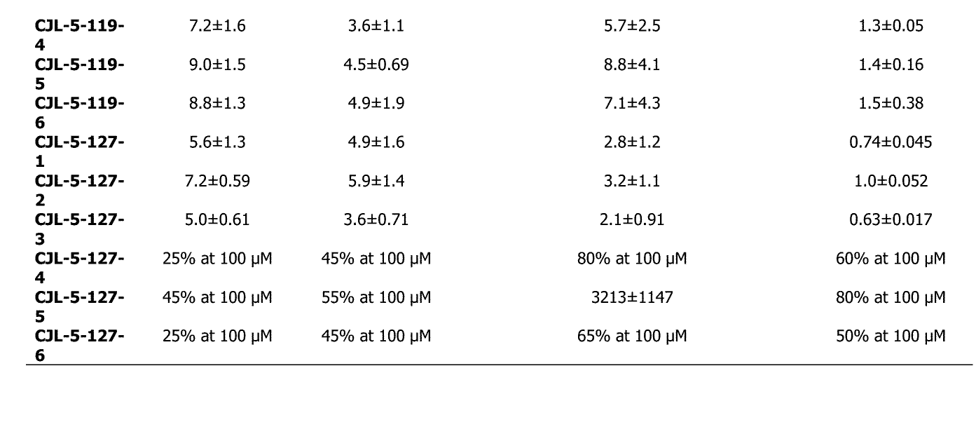

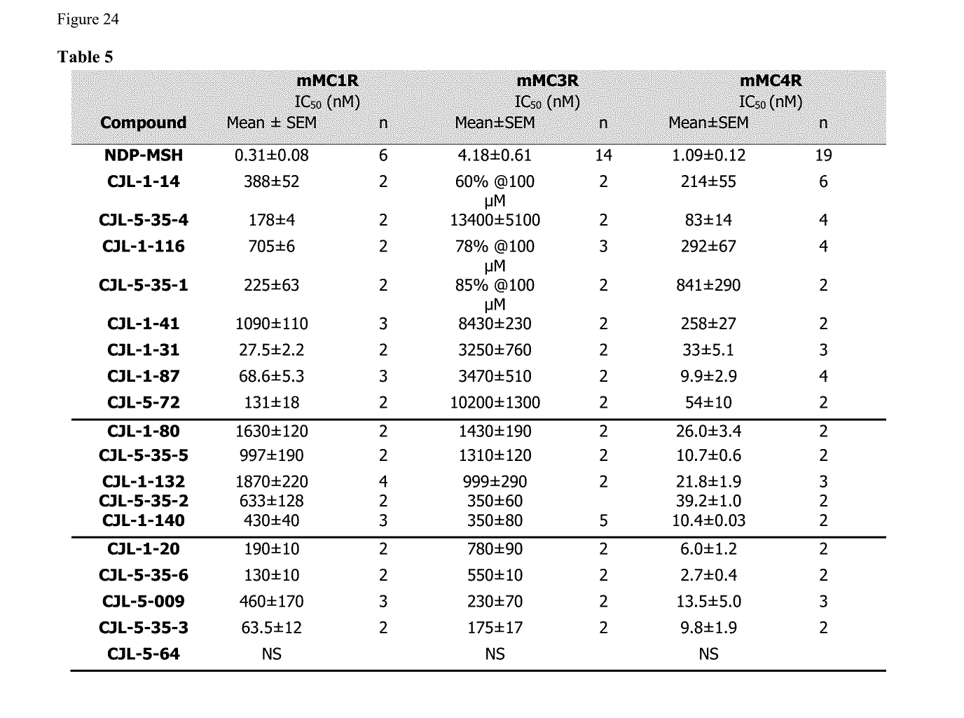

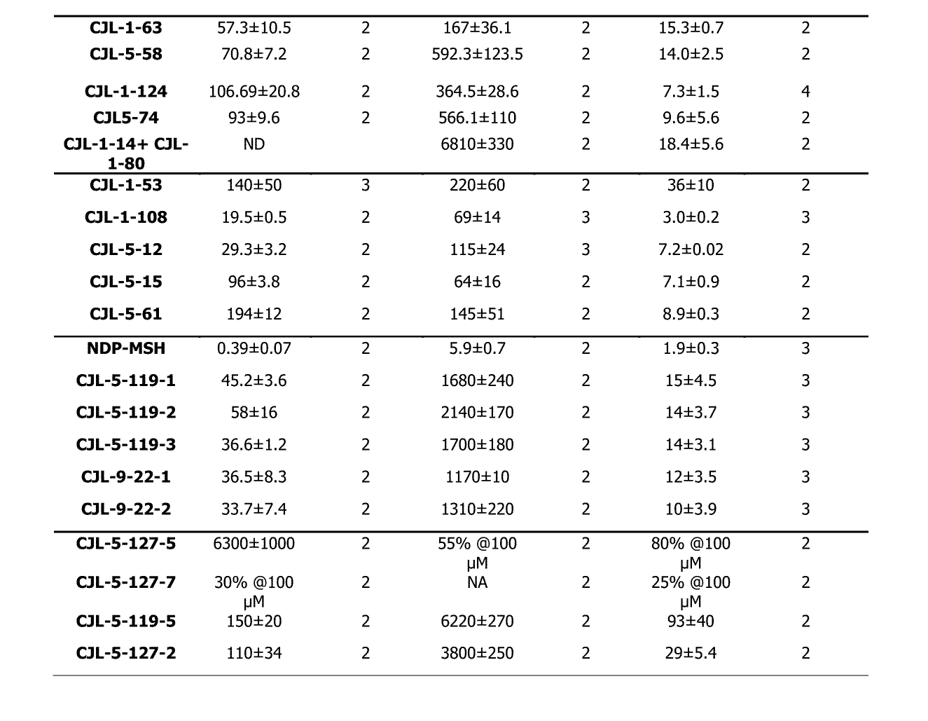

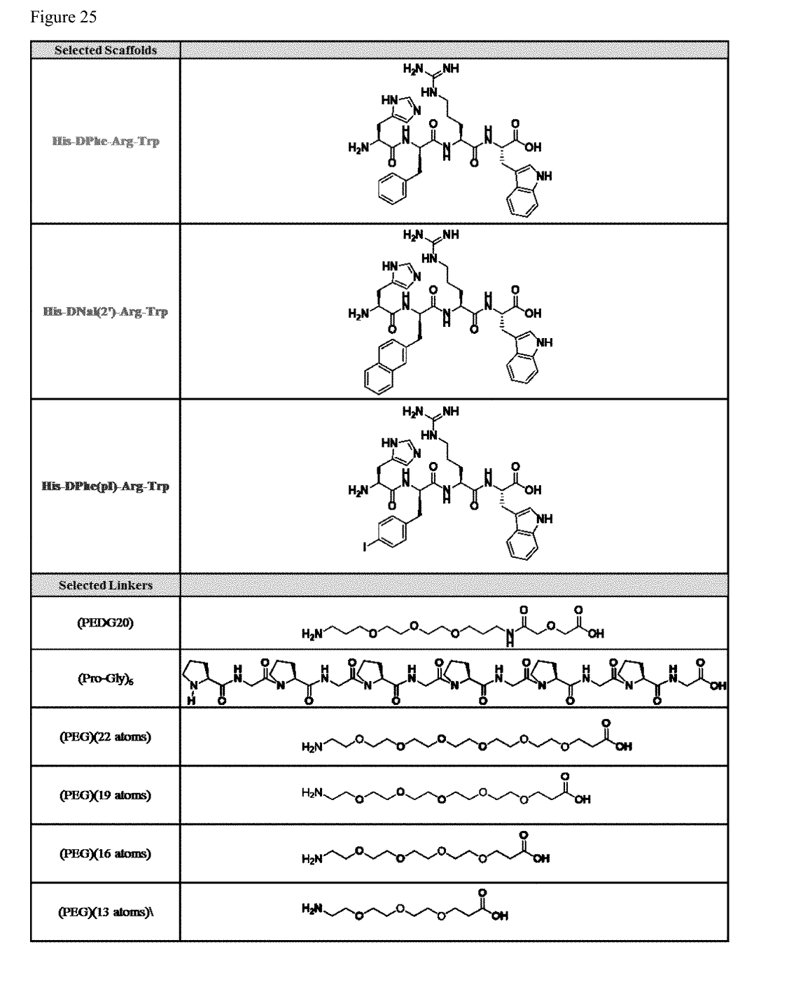

Functional activity data and competitive binding data of the ligands at the mouse melanocortin receptor subtypes are summarized in FIG. 23, Table 4 and FIG. 24, Table 5.

### Cell Culture

HEK293 cells for the ALPHAScreen assay, competitive binding assay, and PRESTO-Tango assay were maintained in humidified atmosphere of 95% air and 5% CO₂ at 37° C. in Dulbecco's modified Eagle's medium (DMEM) supplemented with 10% newborn calf serum (NCS), and 1% penicillin/streptomycin. Stable cell lines were generated with wild type mMC1R, mMC4R, mMC5R, hMC4R-Flag, and mMC3R-Flag DNA in pCDNA₃ expression vector (20 μg) using the calcium phosphate transfection method (Chen, C. A. et al. Biotechniques 1988, 6, 632-638). Stable populations were selected for using G418 selection (0.7-1.0 mg/mL) and used in bioassays unless indicated otherwise. In vitro experimental ligands were dissolved to a 10⁻² M stock in DMSO and stored at −20° C. Subsequent dilutions were performed in each assay's specific buffer to achieve the final concentration in the well. The ligands were assayed as TFA salts.

AlphaScreen cAMP Functional Bioassay

The AlphaScreen® cAMP technology (PerkinElmer Life Sciences, Cat #6760625M) was utilized to measure cAMP signaling after ligand stimulation in HEK293 cells stably expressing the mMC1R, mMC3R, mMC4R, hMC4R and mMC5R. The AlphaScreen® assay was performed as described by manufacturer. This method has been previously utilized by our lab (Lensing, C. J. et al. J. Med. Chem. 2016, 59, 3112-3128 and Singh, A. et al. ACS Med. Chem. Lett. 2015, 6, 568-572), and it is described briefly below.

On the day of the assay, cells were 70-95% confluent in 10 cm plates. Cells were removed from plates using Gibco® Versene solution and pelleted by centrifugation (Sorvall Super T21 high speed centrifuge, swinging bucket rotor) at 800 rpm for five minutes. Media was gently aspirated and cells were resuspended in Dulbecco's phosphate buffered saline solution (DPBS 1× [−] without calcium and magnesium chloride, Gibco ® Cat # 14190-144). A 10 μL aliquot of cell suspension was counted manually using a hemocytometer after addition of Trypan blue dye (BioRad). Cells were again pelleted by centrifugation, and DPBS was gently aspirated. The pelleted cells were then resuspended in a solution of freshly made stimulation buffer (Hank's Balanced Salt Solution [HBSS 10× [−] sodium bicarbonate] and [−] phenol red, Gibco®], 0.5 mM isobutylmethylxanthine [IBMX], 5 mM HEPES buffer solution [1M, Gibco®], 0.1% bovine serum albumin [BSA] in Milli-Q water, pH=7.4) and anti-cAMP acceptor beads (1.0 unit per well, AlphaScreen®). A cell/acceptor bead solution was added manually to each well of a 384 well microplate (OptiPlate-384; PerkinElmer) for final concentrations of 10,000 cells/well and 1.0 Unit anti-cAMP acceptor beads/well. The cells were then stimulated with ligand diluted in stimulation buffer to achieve their final concentrations in the well ranging from 10⁻¹³ to 10⁻⁴ M. The stimulated plates were incubated in a dark laboratory drawer at room temperature for two hours.

Meanwhile, a biotinylated cAMP/streptavidin donor bead working solution was made by adding biotinylated cAMP (1 Unit/well, AlphaScreen®) and streptavidin donor beads (1 Unit/well, AlphaScreen®) to a lysis buffer (10% Tween-20, 5 mM HEPES buffer solution [1M, Gibco®], 0.1% bovine serum albumin [BSA] in Milli-Q water, pH=7.4). After the two hour stimulation, the biotinylated cAMP/Streptavidin donor bead working solution was added to each well under green light and mixed well by pipetting up and down. The cells were incubated for another two hours at room temperature in a dark drawer at room temperature. The plate was then read on an EnSpire™ Alpha plate reader using a pre-normalized assay protocol set by the manufacturer. Assays were performed with duplicate data points on each plate and repeated in at least three independent experiments. Each plate contained a control ligand dose response (NDP-MSH, α-MSH, or γ₂-MSH), a 10⁻⁴M forskolin positive control, and a no ligand assay buffer negative control.

Dose response curves were analyzed using the PRISM program (v4.0; GraphPad Inc.). Potency EC₅₀ values (concentration that caused 50% maximal signal) were calculated by a nonlinear regression method. To be consistent with functional data being represented as an increasing response with increasing concentration and because the AlphaScreen® assay is a competition assay, a transformation was carried out for illustration purposes to normalize data to control compounds and flip dose response curves as previously described (Lensing, C. J. et al. J. Med. Chem. 2016, 59, 3112-3128 and Singh, A. et al. ACS Med. Chem. Lett. 2015, 6, 568-572).

### PRESO-Tango β-Arrestin Recruitment) Assay

The PRESTO-Tango assay was developed by Kroeze and coworkers for identifying biologically activate compounds by the rapid screening for most of the entire druggable GPCRome (Kroeze, W. K. et al. Nat. Struct. Mol. Biol. 2015, 22, 362-369 and Barnea, G. et al. Proc. Natl. Acad. Sci. U. S. A. 2008, 105, 64-69). The plasmids and assay technology was kindly provided by the Bryan Roth laboratory (University of North Carolina at Chapel Hill) and are now available through ADDGENE (Kit # 1000000068). Briefly, HTLA cells (HEK293 cells that stably express a tTA-dependent luciferase reporter and a β-arrestin 2—TEV fusion gene and were kindly provided by Richard Axel) (Barnea, G. et al. Proc. Natl. Acad. Sci. U. S. A. 2008, 105, 64-69) were maintained in DMEM supplemented with 10% FBS, 100 U/mL penicillin, 100 μg/mL streptomycin, 2 μg/mL puromycin, and 100 μg/mL hygromycin B in humidified atmosphere of 95% air and 5% CO₂ at 37° C.

The first day of the assay, HTLA cells were plated at approximately 1×10⁶ cells per 10 cm plate and grown to 20-40% confluency. The second day cells were transiently transfected using the calcium phosphate method with 4 μg/plate of hMC4R PRESTO-Tango plasmid construct and incubated 15-24 hours in humidified atmosphere of 97% air and 3% CO₂ at 35° C. (Kroeze, W. K. et al. Nat. Struct. Mol. Biol. 2015, 22, 362-369 and Chen, C. A. et al. Biotechniques 1988, 6, 632-638). The third day, cells were removed from 10 cm plates using Gibco® Versene solution and pelleted by centrifugation (Sorvall Super T21 high speed centrifuge, swinging bucket rotor) at 800 rpm for five minutes at room temperature. Cells were manually counted on a hemocytometer and resuspended in 1% dialyzed FBS and 1% penicillin/streptomycin in DMEM to a final concentration of 400,000 cells/mL. Cells were plated into 384-well white wall and clear bottom microplate (ViewPlate-384 TC, PerkinElmer Cat # 6007480) for a final concentration of 20,000 cells/well and incubated in 5% CO₂ at 37° C. On the fourth day, cells were stimulated by ligands diluted to the appropriate in well concentrations (i.e. 10⁻¹² to 10⁻⁵ M) in filter-sterilized assay buffer (20 mM HEPES, 1× HBSS, water, titrated to pH 7.4 with 1 N NaOH). Stimulated cells were incubated for 18 hours in 5% CO₂ at 37° C. On the fifth day, the assay buffer and cell medium was removed by aspiration. Then 20 μL of Bright-Glo (Promega, Cat # N1661) diluted 20-fold in assay buffer was added to each well and incubated to 15-20 minutes. After incubation, luminescence was then read on an EnSpire™ Alpha plate reader using a pre-normalized assay protocol for luminescence set by the manufacturer. Dose response curves were analyzed using the PRISM program (v4.0; GraphPad Inc.). Potency EC₅₀ values (concentration that caused 50% maximal signal) were calculated by a nonlinear regression method.

### ¹²⁵I-NDP-MSH Competitive Binding Affinity Studies:

NDP-MSH was radioiodinated with Na¹²⁵I utilizing the chloramine T procedure (26). Monoradioiodinated NDP-MSH (specific activity: 2175 Ci/mmol) was separated from uniodinated and diradioiodinated peptide by HPLC using a C₁₈ column eluted isocratically with 24% acetonitrile: 76% trimethylamine phosphate (pH 3.0) mobile phase. HEK293 cells stably expressing wildtype mMC1R or mMC4R were maintained as described above. Transiently transfected HEK293 cells were used for binding experiments on the mMC3R. Transfection was performed in 10 cm plates using FuGene6 transfection reagent (15 μL/plate; Promega), Opti-Mem medium (1.7 mL/plate; Invitrogen), and mMC3R-Flag DNA (3.33 μg/plate) two days prior to binding experiment. One or two days preceding the competition experiments, cell were plated into 12-well tissue culture plates (Corning Life Sciences, Cat. # 353043) and grown to 90-100% confluency. On the day of the assay, media was removed gently. The cells were treated with a freshly diluted aliquot of non-labeled compound at the in well concentration being tested (ranging from 10⁻¹² to 10⁻⁴ M as appropriate) in assay buffer (DMEM and 0.1% BSA) and a constant amount of ¹²⁵1-NDP-MSH (100,000 cpm/well) and were incubated at 37° C. for one hour. Media was gently aspirated and cells were washed gently once with assay buffer. The assay buffer was gently aspirated, and then cells were lysed with NaOH (500 μL; 0.1 M) and Triton X-100 (500 μL; 1%) for a minimum of 10 minutes. The cell lysate was transferred to 12×75 mm polystyrene tubes. The radioactivity was quantified on WIZARD² Automatic Gamma Counter (PerkinElmer). All experiments included unlabeled NDP-MSH as a positive control. All experiments were performed with duplicate data points and repeated in at least two independent experiments. The non-specific values used for calculations was radioactivity of the 10⁻⁶ M unlabeled NDP-MSH. Data was analyzed by a nonlinear regression method using the PRISM program (v4.0; GraphPad Inc.) to generate and calculate dose-response curves and IC₅₀ values. The standard error of the mean (SEM) was derived from the IC₅₀ values from at least two independent experiments.

### EXAMPLE 3

### Ligand Dependent Modulation of BRET Signal

Bioluminescence resonance energy transfer (BRET) has been routinely used to assess GPCR dimerization (Pfleger, K. D. et al. Nat. Protoc. 2006, 1, 337-345). Specifically, the MC3R and MC4R have been reported to result in high basal BRET signal supporting the formation of homodimers (Lensing, C. J. et al. ACS Chem. Neurosci. 2017, 8, 1262-1278; Kopanchuk, S. et al. Neurochem. Int. 2006, 49, 533-542; Mandrika, I. et al. Biochem. Biophys. Res. Commun. 2005, 326, 349-354 and Nickolls, S. A. et al. Peptides 2006, 27, 380-387). Furthermore, BRET has been utilized to support the existence of hMC1R-hMC3R and mMC3R-mMC4R heterodimers (Lensing, C. J. et al. ACS Chem. Neurosci. 2017, 8, 1262-1278 and Mandrika, I. et al. Biochem. Biophys. Res. Commun. 2005, 326, 349-354). It has also been suggested that ligand treatment can increase or decrease dimerization which should be detectable with changes in BRET signal (Tabor, A. et al. Sci. Rep. 2016, 6, 33233; Cottet, M. et al. Front. Endocrinol. (Lausanne) 2012, 3, 92; Grant, M. et al. J. Biol. Chem. 2010, 279, 36179-36183; Albizu, L. et al. Nat. Chem. Biol. 2010, 6, 587-594; Zheng, Y. et al. J. Med. Chem. 2009, 52, 247-258; Journe, A. S. et al. Medchemcomm 2014, 5, 792-796 and Russo, O. et al. J. Med. Chem. 50, 4482-4492). However, these reports vary depending on the receptor system and ligands used (Cottet, M. et al. Front. Endocrinol. (Lausanne) 2012, 3, 92). For example, agonist treatment at the somatostatin receptor 2 has been reported to cause the homodimers to dissociate into monomers (Grant, M. et al. J. Biol. Chem. 2010, 279, 36179-36183). Whereas at the vasopressin V₁ₐ receptor, agonist ligand had no observable effect on dimerization ratio (Albizu, L. et al. Nat. Chem. Biol. 2010, 6, 587-594). Also there are several examples of bivalent ligand treatment resulting increased BRET signal suggesting they are inducing or increasing dimerization (Tabor, A. et al. Sci. Rep. 2016, 6, 33233; Zheng, Y. et al. J. Med. Chem. 2009, 52, 247-258; Journe, A. S. et al. Medchemcomm 2014, 5, 792-796 and Russo, O. et al. J. Med. Chem. 50, 4482-4492). In previous reports focused melanocortin receptors, no significant effect of agonist or antagonist ligand was reported for the hMC1R, hMC3R, or hMC4R homodimerization (Mandrika, I. et al. Biochem. Biophys. Res. Commun. 2005, 326, 349-354 and Nickolls, S. A. et al. Peptides 2006, 27, 380-387). However, in the BRET study involving the hMC4R, there does appear to be a trend towards decreasing BRET signal after agonist dosing, albeit not significant. After dosing α-MSH at 1 μM the mean BRET signal decreased by approximately 20% compared to basal BRET signal of the hMC4R (Nickolls, S. A. et al. Peptides 2006, 27, 380-387). Because of the potential of the compounds to be modulating the dimer or oligomer state or changing the dimer conformational state, we investigated the response of BRET signal from mMC4R in response to ligand treatment (FIG. 2D).

Ligands α-MSH, CJL-1-14, and CJL-1-87, that have full agonist activity in both the cAMP signaling assay and the β-arrestin recruitment assay, resulted in a dose dependent decrease in BRET signal (FIG. 2D). Dosing these ligands at 10 μM resulted in a significant 15% reduction in BRET signal compared to basal signal. In contrast, ligands CJL-1-80 and CJL-1-140 contain only the antagonist tetrapeptide scaffold and have minimal functional agonist activity in both the cAMP signaling assay and the β-arrestin recruitment assay. These antagonist-based ligands resulted in no significant changes in BRET signal from basal levels at the concentrations assayed. In addition, the equal tetrapeptide mixture of agonist CJL-1-14 and antagonist CJL-1-80 resulted in no significant changes from basal signal. The MUmBLs, CJL-1-124 and CJL-5-58, resulted in a significant effect in which dosing 10 μM resulted in approximately an 8% reduction in BRET signal compared to basal signal (FIG. 2D).

The reduction of BRET signal observed with agonist containing ligands could be the result of two different mechanisms: 1) The dimerization or oligomerization is being disrupted and moving towards a lower oligomer state (e.g. dimers to monomers). 2) A conformational change is occurring within the intact dimer or higher-order oligomer in which the NanoLuc®-donor and the HaloTag®-acceptor are being orientated such that the BRET signal is being reduced (e.g. moving further away or dipole orientation is incorrect) (Broussard, J. A. et al. Nat. Protoc. 2013, 8, 265-281). It is currently difficult to determine which of these two possibilities are the driving force for the BRET signal reduction observed in our studies. Regardless, it is apparent that some sort of conformational change is occurring that effects the BRET signal that relates with ligands agonist activity both for cAMP and for β-arrestin recruitment.

These changes match the proposed asymmetric signaling of MC4R homodimers. It follows from the proposed model that at basal levels in which only assay buffer is added (FIG. 3, state A), no conformational changes have occurred. With the addition of agonist ligand and the first binding event, cAMP signaling pathways are activated and a conformational change occurs that effects BRET signal (c.a. 7-8% change) (FIG. 3, state B or E). This is observed with all ligands that contain an agonist scaffold including α-MSH, CJL-1-14, CJL-1-87, CJL-1-124, and CJL-5-58 (FIG. 2D). The second agonist binding event is hypothesized to result in an additional conformational change at the second receptor in the homodimer, and this is postulated to be responsible for the maximal observed decrease in BRET signal (c.a. 15%) (FIG. 3, state F). This is observed with ligands α-MSH, CJL-1-14, and CJL-1-87 because they result in the second conformational change with in the homodimer due to a second agonist binding event on the second receptor. However, the second receptor in the homodimer pair is postulated to be bound by an antagonist scaffold with ligands CJL-5-58 and CJL-1-124 (FIG. 1C) and, therefore, the full conformational change to the homodimer does not occur (FIG. 3, state I) resulting in the lack of β-arrestin recruitment (FIG. 2B-2C) and the only 50% maximal change in BRET signal (i.e. 7-8% change instead of 15%) (FIG. 2D).

The current studies support the hypothesis that the bias agonism observed currently with CJL-5-58 is the result of a conformational change of the dimeric state as correlated with the changes observed in the BRET signal. These conformational changes could be changes in the oligomeric number (e.g. dimers to monomers), orientation of the receptors within a dimer pair (e.g. which transmembrane helixes are interacting), or changes in the cellular location of the receptors (e.g. receptor internalization) (Akgün, E. et al. J. Med. Chem. 2015, 58, 8647-8657; Chapman, K. L. et al. Biochim. Biophys. Acta. 2013, 1828, 535-542 and Piechowski, C. L. et al. J. Mol. Endocrinol. 2013, 51, 109-118).

### Bioluminscence Resonance Energy Transfer (BRET) Studies

The NanoBRET™ Protein:Protein Interaction System was utilized according to manufacturer's instructions to examine the association and proximity of the melanocortin receptors as previously reported (Lensing, C. J. et al. ACS Chem. Neurosci. 2017, 8, 1262-1278). Briefly, the plasmids were constructed to incorporate the NanoLuc® fusion protein and the HaloTag® fusion protein onto the C-terminus the mMC4R of the plasmids described above. Proper cell membrane expression and ligand binding have previously been supported by competitive binding experiments (Lensing, C. J. et al. ACS Chem. Neurosci. 2017, 8, 1262-1278). The specificity of signal has also previously been shown (Lensing, C. J. et al. ACS Chem. Neurosci. 2017, 8, 1262-1278). On the first day, cells were plated into 6 well plates in the morning. In the afternoon of the same day, cells were transiently transfected with mMC4R-NanoLuc® fusion protein and the MC4R-HaloTag® fusion protein by adding FuGene6 Transfection (8 μL/well, Promega), DNA (2 μg/well) in OptiMem medium (Invitrogen) at a total volume of 100 μL/well. The ratio of donor NanoLuc® to acceptor HaloTag® DNA has previously been optimized and a ratio of 1 Receptor-NanoLuc® plasmid: 4 Receptor-HaloTag® plasmid was utilized for all experiments (Lensing, C. J. et al. ACS Chem. Neurosci. 2017, 8, 1262-1278). Cells were incubated with transfection reagent overnight at 5% CO₂ at 37° C. One day after the transfection, cells were re-plated into 96-well black clear bottom plates (Cat # 3603, Corning Life Sciences) at 30,000 cells in 90 μL of assay buffer (4% FBS in OptiMem).

To each well, 1 μL of 0.1 mM HaloTag® NanoBRET™ 618 ligand was added and incubated 18-24 h at 5% CO₂ at 37° C. As a negative control, each assay also included; “no acceptor controls” in which 1 μL of DMSO was added instead of 618 ligand rendering the BRET relay system incomplete. This provides the background signal and was subtracted from the final experimental signal. Plates were then developed 48 to 60 hours after transfection. Two hours before the plates were developed, 10 μL of a 10× aliquot of the ligand diluted in assay buffer was added to each well to yield the final in well concentration (10⁻⁵, 10⁻⁷, or 10⁻⁹M) of each compound. For the assay buffer control, 10 μL of assay buffer was added instead of compound. To develop plates, 25 μL of 5× solution of NanoBRET™ Nano-Glo® Substrate in Opti-MEM® was added to each well. Plates were then read within 10 min on a FlexStation® 3 plate reader (Molecular Devices) at the donor emission wavelength (460 nm) and acceptor emission wavelength (618 nm). The milli BRET Units (mBUs) were calculated by dividing the acceptor emission of 618 nm by the donor emission at 460 nm and multiplying it by 1000. The standard error of the mean (SEM) was derived from at least three independent experiments.

### EXAMPLE 4

### Food Intake after Administration of CJL-5-58 in Wild Type Mice

The novel in vitro pharmacological profile of the MUmBLs warranted further evaluation in vivo to study their effects on energy homeostasis and physiological consequences. In particular, compound CJL-5-58 was selected due to its biased agonism at the hMC4R, consistent pharmacology in cAMP accumulation assays between the mouse and human isoforms, and the increased serum stability of a PEDG20 linker compared to a Pro-Gly linker (Lensing, C. J. et al. ACS Chem. Neurosci. 2017, 8, 1262-1278). The compound was administered intracerebroventricularly (ICV) directly into the lateral ventricle of the brain in order to avoid the confounding effects of metabolism and brain delivery and to be consistent with previous work in the field (Lensing, C. J. et al. J. Med. Chem. 2016, 59, 3112-3128; Lensing, C. J. et al. ACS Chem. Neurosci. 2017, 8, 1262-1278; Irani, B. G. et al. Eur. J. Pharmacol. 2011, 660, 80-87; Marsh, D. J. et al. Nat. Genet. 1999, 21, 119-122; Ericson, M. D. et al. Biochim. Biophys. Acta, Mol. Basis Dis. 2017, 1863, 2414-2435 and Lensing, C. J. et al. ACS Chem. Neurosci. 2016, 7, 1283-1291).

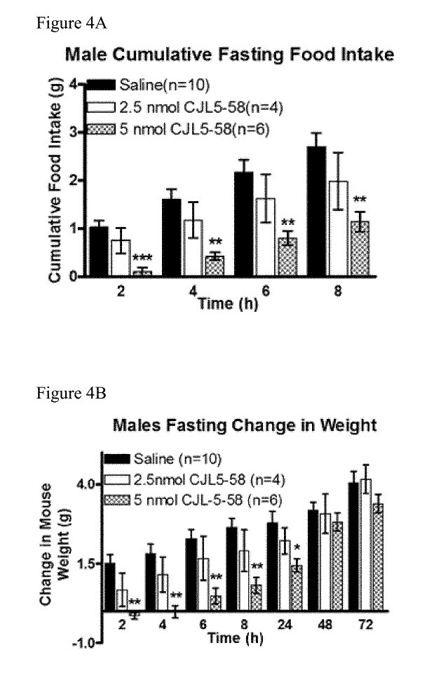

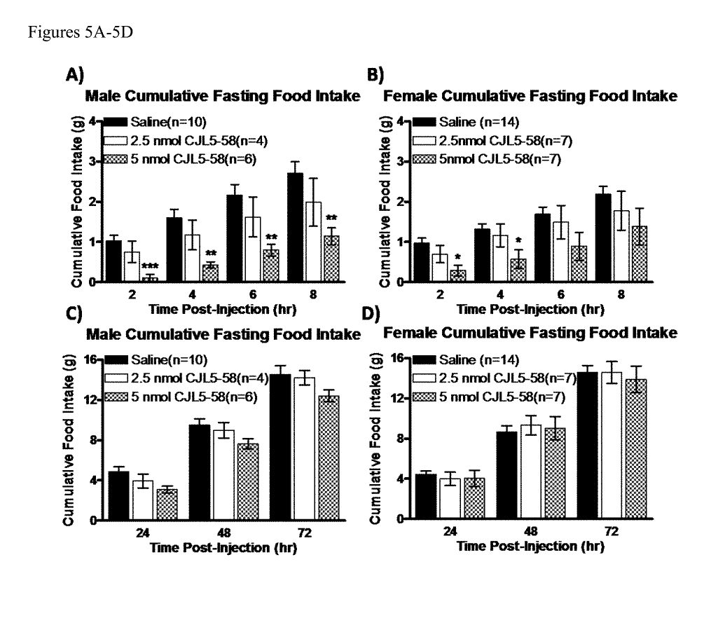

Dose response studies were performed in “conventional” standard mouse cages in which all measurements were taken manually. Compound CJLS-58 resulted in no signs of adverse effects at doses of 2.5 nmols and 5.0 nmols in the conventional cage experiments. Compound CJL-5-58 resulted in a dose dependent decrease in food intake when refeeding was measured after a 22 hour fast. Significant decreased food intake was observed at 2, 4, 6, and 8 hours after compound administration in male mice (FIG. 4A), and 2 and 4 hours in female mice. Consistent with the decreased food intake, male mice receiving CJL-5-58 in the fasting refeeding paradigm did not return to their pre-fasting weights as quickly as the saline controls. A significant difference was observed in the change in weight of the male mice after compound administration at time points 2, 4, 6, 8, and 24 hours after compound administration (FIG. 4B). Only the 2 hour time point was significant in female mice.

No statistically significant effect on either food intake or body weight was observed with CJL-5-58 at a 5.0 nmol dose in a nocturnal free-feeding paradigm. In the nocturnal feeding paradigm, mice have free access to food the entire course of the experiments, and compound is administered 2 hours before lights out (Lensing, C. J. et al. J. Med. Chem. 2016, 59, 3112-3128; Lensing, C. J. et al. ACS Chem. Neurosci. 2017, 8, 1262-1278 and Lensing, C. J. et al. ACS Chem. Neurosci. 2016, 7, 1283-1291). Since mice consume approximately 70% of their food during the dark cycle with their biggest meal being soon after lights out, this paradigm should measure the effect on food intake with minimal disruption of homeostasis (Ellacott, K. L. et al. Cell Metab. 2010, 12, 10-17). However, in the fasting-refeeding paradigm, mice are fasted from the start of the previous dark cycle until 2 hours before lights out. At which point mice were administered the compound and food was returned. This disrupts the normal homeostasis of the mice by putting them in a fasting state, but the fast creates a robust feeding response that can help to detect significant effects (Lensing, C. J. et al. ACS Chem. Neurosci. 2017, 8, 1262-1278 and Ellacott, K. L. et al. Cell Metab. 2010, 12, 10-17). Specific to the melanocortin system, expression of endogenous antagonist AGRP is upregulated (Haskell-Luevano, C. et al. Endocrinology 1999, 140, 1408-1415; Marsh, D. J. et al. Brain Res. 1999, 848, 66-77 and Hahn, T. M. et al. Nat. Neurosci. 1998, 1, 271-272).

CJL-5-58's potent binding affinity (IC₅₀=14 nM) may allow it to compete more effectively against endogenous ligands for binding. In the fasting paradigm, compound CJL-5-58 is directly competing with agouti-related peptide (AGRP) which is an endogenous MC3R/MC4R antagonist whose expression levels are upregulated during fasting (Haskell-Luevano, C. et al. Endocrinology 1999, 140, 1408-1415; Marsh, D. J. et al. Brain Res. 1999, 848, 66-77 and Hahn, T. M. et al. Nat. Neurosci. 1998, 1, 271-272). It, therefore, may be hypothesized that CJL-5-58 achieves its effects in the fasting state by blocking the orexigenic effects of AGRP. Regardless, the agonist pharmacophore is overriding the antagonist pharmacophore in the regulation of food intake behavior in vivo.

In order to better characterize the effects of CJL-5-58 on energy homeostasis, it was decided to perform compound administration in TSE Phenotypic metabolic cages that are configured to automatically measure water intake, food intake, changes in the CO₂ and O₂ levels within the cages, and beam break activity in wild type mice, MC3RKO mice, and MC4RKO mice. In these experiments, a similar trend of CJL-5-58 acting as an agonist by reducing food intake was observed. In must also be noted for full disclosure and good scientific practices that some adverse behaviors were observed during the metabolic cage experiments. These adverse reactions did not appear to affect the parameters measured (i.e. activity, food intake, water intake, RER, and energy expenditure). Also, the behaviors were different depending on the housing conditions and experimental paradigm utilized suggesting that they may not necessarily be compound specific.

### Animals

All experiments were performed in accordance with the Institutional Animal Care and Use Committee (IACUC) at the University of Minnesota. Female and male littermates with mixed background from C57BL/6J and 129/Sv inbred strains were 8 weeks old at the time of surgeries. Mice were individually housed after surgeries and for the remainder of the experiment. Mice were maintained on a 12 hr light/dark cycle (Lights off was at 11:59 AM) in a temperature controlled room (23-25° C.). In the nocturnal feeding paradigm, mice had free access to normal chow (Harlan Teklad 2018 Diet: 18.6% crude protein, 6.2% crude fat, 3.5% crude fiber, with energy density of 3.1 kcal/g). In the fasting-refeeding paradigm, mice were fasted from lights out on the previous day until compound administration 2 hours prior to lights out for a total fasting time of 22 h (Lensing, C. J. et al. ACS Chem. Neurosci. 2017, 8, 1262-1278 and Ellacott, K. L. et al. Cell Metab. 2010, 12, 10-17). Mice had free access to tap water throughout all the experiments. The cannula placement validation studies and conventional cage studies were performed in standard mouse polycarbonate conventional cages provided by University of Minnesota's Research Animal Resources (RAR). Weekly cage changes were conducted by lab research staff.

### Cannulation Surgeries and Placement Validation Studies

A cannula was surgically placed into the lateral cerebral ventricle as previously reported (Lensing, C. J. et al. J. Med. Chem. 2016, 59, 3112-3128; Lensing, C. J. et al. ACS Chem. Neurosci. 2017, 8, 1262-1278; Irani, B. G. et al. Eur. J. Pharmacol. 2011, 660, 80-87 and Lensing, C. J. et al. ACS Chem. Neurosci. 2016, 7, 1283-1291). Mice were anesthetize with a mixture of ketamine (100 mg/kg) and xylazine (5 mg/kg) and were positioned in a stereotaxic apparatus (David Kopf Instruments). A 26-gauge cannula (Cat# 81C315GS4SPC; PlasticsOne, Roanoke, Va.) was inserted in the lateral cerebral ventricle at the coordinates 1.0 mm lateral and 0.46 mm posterior to bregma and 2.3 mm ventral to the skull (Franklin, K. B. J. & Paxinos, G. The Mouse Brain in Stereotaxic Coordinates. (Academic Press, 1997)). The cannula was secured to the skull using dental cement (C&B-Metabond Adhesive cement (Kit # S380) followed by Lang's Jet™ Denture Repair Kit (Jet Denture Repair Powder Ref #1220; Jet Liquid Ref # 1403). A post-surgery dose of flunixin meglumine (FluMegluine, Clipper Distribution Company) and 0.5 mL of 0.9% saline (Hospira, Lake Forrest, Ill.) was given subcutaneously to aid in surgery recovery. All mice were given at least seven days to recover from surgery before any treatment.

Cannula placement was verified by evaluating the feeding response after ICV administration of 2.5 μg of human (h)PYY₃₋₃₆ (Bachem Cat # H-8585) as described previously (Lensing, C. J. et al. J. Med. Chem. 2016, 59, 3112-3128; Lensing, C. J. et al. ACS Chem. Neurosci. 2017, 8, 1262-1278; Irani, B. G. et al. Eur. J. Pharmacol. 2011, 660, 80-87; Marsh, D. J. et al. Nat. Genet. 1999, 21, 119-122 and Lensing, C. J. et al. ACS Chem. Neurosci. 2016, 7, 1283-1291). Each mouse was administered hPYY and saline on different days following a crossover design in the nocturnal feeding paradigm. In the nocturnal paradigm, compound is administered 2 hours prior to lights out with free access to food and water throughout the entire experiment. There was at least a 4 day washout period between administration to ensure that normal feeding patterns and body weight returned. Food intake and body weight were manually measured 2, 4, and 6 hours after hPYY or saline administration. A mouse with a validated cannula placement for the TSE cage experiments consumed at least 1.0 g more after hPYY administration compared to saline administration 4 hours post-administration.

### CJL-5-58 Administration and Energy Homeostasis Studies

As stated above, conventional cage experiments were performed in standard mouse polycarbonate conventional cages. The indicated amount (nmols) of compound in 3 μL of saline was delivered two hours prior to lights out (t=0 hr) through the implanted cannula using an infusion internal cannula (Cat# 8IC315IS4SPC; PlasticsOne, Roanoke, Va.) in either a satiated nocturnal feeding paradigm or a fasting refeeding paradigm as described above (Lensing, C. J. et al. ACS Chem. Neurosci. 2017, 8, 1262-1278 and Ellacott, K. L. et al. Cell Metab. 2010, 12, 10-17). All experiments followed a cross-over paradigm in which the mouse received saline and compound on different days with a washout period in between each treatment. Statistical analysis was performed using SPSS V23 software (IBM) using a multivariate general linear model followed by a Bonferroni's post hoc test. Results are presented as Mean±SEM. Statistically significant was considered p<0.05.

### Discussion

There is a growing amount of evidence that GPCR homodimers are functionally relevant and are pharmaceutical targets. A broadly applicable drug design strategy that targets homodimers, as opposed to monomeric receptors, would theoretically double the amount GPCR drug targets. Although various labs have presented different techniques and proof of concepts for methods to target asymmetrically signaling GPCR homodimers (Han, Y. et al. Nat. Chem. Biol. 2009, 5, 688-695; Pellissier, L. P. et al. J. Biol. Chem. 2011, 286, 9985-9997; Teitler, M. et al. Pharmacol. Ther. 2012, 133, 205-217; Comps-Agrar, L. et al. EMBO J. 2011, 30, 2336-2349; Pin, J. P. et al. Febs J. 1 2005, 272, 2947-2955; Hlavackova, V. et al. EMBO J. 2005, 24, 499-509; Prezeau, L. et al. Neuropharmacology 2005, 49, 267-267; Kniazeff, J. et al. Nat. Struct. Mol. Biol. 2004, 11, 706-713; Kniazeff, J. et al. J. Neurosci. 2004, 24, 370-377; Zylbergold, P. et al. Nat. Chem. Biol. 2009, 5, 608-609; Szalai, B. et al. Biochem. Pharmacol. 2012, 84, 477-485; Damian, M. et al. EMBO J. 2006, 25, 5693-5702; Brock, C. et al. J. Biol. Chem. 2007, 282, 33000-33008; Sartania, N et al. Cell. Signal. 2007, 19, 1928-1938 and Gracia, E. et al. Neuropharmacology 2013, 71, 56-69), these techniques would be difficult to adapt to therapeutic design and in vivo applications. The instant invention presents a design strategy that targets asymmetric homodimers that should be easily amendable to various GPCR systems and in vivo targeting applications.

The MUmBL design strategy aims at occupying each of the two receptors within the homodimer with a different pharmacophore such that an agonist pharmacophore and an antagonist pharmacophore each occupy one of the two receptors within each homodimer. This design strategy produced biased ligands at the hMC4R in which the cAMP signaling pathway was robustly activated at nanomolar concentrations (EC₅₀˜2 to 6 nM) but the β-arrestin pathway was only partially activated at a concentration of 10 μM. These are the first melanocortin biased ligands favoring cAMP signaling over β-arrestin recruitment and will be valuable chemical probes to study melanocortin signaling in the disease states and disorders in which the melanocortin receptors are implicated including: cancer (Xu, L. P. et al. Proc. Natl. Acad. Sci. U. S. A. 2012, 109, 21295-21300; Josan, J. S. et al. Bioconjugate Chem. 2011, 22, 1270-1278; Barkey, N. M. et al. J. Med. Chem. 2011, 54, 8078-8084; Brabez, N. et al. ACS Med. Chem. Lett. 2013, 4, 98-102 and Brabez, N. et al. J. Med. Chem. 2011, 54, 7375-7384), skin pigmentation disorders (Langendonk, J. G. et al. N. Engl. J. Med. 2015, 373, 48-59), social disorders (Penagarikano, O. et al. Sci. Transl. Med. 2015, 7, 271 and Barrett, C. E. et al. Neuropharmacology 2014, 85, 357-366), sexual function disorders (Uckert, S. et al. Expert Opin. Invest. Drugs 2014, 23, 1477-1483; Clayton, A. H. et al. Women's Health 2016, 12, 325-337 and Kingsberg, S. et al. J. Sex. Med. 2015, 12, 389-389), Alzheimer's disease (Giuliani, D. et al. Mol. Cell. Neurosci. 2015, 67, 13-21 and Giuliani, D. et al. Neurobiol. Aging 2014, 35, 537-547), cachexia (Joppa, M. A. et al. Peptides 2005, 26, 2294-2301; Deboer, M. D. et al. Trends Endocrinol. Metab. 2006, 17, 199-204; Doering, S. R. et al.ACS Med. Chem. Lett. 2015, 6, 123-127 and Ericson, M. D. et al. J. Med. Chem. 2015, 58, 4638-4647), and obesity (Lensing, C. J. et al. J. Med. Chem. 2016, 59, 3112-3128; Irani, B. G. et al. Eur. J. Pharmacol. 2011, 660, 80-87; Marsh, D. J. et al. Nat. Genet. 1999, 21, 119-122 and Fan, W. et al. Nature 1997, 385, 165-168).

Two of the compounds showed species difference in which a partial agonist dose response curve was observed at the mMC4R. This identified CJL-5-58 as the lead ligand for in vivo evaluation due to its biased agonism at the hMC4R, consistent pharmacology in cAMP signaling assays between the mouse and human receptors, and the increased serum stability of a PEDG20 linker compared to a Pro-Gly linker (Lensing, C. J. et al. ACS Chem. Neurosci. 2017, 8, 1262-1278). Evaluation in vivo showed that CJL-5-58 reduce food intake after administration in a fasting-refeeding paradigm consistent with its cAMP agonist function.

The UmBL methodology presented currently should be applicable to various other GPCRs and can easily accommodate the plethora of well-studied and developed selective agonists and antagonists for various GPCR systems. This bivalent ligand targeting method should allow for biased ligands or unique pharmacologies at various receptors by combining known agonists and antagonists with an effective linker. Considering the wide array of GPCRs that are already reported to exist as allosterically modulated or asymmetric homodimers (including the vasopressin (Orcel, H. et al. Mol. Pharmacol. 2009, 75, 637-647), dopamine (Han, Y. et al. Nat. Chem. Biol. 2009, 5, 688-695), adenosine (Gracia, E. et al. Neuropharmacology 2013, 71, 56-69), metabotropic glutamate (Kniazeff, J. et al. Nat. Struct. Mol. Biol. 2004, 11, 706-713), and serotonin receptors (Pellissier, L. P. et al. J. Biol. Chem. 2011, 286, 9985-9997)) this strategy should be broadly applicable. In order to effectively synthesize UmBLs for other receptor systems, it will be necessary to perform some standard medicinal chemistry to optimize the connection points of the linker to the pharmacophores, optimize the linker properties, and optimize the orientation of the pharmacophores. Based on studies at the mouse melanocortin receptors, it was desirable if the agonist scaffold and the antagonist scaffold had approximately equal binding affinities.

The exact pharmacology that may be achieved through the UmBLs design strategy will be as diverse as the allosteric mechanisms between different GPCR homodimers. For example, based on the results of Han and coworkers it can be hypothesized that UmBLs targeting the dopamine D2 receptor would result in increased receptor activation beyond just monovalent agonist alone. This is because allosteric cross-talk of a second agonist protomer was shown to blunt activation, so the occupation of the second protomer with an antagonist scaffold instead of an agonist scaffold should increase signal activation (Han, Y. et al. Nat. Chem. Biol. 2009, 5, 688-695). In contrast if the UmBL approach was applied to the metabotropic glutamate receptor, it would be hypothesized to result in lower than full receptor activation of agonist alone as Kniazeff and coworkers observed that one agonist can partially activate a dimeric unit but two agonists are required for full activation (Kniazeff, J. et al. Nat. Struct. Mol. Biol. 2004, 11, 706-713). Finally, it has been reported that the vasopressin Vib receptor signals through both the G_{q/11}-inositol phosphate (IP) and the cAMP pathways (Orcel, H. et al. Mol. Pharmacol. 2009, 75, 637-647). It was hypothesized by Orcel and coworkers, that “the IP pathway could be activated by the binding of either one or two AVP molecules to a single receptor dimer. . . By contrast, cAMP production could only be turned on upon the binding of two ligands to a dimer.” Their observations and hypothesis is consistent with asymmetric homodimers such that the IP pathway is activated by the first agonist binding event and the cAMP pathway is activated second (Orcel, H. et al. Mol. Pharmacol. 2009, 75, 637-647). Therefore, if the UmBL design strategy was applied to ligands targeting the vasopressin V_{1b} receptor, it would be predicted to result in biased ligands in which the agonist pharmacophore would activate the IP pathway, and the antagonist pharmacophore would block the cAMP pathway activation within the homodimer. The UmBL design approach could also be applied to GPCR systems in which asymmetry between homodimers has not been identified, or even systems in which homodimerization has not yet been observed. In these situations, designed UmBLs could be evaluated for their ability to induce signaling in multiple signaling pathways (e.g. cAMP, Ca⁺, kinase signaling, β-arrestin signaling, ect.) to identify asymmetrically signaling GPCR homodimers.

### EXAMPLE 5

### Preliminary Food Intake Studies of CJL-5-58 in Mice

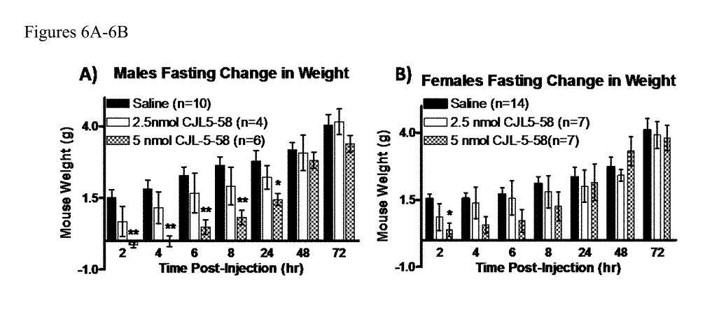

The initial dose response studies were performed in “conventional” standard mouse cages in which all measurements were taken manually. Compound CJL-5-58 resulted in no signs of adverse effects at doses of 2.5 nmols and 5.0 nmols in the conventional cage experiments. Compound CJL-5-58 resulted in a dose dependent decrease in food intake when refeeding was measured after a 22 hour fast. Significant decreases in food intake were observed at 2, 4, 6, and 8 hours after compound administration in male mice (FIG. 5A), and 2 and 4 hours in female mice (FIG. 5B). Consistent with the decreased food intake, male mice receiving CJL-5-58 in the fasting refeeding paradigm did not return to their pre-fasting weights as quickly as the saline controls. A significant difference was observed in the change in weight of the male mice after compound administration at time points 2, 4, 6, 8, and 24 hours after compound administration (FIG. 6A). Only the 2 hour time point was significant in female mice (FIG. 6B).

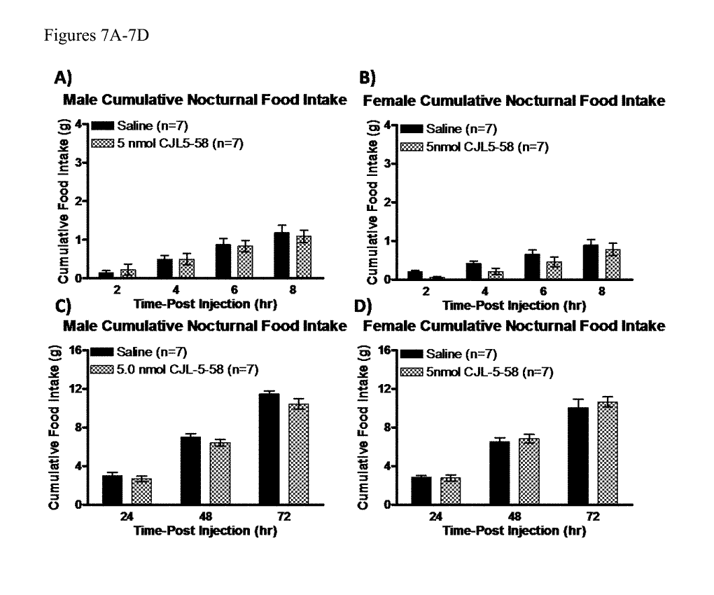

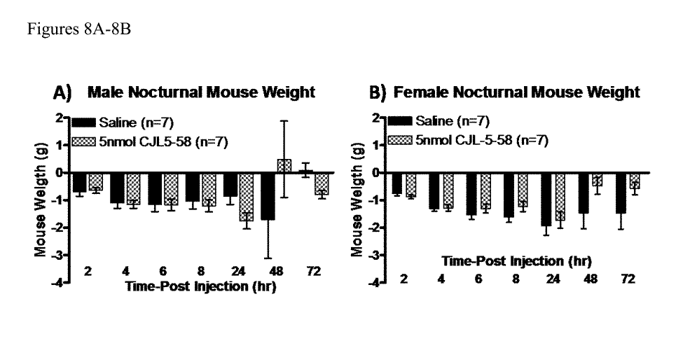

Interestingly, no statistically significant effect on either food intake or body weight was observed with CJL-5-58 at a 5.0 nmol dose in a nocturnal free-feeding paradigm (FIGS. 7A-7D and FIGS. 8A-8B). In the nocturnal feeding paradigm, mice have free access to food the entire course of the experiments, and compound is administered 2 hours before lights out (Lensing, C. J. et al. J. Med. Chem. 2016, 59, 3112-3128; Lensing, C. J. et al. ACS Chem. Neurosci. 2017, 8, 1262-1278 and Lensing, C. J. et al. ACS Chem. Neurosci. 2016, 7, 1283-1291). Since mice consume approximately 70% of their food during the dark cycle with their biggest meal being soon after lights out, this paradigm should measure the effect on food intake with minimal disruption of homeostasis (Ellacott, K. L. et al. Cell Metab. 2010, 12, 10-17). However, in the fasting-refeeding paradigm, mice are fasted from the start of the previous dark cycle until 2 hours before lights out. At which point mice were administered the compound and food was returned. This disrupts the normal homeostasis of the mice by putting them in a fasting state, but the fast creates a robust re-feeding response that can help to detect significant effects (Lensing, C. J. et al. ACS Chem. Neurosci. 2017, 8, 1262-1278 and Ellacott, K. L. et al. Cell Metab. 2010, 12, 10-17). Specific to the melanocortin system, expression of endogenous antagonist AGRP is upregulated upon fasting (Marsh, D. J. et al. Brain Res. 1999, 848, 66-77; Haskell-Luevano, C. et al. Endocrinology 1999, 140, 1408-1415 and Hahn, T. M. et al. Nat. Neurosci. 1998, 1, 271-272).

It is possible that CJL-5-58 achieves its effects in the fasting state by blocking the orexigenic effects of AGRP, and not from melanocortin agonist action. Indeed, food intake after CJL-5-58 is consistent between the nocturnal paradigm and the fasting paradigm suggesting that it maintains consistent feeding patterns regardless of endogenous homeostasis regulation.

### EXAMPLE 6

### Effects of CJL-5-58 In Vivo

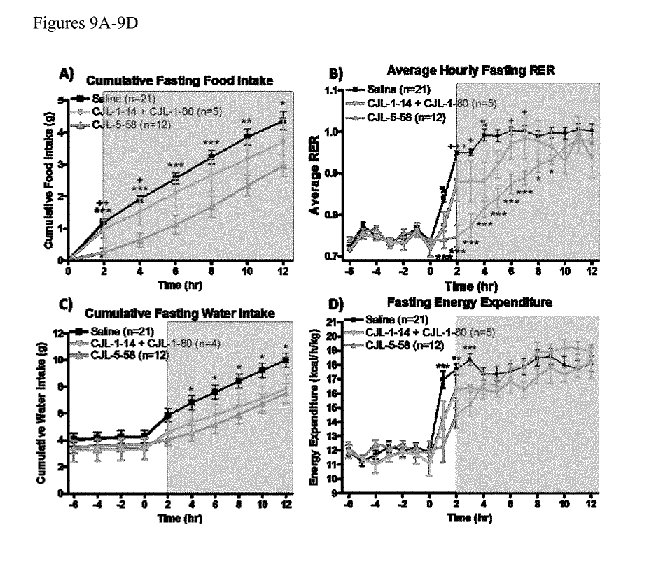

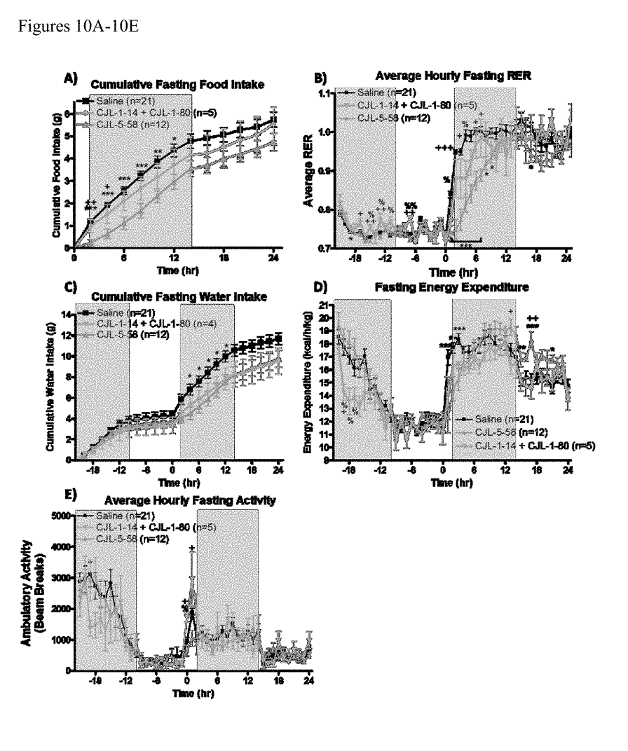

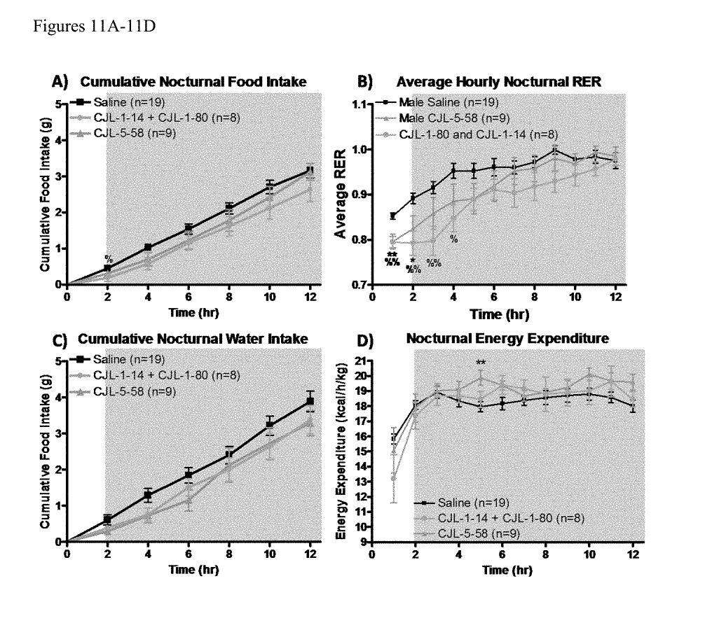

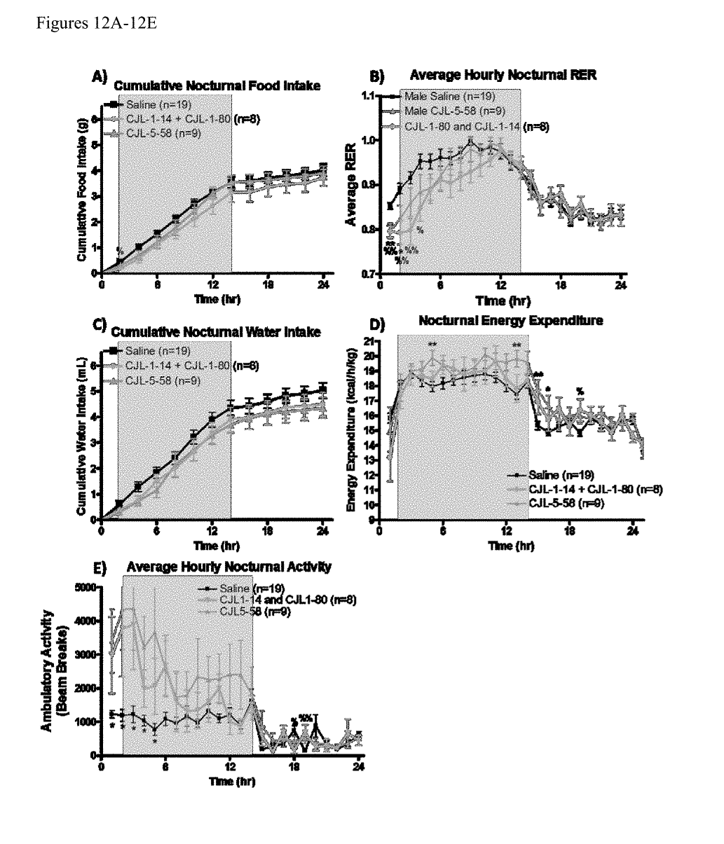

In order to better characterize the effects of CJL-5-58 on energy homeostasis, it was decided to perform compound administration in TSE Phenotypic metabolic cages that are configured to automatically measure water intake, food intake, changes in the CO₂ and O₂ levels within the cages, and beam break activity. A new cohort of littermate age match male mice was cannulated and acclimated to the TSE metabolic cages for one week. Consistent with the conventional cage data, the administration of 5 nmols of CJL-5-58 resulted in a decrease in food intake up to 12 hours after administration in the fasting paradigm (FIG. 9A). Because no consistent long term effects (>24 hours) were observed at any parameters measured, the discussion will be focused on the first 24 hours, with the majority of effects observed within the first 12 hours. (For full time course, see FIGS. 10A-10B). A decrease in water intake was observed with 5 nmols of CJL-5-58 at time points 4, 6, 8, 10, and 12 hours after compound administration in the fasting paradigm (FIG. 9C). This was not surprising since water intake correlates directly to food intake and is known to decline during fasting paradigms (Ellacott, K. L. et al. Cell Metab. 2010, 12, 10-17). It is, therefore, difficult in the current study to know if the decrease in water intake is a consequence of decreased food intake after CJL-5-58 administration or a direct pharmacological effect. As observed in the conventional cages, no significant effect was observed with the administration of CJL-5-58 in the nocturnal feeding paradigm (FIG. 11A, FIG. 12A). Also no difference in water intake was observed in the nocturnal paradigm (FIG. 11C).

Melanocortin ligands have previously been reported to effect the respiratory exchange ratio (RER), with agonist compounds decreasing the RER, and antagonist compounds increasing the RER. The RER can be measured indirectly utilizing TSE metabolic cage system by measuring the amount of CO₂ and O₂ entering and exiting the sealed cages. A RER value of c.a. 0.7 gives an indication thatfats are the primary fuel source that the animal is utilizing. A RER value of c.a. 1.0 gives an indication that carbohydrates are the primary fuel source the animal is utilizing. During the fast a baseline RER value of slightly above 0.7 was observed (FIG. 9B, FIG. 10B). This value has been reported previously in the literature (Lensing, C. J. et al. ACS Chem. Neurosci. 2017, 8, 1262-1278; Tanner, J. M. et al. Exp. Biol. Med. 2010, 235, 1489-1497 and Marvyn, P. M. et al. Data in brief2016, 7, 472-475), and is purportedly due to the lack of carbohydrates available for energy during a fast that results in a reliance of fat storage as the primary energy source. After saline treatment and the return of food, the RER value increases rapidly towards 1.0 as the mice consume food and the consumed carbohydrates become the primary fuel source through the first dark cycle. Administration of CJL-5-58 resulted in more gradual increase in RER from the 0.7 baseline value to 1.0. A significantly lower RER was observed for the first 9 hours and at 17 hours after compound CJL-5-58 administration compared to saline (FIG. 10B). The lowered RER values support a hypothesis that in addition to eating less, the mice were burning more fats instead of carbohydrates. In the nocturnal feeding paradigm, compound administration resulted in significantly lowered RER values only until 2 hours after administration supporting a hypothesis that the pharmacological effect of CJL-5-58 is amplified by fasting (FIG. 11B, FIG. 12B).

Melanocortin ligands have been reported to effect the energy expenditure such that agonists increase the amount of calories burned, and antagonist decrease the amount of calories burned. In the fasting paradigm, the energy expenditure decreases rapidly during fasting which is consistent with the mice conserving energy (FIG. 9D). The baseline energy expenditure is c.a. 12 kcal/h/kg prior to treatment. Following saline treatment and the reintroduction of food, energy expenditure increases rapidly in rate up to c.a. 17-19 kcal/h/kg. After the administration of the 5 nmol dose of CJL-5-58, energy expenditure also increases to c.a. 17-19 kcal/h/kg, however, the increase is more gradual and the energy expenditure remains significantly lower for the first 3 hours post-treatment. The mice's rate of energy expenditure eventually recovered and an increased energy expenditure is observed 15, 17, and 21 hours after compound administration compared to saline (FIG. 9D). This is interesting, because all other parameters are consistent with CJL-5-58 functioning as an agonist in vivo, but CJL-5-58 effects on energy expenditure is consistent with it functioning as an antagonist. The decrease in energy expenditure may be due to the robust decrease in food intake that keeps the mice in an energy conservative state.

Another hypothesis for the lowered energy expenditure may be the biased signaling of CJL-5-58 for the cAMP signaling pathway over the β-arrestin recruitment pathway. It could be hypothesized that the β-arrestin pathway is responsible for classic agonist effect to increase energy expenditure. Therefore, CJL-5-58, with minimal β-arrestin recruitment, results in a more gradual change in energy expenditure from baseline. However, further experimentation is necessary before hypothesis could be validated. In the nocturnal paradigm, an increase in energy expenditure was observed 5, 13, 15, and 16 hours after treatment which is consistent with agonist function (FIG. 11D, FIG. 12D).

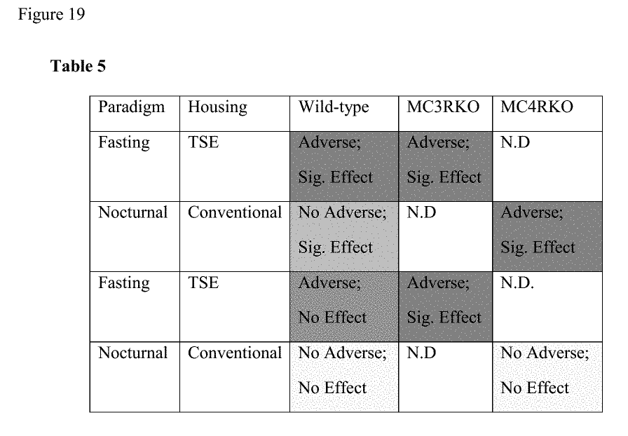

Of note some adverse reactions were observed during the TSE cage experiments that were not observed during the conventional cage experiments (FIG. 19, Table 5). These adverse reactions began with the individual mouse putting its tail upright in the air then increased sporadic activity about 15-30 minutes post-compound administration. Then the mouse went through 10-15 second bouts of “barrel roll” type behavior. This behavior was repeated 2-3 times with approximately 4-5 minutes between bouts. At which point the mouse either died, or completely recovered. All mice were completely recovered within two hours (unless there was death).

During the fasting paradigm, four adverse reactions were observed within 30 minutes of injection, and one mouse died about 2 hours post-injection. During the nocturnal paradigm there was a total of 2 adverse reactions and one mouse died 30 minutes post-administration. Mice experiencing adverse reactions recovered rapidly (<1 hr). Due to the lack of significant effects observed in the nocturnal paradigm group as well as in the ambulatory activity measurements in both paradigms (FIGS. 10A-10D and FIGS. 12A-12D), the adverse reactions observed do not seem to be having a significant effect on the parameters measured during the experiments. If the effects of the compound were due to toxicity, it would be expected that the mice receiving compound in the nocturnal paradigm would be adversely effected as well, and decreases in food intake, RER, energy expenditure, and water intake would be observed after compound administration. Furthermore, visceral illness and toxicity is usually accompanied by decreased activity, however no significant differences were observed in activity between saline and CJL-5-58 in the fasting paradigm in the TSE cages. In the nocturnal paradigm, an increase in activity was observed during the first 5 hours after administration which is inconsistent with toxic effects associated with a compound.

### EXAMPLE 7

### Effects of Co-Administration of CJL-1-14 and CJL-1-80 In Vivo

In order to help elucidate if the in vivo effects were due to the MUmBL design or were due to the co-treatment of an agonist and antagonist, co-administration experiments were performed in the same mice as the CJL-5-58 experiment with 5 nmols of CJL-1-14, Ac-His-DPhe-Arg-Trp-NH₂, and 5 nmols of CJL-1-80, Ac-His-DNal(2′)-Arg-Trp-NH₂, that would reconstitute the 10 nmols of tetrapeptide scaffolds administered with CJL-5-58 at the 5 nmol dose. In the fasting paradigm and the nocturnal paradigm, no statistically significant effect on food intake or water intake were observed compared to saline within 24 hours of administration expect for in the 2 hours timepoint in the nocturnal paradigm (FIG. 9A and 9C, FIG. 11A and 11C). There was a significant decrease in food intake 2 hours post-administration compared to saline in the nocturnal paradigm, but no other time points were significant. These data are consistent with the hypothesis in the field that co-administration of an antagonist with an agonist cancels out the effects on food intake (Fan, W. et al. Nature 1997, 385, 165-168. No significant effect was observed in energy expenditure between saline and co-administration of CJL-1-80 and CJL-1-14 in the fasting paradigm (FIG. 9D, FIG. 10D). In the nocturnal paradigm, there was a significant effect on energy expenditure observed 19 hours after administration, but no other time point was significant (FIG. 11D, FIGS. 12A-12D). The RER was significantly lower than saline in the fasting paradigm at the 1 hour and 4 hour time points (FIG. 9B). The RER was also lower in the nocturnal paradigm at 1-4 hour time points (FIG. 11B).

A direct comparison of CJL-5-58 to the co-administration of CJL-1-14 and CJL-1-80 reveals some differences. There was significant differences in the food intake at 2 and 4 hour time point in the fasting paradigm comparing CJL-5-58 to the tetrapeptide combination. There was significant differences in RER at the 2, 3, 6, and 7 hour time points in the fasting paradigm. Energy expenditure was also significantly higher for CJL-5-58 at the 13 and 17 hour time points in the fasting paradigm. In the nocturnal paradigm, no significant differences were observed for any parameter between the co-administration of the tetrapeptide combination and CJL-5-58.

The combination of CJL-1-14 and CJL-1-80 resulted more adverse observations than CJL-5-58. In the fasting paradigm, three mice died after compound administration. In the nocturnal paradigm, one mouse died. As with CJL-5-58, in the energy homeostasis parameters measured compound toxicity was not observed. If compound toxicity was suspect for the in vivo effects on energy homeostasis, it would be expected that decreases in food intake and water intake would be observed in both the fasting paradigm and the nocturnal paradigm. Furthermore, ambulatory activity resulted in very little significant changes compared to saline. The only significant changes observed in ambulatory activity were at hours 18 and 19 post-administration. It would be expected in the case of compound toxicity, a more significant effect on activity would be observed.

### EXAMPLE 8

### CJL-5-58 Administration into Melanocortin Knockout Mice

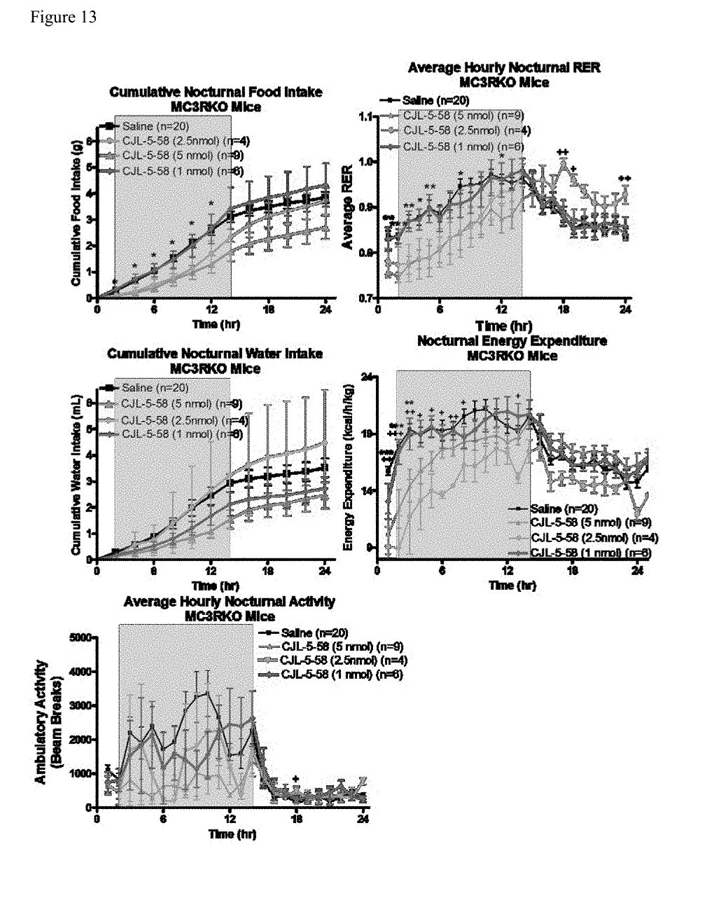

In order to more clearly understand the effects of the biased agonism at the MC4R, the effects of administration of CJL-5-58 was explored in MC3R knockout (KO) mice and MC4RKO mice. In male MC3RKO mice, a significant decrease in food intake was observed in the nocturnal feeding paradigm (FIG. 13). Food intake was decreased at time points 2-12 hours after compound ICV administration at the 5 nmol. Furthermore, RER was significantly decreased at time points 1-5, 8 and 12 hours after compound administration. Also significant increased RER was observed 18, 19, and 24 hours after 2.5 nmol compound administration. Finally, energy expenditure was significantly reduced at time points 1-3 hrs after 5 nmol administration compared to saline. After 2.5 nmol CJL-5-58 administration, energy expenditure was significant reduced 1-8 and 13 hours post-treatment. It must be taken into account that adverse reactions were also observed at the 2.5 and 5 nmol concentrations which may be a confounding factor in interpretation of this data (FIG. 19, Table 5). Adverse reactions included two mice dying after the 5 nmol dose. It should also be noted that although no significant reductions in activity were observed, the activity for CJL-5-58 at 5 nmols trended towards being lowered (p=0.069) and the error with the activity at 2.5 nmols is high. The large error in activity may serve as an indication of toxicity. It is currently difficult to decipher whether the adverse reactions observed are due to paradigm effects, genotype difference, or compound toxicity. No adverse reactions were observed at the 1 nmol dose in the nocturnal paradigm, however, no significant effects were observed at the 1 nmol dose.

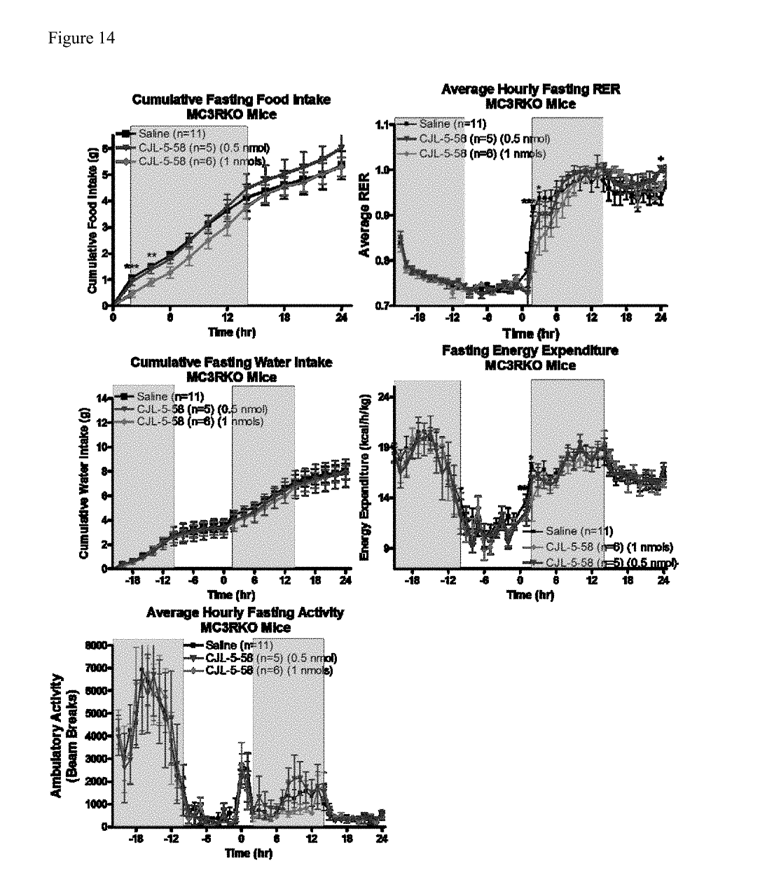

The effects of CJL-5-58 on male MC3RKO mice in a fasting-refeeding paradigm were evaluated for comparison with wild type mice. However due to the adverse reactions observed with higher dosing in the nocturnal paradigm, the effects were only evaluated at 0.5 and 1 nmols (FIG. 14). Significant reduction in food intake was observed at 2 and 4 hour time points after the 1.0 nmol CJL-5-58. Significant reductions in energy expenditure were observed in the fasting paradigm at time points 1 and 2 hours at the 1 nmol dose. RER was significantly reduced at 2 and 3 hours after the 1 nmol administration of CJL-5-58. The only significant effect observed at 0.5 nmol dosing was a significant increase in RER at time point 24 hours. No other parameters were significantly affected by administration of 0.5 nmols of CJL-5-58. It should be noted that some minor signs of adverse reactions (such as tail going upright without barrel rolls) were observed with administration of CJL-5-58 at 1 nmol in the fasting paradigm that could confound these results (FIG. 19, Table 5).

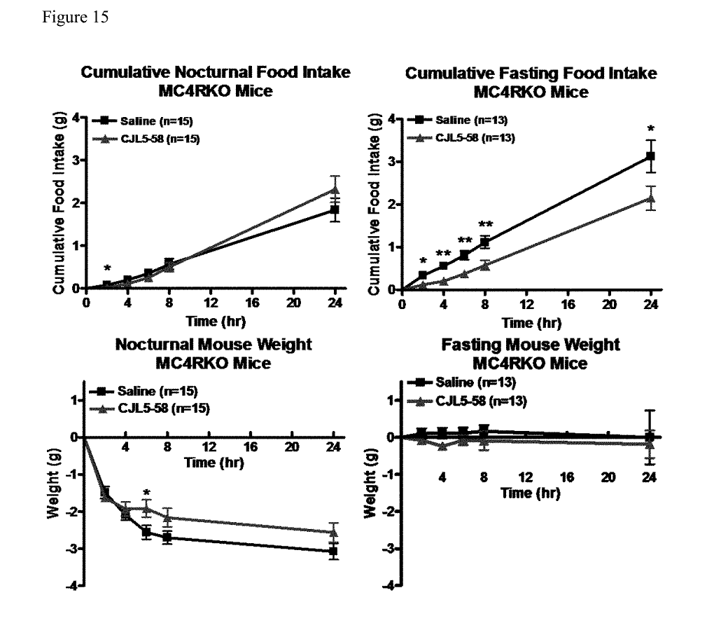

In male MC4RKO mice, CJL-5-58 was administered at 5 nmols in both a nocturnal paradigm and a fasting-refeeding paradigm in standard conventional cages (FIG. 15). The dose was well handled in the nocturnal paradigm, but minimal effect was observed. There was an immediate reduction in food intake 2 hours after compound administration. There was also a significant increase in body weight 6 hours after compound administration. In the fasting-refeeding paradigm, administration of CJL-5-58 at 5 nmols resulted in decreased food intake at 2, 4, 6, 8 and 24 hours after compound administration with no effect on mouse body weight. Mice looked healthy in the nocturnal paradigm, however some signs of adverse reactions (such as tail going upright without barrel rolls) were observed in the fasting-refeeding paradigm (FIG. 19, Table 5).

Due to the observed adverse physiological effects that were observed in the different housing and different genotypes (FIG. 19, Table 5), the exact in vivo pharmacology for MUmBL CJL-5-58 will need further elucidation. However, there are some key conclusions that may be drawn. First the adverse reactions seem to be acute and short-term (<1 h), suggesting that longer effects are due to the ligands on-target pharmacology. Second, the observed adverse reactions were increased during the fasting-refeeding paradigm, suggesting the adverse reactions are paradigm related and probably has a very specific pharmacological cause that remains to be identified. Third, the adverse behaviors appeared to be more notable in the MC3RKO mice, followed by the wild type mice, and minimal in the MC4RKO mice. This may suggest the melanocortin pathway may play a role, but experimental evidence would be necessary.

### EXAMPLE 9

### Administration of CJL-1-124 to WT, MC3RKO, and MC4RKO Mice in Metabolic Cages

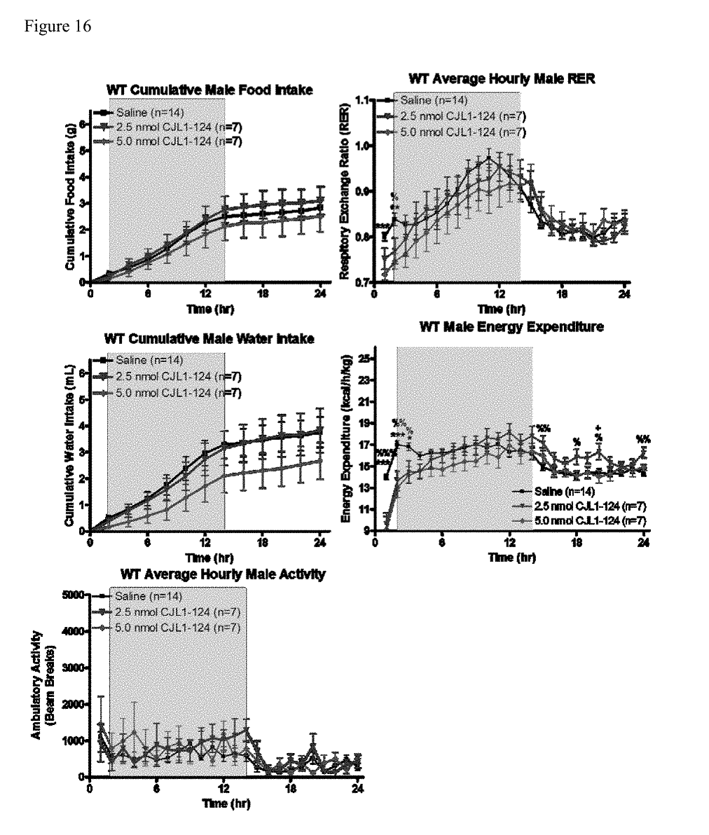

In order to study the effects of the orientation of the tetrapeptide scaffolds at the N-terminus and the C-terminus in vivo, CJL-1-124 was administered to wild type, MC3RKO, and MC4RKO mice and parameters about their energy homeostasis was recorded using TSE phenotypic metabolic cages. In preliminary conventional cages experiments, strong adverse effects as described above were observed after compound administration in the fasting paradigm, therefore only the nocturnal paradigm was performed. No significant effect on male wild type mice was observed in the nocturnal paradigm at either the 2.5 nmol or 5 nmol dose of CJL-1-124 compared to saline on food intake, water intake, or activity (FIG. 16). Compound CJL-1-124 did appear to cause a dose dependent decrease in RER at the first 2 hours after compound administration. Energy expenditure appeared to be lowered by both the 2.5 nmol and the 5 nmol dose of CJL-1-124 for the first 3 hours after administration compared to saline. Furthermore, it was observed that the 2.5 nmol dose of CJL-1-124 significantly increased the energy expenditure 15, 18, 20, and 24 hours post compound administration compared to saline. There were some signs of adverse reactions observed at higher 5 nmol dose in the wild type nocturnal paradigm.

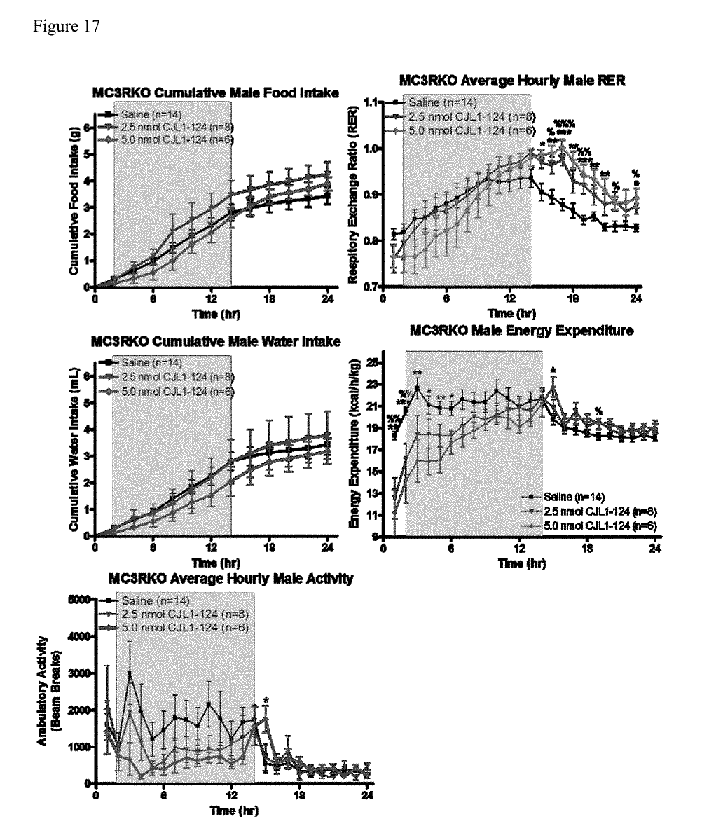

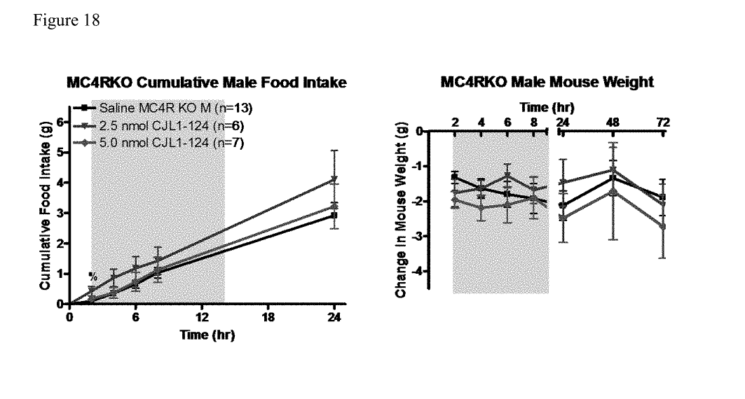

The administration of 2.5 nmols or 5.0 nmols CJL-1-124 to male MC3RKO mice resulted in no significant changes in food intake or water intake (FIG. 17). Energy expenditure was significantly reduced 1-6 hours after administration of 5.0 nmols of CJL-1-124. A significant increase in energy expenditure was observed 15 hours after compound administration. A significant reduction in energy expenditure was observed 1 and 2 hours after administration of 2.5 nmols of CJL-1-124, and a significant increase at 19 hours after compound administration. The RER was significantly increased from 15-21 and 24 hours after administration of 5 nmols of CJL-1-124. The RER of was significantly increased from 16-17, 19, 22, and 24 hours after administration of 2.5 nmols of CJL-1-124. The activity was significantly increased at 15 hours after 5 nmols of CJL-1-124, otherwise no significant changes were observed in the male MC3RKO mice. The administration of CJL-1-124 to MC4RKO mice resulted in minimal significant change in food intake or body weight. The only significant change was the increase in food intake observed 2 hours after administration of 2.5 nmol CJL-1-124 (FIG. 18). It should again be noted that some signs of toxicity were observed in the MC3RKO and the MC4RKO mice including animal death after administration.

All publications, patents, and patent documents (including Lensing, C. J. et al. J Med. Chem. 2018, Just Accepted, DOI: 10.1021/acs.jmedchem.8b00238) are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula I:
Y—X—Z I
or a salt thereof, wherein:
X is a linking group; and
Y is a melanocortin receptor agonist and Z is a melanocortin receptor antagonist; or Y is a melanocortin receptor antagonist and Z is a melanocortin agonist.

2. The compound of claim 1, wherein the melanocortin receptor agonist comprises an amino acid sequence of His-DPhe-Arg-Trp (SEQ ID NO:1).

3. The compound of claim 1, wherein the melanocortin receptor antagonist comprises an amino acid sequence of His-DNal(2′)-Arg-Trp (SEQ ID NO:2).

4. The compound of claim 1 which has the following formula II:
CH₃C(═O)-A-X—B—NH₂ II
or a salt thereof, wherein:
A is -His-DPhe-Arg-Trp-(SEQ ID NO: 1), -His-DNal(2′)-Arg-Trp-(SEQ ID NO: 2), -DTrp-DArg-Phe-DHis-, or
-DTrp-DArg-Nal(2′)-DHis-;
B is -His-DPhe-Arg-Trp-(SEQ ID NO: 1), -His-DNal(2′)-Arg-Trp-(SEQ ID NO: 2), -DTrp-DArg-Phe-DHis-, or
-DTrp-DArg-Nal(2′)-DHis-;
X is a linking group;
His is a residue of L-histidine;
DHis is a residue of D-histidine;
Phe is a residue of L-phenylalanine, wherein the phenyl ring is optionally substituted with one or more groups selected from halo, (C₁-C₄)alkyl, —O(C₁-C₄)alkyl, (C₁-C₄)haloalkyl, or —O(C₁-C₄)haloalkyl;
DPhe is a residue of D-phenylalanine, wherein the phenyl ring is optionally substituted with one or more groups selected from halo, (C₁-C₄)alkyl, —O(C₁-C₄)alkyl, (C₁-C₄)haloalkyl, or —O(C₁-C₄)haloalkyl;
Arg is a residue of L-arginine;
DArg is a residue of D-arginine;
Trp is a residue of L-tryptophan, wherein the indolyl ring is optionally substituted with one or more groups selected from halo, (C₁-C₄)alkyl, —O(C₁-C₄)alkyl, (C₁-C₄)haloalkyl, or —O(C₁-C₄)haloalkyl;
DTrp is a residue of D-tryptophan, wherein the indolyl ring is optionally substituted with one or more groups selected from halo, (C₁-C₄)alkyl, —O(C₁-C₄)alkyl, (C₁-C₄)haloalkyl, or —O(C₁-C₄)haloalkyl;
Nal(2′) is a residue of L-2-naphthyl-alanine, wherein the phenyl ring is optionally substituted with one or more groups selected from halo, (C₁-C₄)alkyl, —O(C₁-C₄)alkyl, (C₁-C₄)haloalkyl, or —O(C₁-C₄)haloalkyl; and
DNal(2′) is a residue of D-2-naphthyl-alanine, wherein the phenyl ring is optionally substituted with one or more groups selected from halo, (C₁-C₄)alkyl, —O(C₁-C₄)alkyl, (C₁-C₄)haloalkyl, or —O(C₁-C₄)haloalkyl;
provided if A is -His-DPhe-Arg-Trp-(SEQ ID NO: 1), B is not -His-DPhe-Arg-Trp-(SEQ ID NO: 1), wherein the phenyl ring and the indolyl ring are not substituted;
and provided if A is -His-DNal(2′)-Arg-Trp-(SEQ ID NO: 2), B is not -His-DNal(2′)-Arg-Trp-(SEQ ID NO: 2), wherein the naphthyl ring and the indolyl ring are not substituted.

5. The compound of claim 4, wherein A is -His-DPhe-Arg-Trp-(SEQ ID NO: 1) or -His-DNal(2′)-Arg-Trp-(SEQ ID NO: 2).

6. The compound of claim 4. wherein A is:

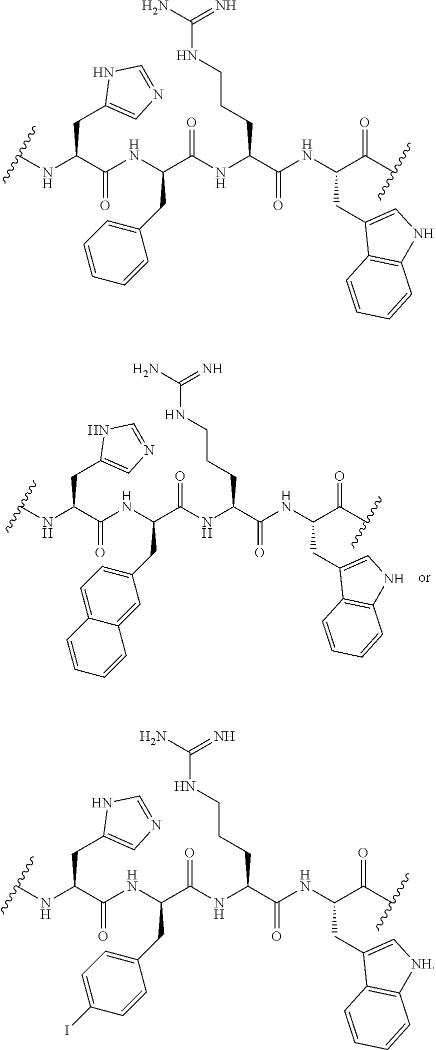 [SMILES: *N[C@@H](Cc1cnc[nH]1)C(=O)N[C@H](Cc1ccccc1)C(=O)N[C@@H](CCCNC(=N)N)C(=O)N[C@@H](Cc1c[nH]c2ccccc12)C(*)=O ; *N[C@@H](Cc1cnc[nH]1)C(=O)N[C@H](Cc1ccc2ccccc2c1)C(=O)N[C@@H](CCCNC(=N)N)C(=O)N[C@@H](Cc1c[nH]c2ccccc12)C(*)=O ; "or" ; *N[C@@H](Cc1cnc[nH]1)C(=O)N[C@H](Cc1ccc(I)cc1)C(=O)N[C@@H](CCCNC(=N)N)C(=O)N[C@@H](Cc1c[nH]c2ccccc12)C(*)=O]

7. The compound of claim 4, wherein B is -His-DPhe-Arg-Trp-(SEQ ID NO: 1) or -His-DNal(2′)-Arg-Trp-(SEQ ID NO: 2).

8. The compound of claim 4, wherein B is:

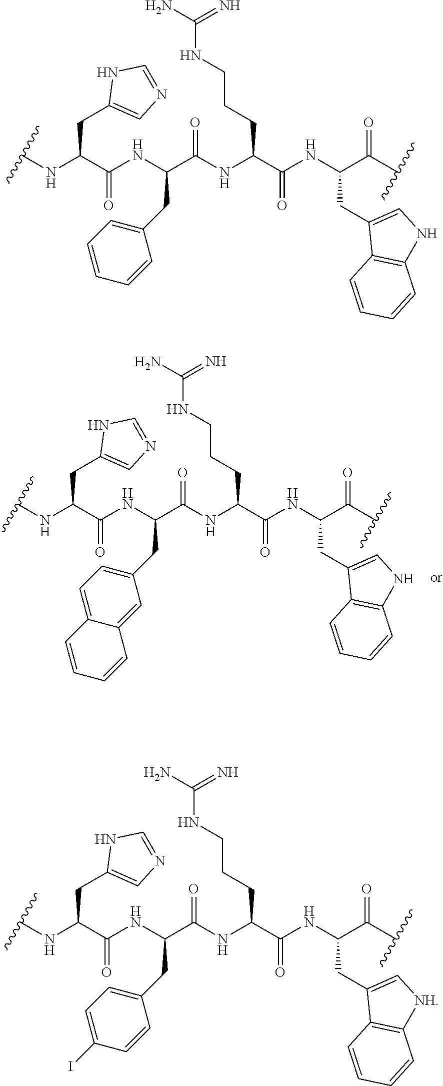 [SMILES: *N[C@@H](Cc1cnc[nH]1)C(=O)N[C@H](Cc1ccccc1)C(=O)N[C@@H](CCCNC(=N)N)C(=O)N[C@@H](Cc1c[nH]c2ccccc12)C(*)=O ; *N[C@@H](Cc1cnc[nH]1)C(=O)N[C@H](Cc1ccc2ccccc2c1)C(=O)N[C@@H](CCCNC(=N)N)C(=O)N[C@@H](Cc1c[nH]c2ccccc12)C(*)=O ; "or" ; *N[C@@H](Cc1cnc[nH]1)C(=O)N[C@H](Cc1ccc(I)cc1)C(=O)N[C@@H](CCCNC(=N)N)C(=O)N[C@@H](Cc1c[nH]c2ccccc12)C(*)=O]

9. The compound of claim 1 which is a compound of formula IIa: 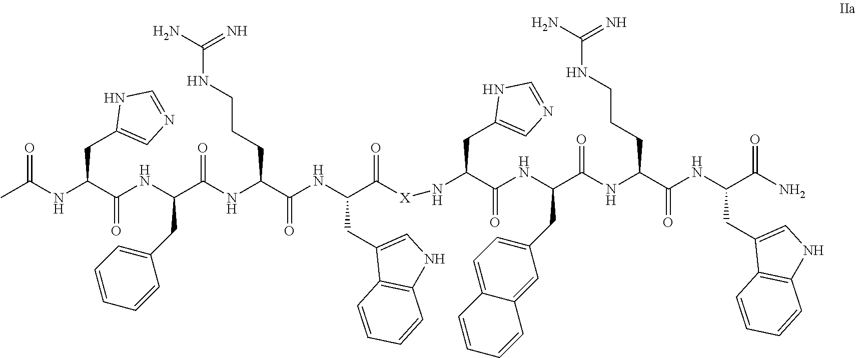
or a salt thereof.

10. The compound of claim 1 which is a compound of formula IIb: 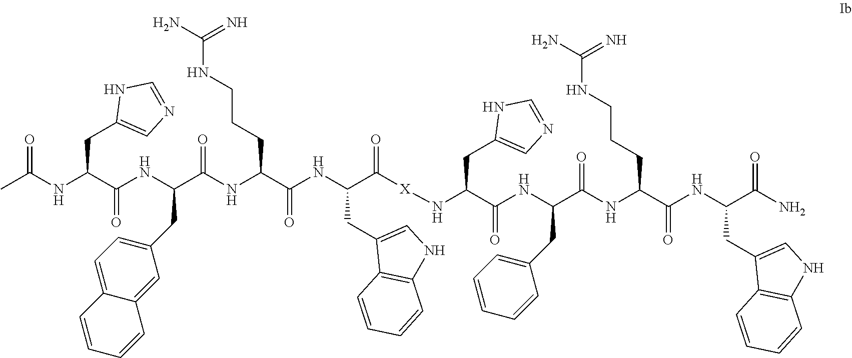
or a salt thereof.

11. The compound of claim 10, wherein X is —NH(CH₂CH₂O)ₙCH₂CH₂C(O)—, wherein n is 1, 2, 3, 4, 5, 6, 7, 8, 9, or 10.

12. The compound of claim 1, wherein X is:

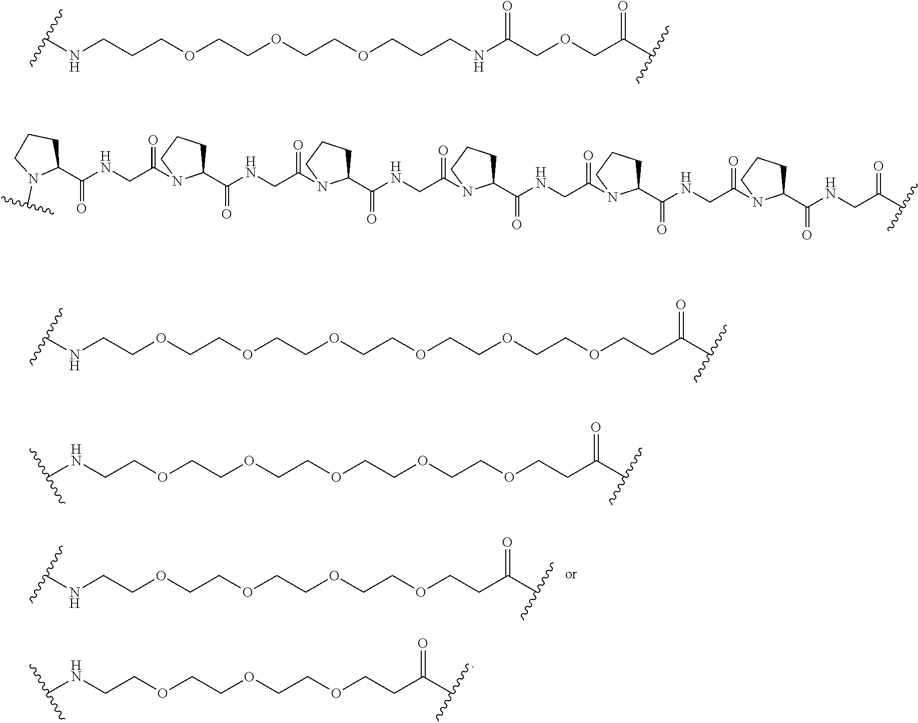

13. The compound of claim 1, which is selected from the group consisting of:
Ac-His-DPhe(p-I)-Arg-Trp-(PEDG20)-His-DPhe(p-I)-Arg-Trp-NH₂;
Ac-His-DNal(2)-Arg-Trp-(Pro-Gly)₆-His-DPhe-Arg-Trp-NH₂;
Ac-His-DPhe-Arg-Trp-(PEDG20)-His-DNal(2′)-Arg-Trp-NH₂;
Ac-His-DNal(2′)-Arg-Trp-(PEDG20)-His-DPhe-Arg-Trp-NH₂;
Ac-His-DNal(2′)-Arg-Trp-(PEDG20)-(PEDG20)-His-DPhe-Arg-Trp-NH₂;
Ac-His-DPhe(p-I)-Arg-Trp-(Pro-Gly)₆-His-DPhe-Arg-Trp-NH₂;
Ac-His-DPhe(p-I)-Arg-Trp-(PEDG20)-His-DPhe-Arg-Trp-NH₂;
Ac-His-DPhe-Arg-Trp-(PEDG20)-His-DPhe(p-I)-Arg-Trp-NH₂;
Ac-His-DNal(2′)-Arg-Trp-(PEDG20)-His-DPhe(p-I)-Arg-Trp-NH₂;
Ac-His-DPhe(p-I)-Arg-Trp-(PEDG20)-His-DNal(2′)-Arg-Trp-NH₂;
Ac-His-DPhe-Arg-Trp-(PEG)2(22atoms)-His-DNal(2′)-Arg-Trp-NH₂;
Ac-His-DPhe-Arg-Trp-(PEDG20)-His-DNal(2′)-Arg-Trp-NH₂;
Ac-His-DPhe-Arg-Trp-(PEG)(19atoms)-His-DNal(2′)-Arg-Trp-NH₂;
Ac-DTrp-DArg-Phe-DHis-(PEG)(22 atoms)-His-DPhe-Arg-Trp-NH₂;
Ac-DTrp-DArg-Phe-DHis-(PEDG20)-His-DPhe-Arg-Trp-NH₂;
Ac-DTrp-DArg-Phe-DHis-(PEG)(19 atoms)-His-DPhe-Arg-Trp-NH₂;
Ac-His-DPhe-Arg-Trp-(PEG)(22 atoms)-DTrp-DArg-Phe-DHis-NH₂;
Ac-His-DPhe-Arg-Trp-(PEDG20)-DTrp-DArg-Phe-DHis-NH₂;
Ac-His-DPhe-Arg-Trp-(PEG)(19 atoms)-DTrp-DArg-Phe-DHis-NH₂;
Ac-DTrp-DArg-Phe-DHis-(PEG)(22 atoms)-DTrp-DArg-Phe-DHis-NH₂;
Ac-DTrp-DArg-Phe-DHis-(PEDG20)-DTrp-DArg-Phe-DHis-NH₂; and
Ac-DTrp-DArg-Phe-DHis-(PEG)(19 atoms)-DTrp-DArg-Phe-DHis-NH₂;
and salts thereof, wherein:
Ac is CH₃C(═O)—;
His is a residue of L-histidine;
DHis is a residue of D-histidine;
Phe is a residue of L-phenylalanine;
DPhe is a residue of D-phenylalanine;
Arg is a residue of L-arginine;
DArg is a residue of D-arginine;
Trp is a residue of L-tryptophan;
DTrp is a residue of D-tryptophan;
DNal(2′) is a residue of D-2-naphthyl-alanine;
DPhe(p-I) is a residue of D-para-iodo-phenylalanine;

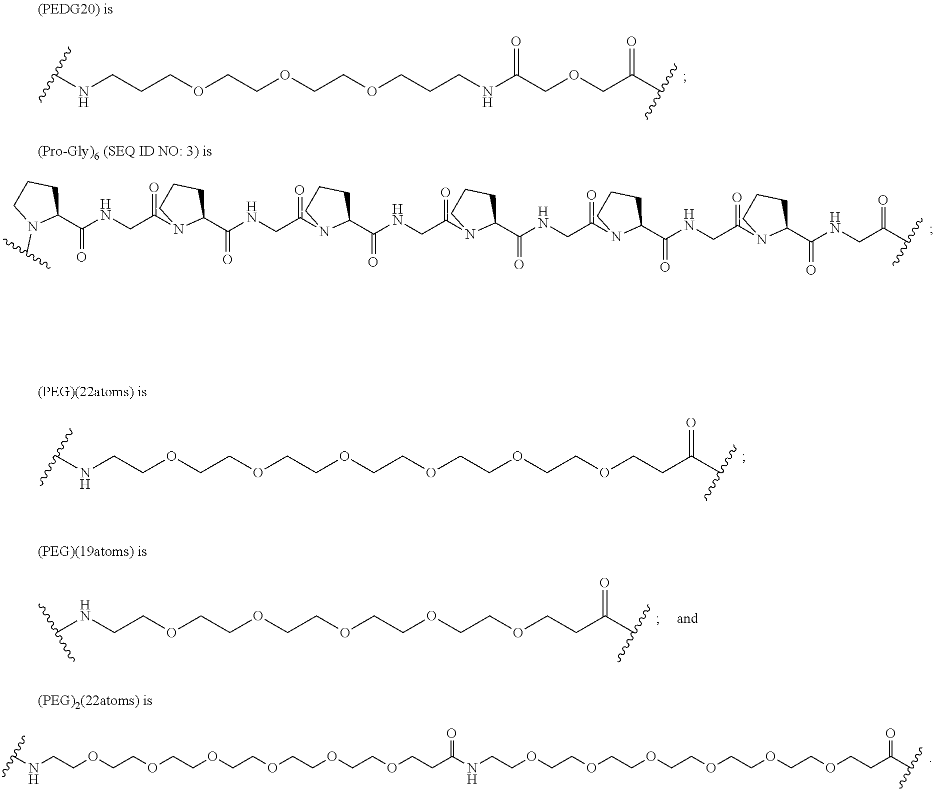 [SMILES: *NCCCOCCOCCOCCCNC(=O)COCC(*)=O ; *C(=O)CNC(=O)[C@@H]1CCCN1C(=O)CNC(=O)[C@@H]1CCCN1C(=O)CNC(=O)[C@@H]1CCCN1C(=O)CNC(=O)[C@@H]1CCCN1C(=O)CNC(=O)[C@@H]1CCCN1C(=O)CNC(=O)[C@@H]1CCCN1* ; *NCCOCCOCCOCCOCCOCCOCCC(*)=O ; *NCCOCCOCCOCCOCCOCCC(*)=O ; *NCCOCCOCCOCCOCCOCCOCCC(=O)NCCOCCOCCOCCOCCOCCOCCC(*)=O]

14. The compound of claim 1, which is
Ac-His-DNal(2′)-Arg-Trp-(Pro-Gly)₆-His-DPhe-Arg-Trp-NH₂;
Ac-His-DPhe-Arg-Trp-(PEDG20)-His-DNal(2′)-Arg-Trp-NH₂;
Ac-His-DNal(2′)-Arg-Trp-(PEDG20)-His-DPhe-Arg-Trp-NH₂; or
Ac-His-DNal(2′)-Arg-Trp-(PEDG20)-(PEDG20)-His-DPhe-Arg-Trp-NH₂;
or a salt thereof, wherein:
Ac is CH₃C(═O)—;
His is a residue of L-histidine;
DPhe is a residue of D-phenylalanine;
Arg is a residue of L-arginine;
Trp is a residue of L-tryptophan;
DNal(2′) is a residue of D-2-naphthyl-alanine;

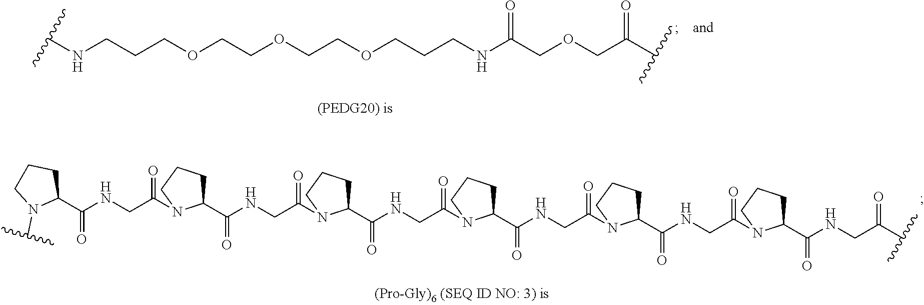 [SMILES: *NCCCOCCOCCOCCCNC(=O)COCC(*)=O ; *C(=O)CNC(=O)[C@@H]1CCCN1C(=O)CNC(=O)[C@@H]1CCCN1C(=O)CNC(=O)[C@@H]1CCCN1C(=O)CNC(=O)[C@@H]1CCCN1C(=O)CNC(=O)[C@@H]1CCCN1C(=O)CNC(=O)[C@@H]1CCCN1*]

15. The compound of claim 1 which is: 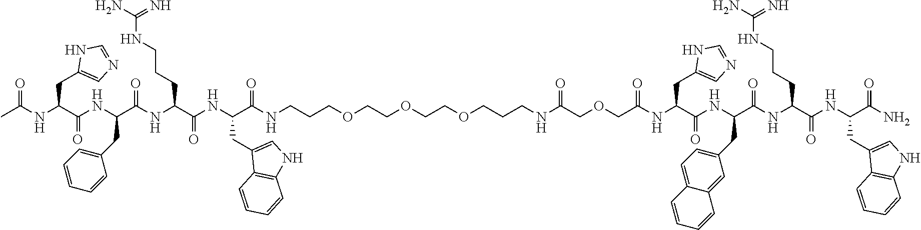 [SMILES: CC(=O)N[C@@H](Cc1cnc[nH]1)C(=O)N[C@H](Cc1ccccc1)C(=O)N[C@@H](CCCNC(=N)N)C(=O)N[C@@H](Cc1c[nH]c2ccccc12)C(=O)NCCCOCCOCCOCCCNC(=O)COCC(=O)N[C@@H](Cc1cnc[nH]1)C(=O)N[C@H](Cc1ccc2ccccc2c1)C(=O)N[C@@H](CCCNC(=N)N)C(=O)N[C@@H](Cc1c[nH]c2ccccc12)C(N)=O]
or a salt thereof.

16. A compound comprising first amino acid sequence having at least 80% sequence identity to His-DPhe-Arg-Trp (SEQ ID NO:1), further comprising second amino acid sequence at least 80% identity to His-DNal(2′)-Arg-Trp (SEQ ID NO:2), or a salt thereof.

17. A pharmaceutical composition comprising a compound as described in claim 1, or a pharmaceutically acceptable salt thereof, and a pharmaceutically acceptable carrier.

18. A dietary supplement comprising a compound as described in claim 1, or a salt thereof.

19. A method of treating obesity or a disease associated with obesity in an animal in need thereof, comprising administering an effective amount of a compound as described in claim 1, or a pharmaceutically acceptable salt thereof, to the animal.

20. A method of modulating the activity of a melanocortin receptor in vitro or in vivo comprising contacting the receptor with an effective amount of a compound as described in claim 1, or a pharmaceutically acceptable salt thereof.

21. A method of modulating the activity of a melanocortin receptor homodimer in vitro or in vivo comprising contacting the homodimer with an effective amount of a compound as described in claim 1, or a pharmaceutically acceptable salt thereof.

22. A method of activity cAMP signaling and simultaneously blocking β-arrestin recruitment in vitro or in vivo comprising contacting a melanocortin receptor homodimer with an effective amount of a compound as described in claim 1, or a pharmaceutically acceptable salt thereof.

23. A method of modulating appetite in an animal in need thereof, comprising administering an effective amount of a compound as described in claim 1, or a pharmaceutically acceptable salt thereof, to the animal.

24. A method of modulating metabolic activity in an animal in need thereof, comprising administering an effective amount of a compound as described in claim 1, or a pharmaceutically acceptable salt thereof, to the animal.

25. A method of decreasing food intake, reducing body fat percentage, and/or increasing fat consumption in an animal in need thereof, comprising administering an effective amount of compound as described in claim 1, or a pharmaceutically acceptable salt thereof, to the animal.

26. A method of activating one downstream signaling event and simultaneously blocking a different downstream signaling event of a G protein-couple receptor (GPCR) homodimer comprising contacting the GPCR homodimer a ligand that comprises an agonist pharmacophore and an antagonist pharmacophore, wherein the agonist pharmacophore occupies and activates one receptor within the GPCR homodimer and the antagonist pharmacophore occupies and deactivates the other receptor within the GPCR homodimer.
